(19) United States
(12) Patent Application Publication
(10) Pub. No.: US 2026/0200988 A1
(43) Pub. Date: Jul. 16, 2026

(54) LIGANDS SPECIFIC FOR ASCT1

(71) Applicants: METAFORA BIOSYSTEMS, Paris (FR); CENTRE NATIONAL DE LA RECHERCHE SCIENTIFIQUE, Paris (FR); UNIVERSITÉ DE MONTPELLIER, Montpellier (FR); THE UNITED STATES OF AMERICA, AS REPRESENTED BY THE SECRETARY, DEPARTMENT OF HEALTH AND HUMAN, Bethesda, MD (US)

(72) Inventors: Jacinthe MEKARY, Montpellier (FR); Jawida TOUHAMI, Montpellier (FR); Philippe YOUKHARIBACHE, Bethesda, MD (US); Donatella GIOVANNINI, Paris (FR); Marc SITBON, Montpellier (FR)

(73) Assignees: METAFORA BIOSYSTEMS, Paris (FR); CENTRE NATIONAL DE LA RECHERCHE SCIENTIFIQUE, Paris (FR); UNIVERSITÉ DE MONTPELLIER, Montpellier (FR); THE UNITED STATES OF AMERICA, AS REPRESENTED BY THE SECRETARY, DEPARTMENT OF HEALTH AND HUMAN, Bethesda, MD (US)

(21) Appl. No.: 19/135,890

(22) PCT Filed: Dec. 6, 2023
(86) PCT No.: PCT/EP2023/084599

§ 371 Date: Jun. 5, 2025

(30) Foreign Application Priority Data
Dec. 6, 2022 (EP) 22306795.0

(51) Int. Cl. C07K 14/005; A61K 38/00; G01N 33/575

(52) U.S. Cl. CPC C07K 14/005; G01N 33/5758; A61K 38/00; C12N 2740/13022; C12N 2740/13033

(57) ABSTRACT

Polypeptides capable of specifically recognizing and binding to the neutral amino acid transporter ASCT1/SLC1A4, while not binding to the related neutral amino acid transporter ASCT2/SLC1A5. Also, both in vitro and in vivo methods of specifically detecting or/and measuring the level of ASCT1, and to the use of said polypeptides in diagnosis and therapy.

### FIELD OF INVENTION

The present invention relates to polypeptides capable of specifically recognizing and binding to the neutral amino acid transporter ASCT1/SLC1A4, while not binding to the related neutral amino acid transporter ASCT2/SLC1A5. The present invention further relates to in vitro and in vivo methods of specifically detecting or/and measuring the level of ASCT1, and to the use of said polypeptides in diagnosis and therapy.

### BACKGROUND OF INVENTION

Solute Carrier (SLC) transporters form a family of more than 450 membrane-bound proteins which facilitate the transport of a wide range of substrates through biological membranes. Thus, SLC transporters have important roles in physiological processes ranging from the cellular uptake of nutrients to the absorption of drugs and other xenobiotics.

The mammalian members of the Solute Carrier 1A (SLC1A) family include the Alanine Serine Cysteine Transporters (ASCT) and the Excitatory Amino Acid Transporters (EAAT). ASCT subfamily counts two members: Solute Carrier family 1A member 4 (SLC1A4), also known as ASCT1, and Solute Carrier family 1A member 5 (SLC1A5), also known as ASCT2. Both ASCT1 and ASCT2 are sodium-dependent transporters. ASCT1 is largely selective for cysteine, alanine, and serine, but can also transport threonine, asparagine, and to a lesser extent, proline, while ASCT2 is selective for alanine, serine, cysteine, threonine, glutamine and asparagine and can also transport to a lesser extend methionine, glycine, leucine, valine and glutamic acid.

The three-dimensional structure of both ASCT1 and ASCT2 revealed a homotrimeric assembly with a one-gate elevator mechanism, eight transmembrane helices and two helical hairpins. In addition, human ASCT1 shares 57% amino acid sequence identity with human ASCT2 and an almost overlapping three-dimensional architecture.

In addition to the traditional physiological and pathological function of SLCs in the human body, some SLCs act as cell surface receptors for viruses. In particular, the spleen necrosis virus (SNV) is able to bind both ASCT1/SLC1A4 and ASCT2/SLC1A5 and use them as viral entry receptors.

ASCT1 has been shown to be broadly distributed in the human body. Indeed, high levels of ASCT1 mRNA transcripts are found in the brain, skeletal muscle, lung, kidney, ovaries, heart, pancreas and across the digestive tract.

The physiological role of ASCT1 has been investigated since its first identification. However, limited information is available on this matter, most likely because of the lack of reagents to detect it at the cell surface. ASCT1 is the main transporter for serine in the central nervous system and the most acknowledged function of ASCT1 seems to be linked with brain homeostasis due to the transport of D-serine, which is considered a gliotransmitter.

Studies of the central nervous system have shown that children with loss-of-function mutations in the SLC1A4 gene manifest neurological pathology. Additionally, ASCT1 mutations are linked to alterations in brain development and function such as schizophrenia, visual dysfunction, amyotrophic lateral sclerosis (ALS), microcephaly in children disorder, and the inherited disease SPATCCM (spasticity and thin corpus callosum and progressive microcephaly).

Furthermore, as intracellular levels of serine are maintained through the uptake of exogenous pools by serine transporters, it was demonstrated that ASCT1 is associated with cancer processes, providing anabolic precursors of the building blocks that support cancer cell proliferation. Thus far, ASCT1 has been associated with esophageal adenocarcinomas, prostate cancer, pancreatic ductal adenocarcinoma, hepatocellular carcinoma, micropapillary carcinoma, and refractory diffuse large B-cell lymphoma (DLBCL). ASCT1 has also been identified as upregulated in neuroblastoma tumor-initiating cells, and may constitute a therapeutic target for the treatment of this cancer. Additionally, it was shown that an upregulation of ASCT1 transporter occurs in liposarcoma.

Therefore, ASCT1 represents an interesting target for the treatment of pathologies such as cancers and diseases and disorders of the central nervous system. However, currently available compounds capable of targeting and inhibiting ASCT1 are limited. Moreover, because of the high similarities between ASCT1 and ASCT2, most available compounds targeting ASCT1 also target ASCT2. However, ASCT1 and ACST2 are involved in the transport of distinct amino acids, and they display different functions. And, above all, they are associated to distinct diseases and disorders. Therefore, there is a need to identify compounds capable of selectively recognizing, binding and/or inhibiting ASCT1 only, while not binding to ASCT2.

The Inventors have managed to engineer some polypeptides that are specifically able to bind ASCT1, while not binding to ASCT2. Furthermore, the Inventors have demonstrated that, in addition to binding ASCT1, these polypeptides were able to specifically inhibit ASCT1 serine-transport function. Thus, such polypeptides specifically binding and inhibiting ASCT1 represent attractive tools for specifically detecting or/and measuring the level of ASCT1 in a biological sample, as well as valuable diagnostic and therapeutic tools for treating ASCT1-associated diseases and disorders.

### SUMMARY

The invention relates to a polypeptide of sequence SEQ ID NO: 1 or a functional variant thereof, which specifically binds to ASCT1 and does not specifically bind to ASCT2, wherein:

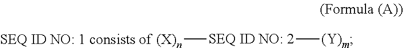

(X)ₙ is a sequence of n amino acids;
(Y)ₘ is a sequence of m amino acids;
“—” designates a peptide bound;
n≥0 and m≥0; and
if m≥1 then (Y)ₘ does not comprise or consist of the m first amino acids of the sequence SEQ ID NO: 3 or a functional variant thereof, said functional variant of the m first amino acids of the sequence SEQ ID NO: 3 being such that SEQ ID NO: 2-(Y)ₘ specifically binds to ASCT2; and
wherein the functional variant of SEQ ID NO: 1 has a sequence comprising a functional variant of SEQ ID NO: 2 instead of SEQ ID NO: 2, wherein said functional variant of SEQ ID NO: 2 specifically binds to ASCT1.

In some embodiments, the length of said polypeptide or a functional variant thereof is of less than 100 amino acids, less than 90 amino acids, less than 80 amino acids, less than 70 amino, or less than 64 amino acids.

In some embodiments, said polypeptide or functional variant has a sequence comprising SEQ ID NO: 4, SEQ ID NO: 5, SEQ ID NO: 6 or SEQ ID NO: 7.

In some embodiments, m=0.

In some embodiments, n=0 and m=0.

In some embodiments, said functional variant of the sequence SEQ ID NO: 1 has a sequence at least 80%, 85%, 90%, 95%, or 99% identical to the sequence SEQ ID NO: 1.

In some embodiments, said polypeptide or functional variant has a sequence consisting of SEQ ID NO: 2.

In some embodiments, said polypeptide or functional variant thereof is labeled with a detectable label or is coupled with a contrast agent.

The invention also relates to a nucleic acid encoding the polypeptide or functional variant thereof according to the invention, or an expression vector comprising said nucleic acid, or a cell comprising said nucleic acid or said expression vector.

The invention further relates to a diagnostic or pharmaceutical composition comprising the polypeptide or functional variant thereof according to the invention, or the nucleic acid, the expression vector or the cell according to the invention, and at least one pharmaceutically acceptable excipient.

The invention also pertains to an in vitro method of specifically detecting or/and measuring the level of ASCT1 in a sample, while not detecting or/and measuring the level of ASCT2, wherein said method comprises the steps of:
a) contacting said sample with the polypeptide or functional variant thereof according to the invention, and
b) detecting and/or measuring the binding of said polypeptide or functional variant thereof to ASCT1.

The invention also pertains to the polypeptide or functional variant thereof according to the invention, for use for specifically detecting or/and measuring the level of ASCT1 in vivo, while not detecting or/and measuring the level of ASCT2.

The invention also relates to the in vitro method according to the invention, or the use according to the invention, for diagnosing or monitoring an ASCT1-associated disease in a subject.

The invention further relates to the polypeptide or functional variant thereof according to the invention, the nucleic acid, the expression vector or the cell according to the invention, or the pharmaceutical composition according to the invention, for use as a medicament.

The invention finally relates to the polypeptide or functional variant thereof according to the invention, the nucleic acid, the expression vector or the cell according to the invention, or the pharmaceutical composition according to the invention, for use in the treatment of an ASCT1-associated disease, by specifically targeting ASCT1 and not ASCT2.

### Definitions

In the present invention, the following terms have the following meanings:

The term “about” preceding a figure means plus or less 10% of the value of said figure. It is to be understood that the figure to which the term “about” refers is itself also specifically, and preferably, disclosed.

The term “amino acid” as used herein, refers to both natural and synthetic amino acids, and both D and L amino acids. “Standard amino acid” or “naturally occurring amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. For example, naphtlylalanine can be substituted for tryptophan to facilitate synthesis. Other synthetic amino acids that can be substituted include, but are not limited to, L-hydroxypropyl, L-3,4-dihydroxyphenylalanyl, alpha-amino acids such as L-alpha-hydroxylysyl and D-alpha-methylalanyl, L-alpha-methylalanyl, beta-amino acids, and isoquinolyl. The term “amino acid” also encompasses chemically modified amino acids, including, but not limited to, salts, amino acid derivatives (such as amides), and substitutions.

The term “cancer”, as used herein, refers to any member of a class of diseases or disorders characterized by uncontrolled division of cells and the ability of these cells to invade other tissues, either by direct growth into adjacent tissue through invasion or by implantation into distant sites by metastasis. Metastasis is defined as the stage in which cancer cells are transported through the bloodstream or lymphatic system.

The term “diagnosis” as used herein, refers to medical diagnosis, the process of determining which disease explain the symptoms of a subject.

The term “diagnostic composition” refers to a composition that may be used to perform a diagnosis. It may be a composition to be used in vitro in order to perform an in vitro diagnosis, or a composition to be administered to a subject in order to perform an in vivo diagnosis.

The term “identity”, when used in a relationship between the sequences of two or more polypeptides or of two or more DNA sequences, refers to the degree of sequence relatedness between polypeptides or DNA sequences (respectively), as determined by the number of matches between strings of two or more amino acid residues or of two or more nucleotides, respectively. “Identity” measures the percent of identical matches between the smaller of two or more sequences with gap alignments (if any) addressed by a particular mathematical model or computer program (i.e., “algorithms”). Identity of related polypeptides or DNA sequences can be readily calculated by known methods. Such methods include, but are not limited to, those described in Arthur M. Lesk, Computational Molecular Biology: Sources and Methods for Sequence Analysis (New-York: Oxford University Press, 1988); Douglas W. Smith, Biocomputing: Informatics and Genome Projects (New-York: Academic Press, 1993); Hugh G. Griffin and Annette M. Griffin, Computer Analysis of Sequence Data, Part 1 (New Jersey: Humana Press, 1994); Gunnar von Heinje, Sequence Analysis in Molecular Biology: Treasure Trove or Trivial Pursuit (Academic Press, 1987); Michael Gribskov and John Devereux, Sequence Analysis Primer (New York: M. Stockton Press, 1991); and Carillo et al., 1988. SIAM J. Appl. Math. 48(5): 1073-1082. Preferred methods for determining identity are designed to give the largest match between the sequences tested. Methods of determining identity are described in publicly available computer programs. Preferred computer program methods for determining identity between two sequences include the GCG program package, including GAP (Devereux et al., 1984. Nucl. Acid. Res. 12 (1 Pt 1): 387-395; Genetics Computer Group, University of Wisconsin Biotechnology Center, Madison, WI), BLASTP, BLASTN, TBLASTN and FASTA (Altschul et al., 1990. J. Mol. Biol. 215(3): 403-410). The BLASTX program is publicly available from the National Center for Biotechnology Information (NCBI) and other sources (BLAST Manual, Altschul et al. NCB/NLM/NIH Bethesda, Md. 20894; Altschul et al., 1990. J. Mol. Biol. 215(3): 403-410). The well-known Smith Waterman algorithm may also be used to determine identity.

The term “ligand” as used herein, refers to a small molecule (including but not limited to proteins, peptides, peptidomimetic compounds and other small molecule compounds) that binds specifically to another molecule.

The term “polypeptide” refers to a linear polymer of amino acids (preferably at least 50 amino acids) linked together by peptide bonds.

The term “protein” specifically refers to a functional entity formed of one or more polypeptides, and optionally of non-polypeptides cofactors.

The term “sample”, as used herein, refers to any biological material obtained via suitable methods known to the person skilled in the art from a subject. The sample may be collected in a clinically acceptable manner, e.g., in a way that cells, nucleic acids (such as DNA and RNA), proteins and/or metabolites are preserved. A “sample” may include body tissue and/or bodily fluids.

The term “therapeutically effective amount” means level or amount of agent that is aimed at, without causing significant negative or adverse side effects to the target, (1) delaying or preventing the onset of an ASCT1-associated disease; (2) slowing down or stopping the progression, aggravation, or deterioration of one or more symptoms of an ASCT1-associated disease; (3) bringing about ameliorations of the symptoms of an ASCT1-associated disease; (4) reducing the severity or incidence of an ASCT1-associated disease; or (5) curing an ASCT1-associated disease. A therapeutically effective amount may be administered prior to the onset of an ASCT1-associated disease, for a prophylactic or preventive action. Alternatively, or additionally, the therapeutically effective amount may be administered after initiation of an ASCT1-associated disease, for a therapeutic action.

The term “treatment” refers to both therapeutic treatment and prophylactic or preventive measures; wherein the object is to prevent or slow down (lessen) an ASCT1-associated disease. Those in need of treatment include those already with an ASCT1-associated disease as well as those prone to have an ASCT1-associated disease or those in whom an ASCT1-associated disease is to be prevented. A subject or mammal is successfully “treated” for a disease if, after receiving a therapeutic amount of a compound, the patient shows observable and/or measurable reduction in or absence of one or more of the following: reduction in the number of pathogenic cells; reduction in the percentage of total cells that are pathogenic; and/or relief to some extent, of one or more of the symptoms associated with the specific disease or condition; reduced morbidity and mortality, and improvement in quality of life issues. The above parameters for assessing successful treatment and improvement in the disease are readily measurable by routine procedures familiar to a physician.

The term “subject”, as used herein, refers to an animal, preferably a mammal, more preferably a human. In one embodiment, the subject is a patient, i.e., a recipient of health care services, who/which is awaiting the receipt of, or is receiving medical care or was/is/will be the object of a medical procedure, or is monitored for the development of a disease.

### DETAILED DESCRIPTION

As used herein, “ASCT1” refers to a serine and other neutral amino acid transporter excluding glutamine, belonging to the SLC series, wherein SLC stands for SoLute Linked Carriers. ASCT1 is notably used as a receptor for the envelope glycoproteins (Env) from the human endogenous retrovirus W (HERV-W), also known as syncytin-1, the RD114 feline gammaretrovirus, the baboon endogenous virus (BaEV), the reticuloendotheliosis viruses (REV), among which the spleen necrosis virus (SNV), the simian retroviruses (SRV) and the Mason-Pfizer monkey virus (MPMV).

In some embodiments, ASCT1 is human ASCT1 (accession number AAH26216.1, SEQ ID NO: 20) encoded by SEQ ID NO: 21 (accession number HUMASCT1A). In some embodiments, ASCT1 comprises or consists of an amino acid sequence presenting a sequence identity of at least 75, 80, 85, 90, 91, 92, 93, 94, 95, 96, 97, 98, 99% or more with SEQ ID NO: 20. In some embodiments, ASCT1 is encoded by a nucleotide sequence presenting a sequence identity of at least 75, 80, 85, 90, 91, 92, 93, 94, 95, 96, 97, 98, 99% or more with SEQ ID NO: 21.

As used herein, “ASCT2” refers to a glutamine and other neutral amino acid transporter, belonging to the SLC series. ASCT2 is notably used as a receptor for the envelope glycoproteins from the human endogenous retrovirus W (HERV-W), also known as syncytin-1, the RD114 feline gammaretrovirus, the baboon endogenous virus (BaEV), the reticuloendothelial viruses (REV), including the spleen necrosis virus (SNV), the simian retroviruses (SRV) and the Mason-Pfizer monkey virus (MPMV).

In some embodiments, ASCT2 is human ASCT2 (accession number Q15758.2, SEQ ID NO: 22) encoded by SEQ ID NO: 23 (accession number GQ919058). In one embodiment, ASCT2 comprises or consists of an amino acid sequence presenting a sequence identity of at least 75, 80, 85, 90, 91, 92, 93, 94, 95, 96, 97, 98, 99% or more with SEQ ID NO: 22. In some embodiments, ASCT2 is encoded by a nucleotide sequence presenting a sequence identity of at least 75, 80, 85, 90, 91, 92, 93, 94, 95, 96, 97, 98, 99% or more with SEQ ID NO: 23.

The polypeptide of the invention may be derived from a part of the soluble part of the glycoprotein of an enveloped virus, in particular the spleen necrosis virus (SNV). SNV is an avian gammaretrovirus, which belongs to the REV family. In particular, the polypeptide of the invention is derived from a part of a receptor-binding domain (RBD) derived from the soluble part of the glycoprotein of SNV.

The term “receptor-binding domain” (RBD) designates a part or fragment of the soluble part of a glycoprotein of an enveloped virus that interacts with a cell surface receptor, such as a nutrient transporter, an integral protein, a GPI-anchored protein, a polysaccharide, a hetero- or proteoglycan or any other component of the extracellular matrix, used by the virus as a viral receptor.

The expression “derived from the soluble part of the glycoprotein of an enveloped virus” means that the polypeptide is a fragment or a part of a glycoprotein contained in the envelope of a virus and can be obtained, for example, by cloning or gene synthesis. A polypeptide “derived from the soluble part of the glycoprotein of an enveloped virus” also designates a variant of a fragment, or a variant of a part of a glycoprotein contained in the envelope of a virus.

The term “glycoprotein” refers to a protein containing oligosaccharide chains covalently attached to polypeptide side-chains.

The expression “that interacts with a cell surface receptor” means that the polypeptide or glycoprotein is liable to recognize a receptor present on the surface of the cell. A polypeptide that interacts with a cell surface receptor can thus form a complex with said cell surface receptor.

In some embodiments, the polypeptide is soluble, i.e. it does not comprise a transmembrane domain. Therefore, in some embodiments of the invention, the polypeptide of the invention is a soluble polypeptide. As used herein, the term “soluble polypeptide” refers to a polypeptide which is not anchored within a membrane, such as, for example, by a transmembrane or a GPI anchor domain.

The polypeptide of the invention is a polypeptide of sequence SEQ ID NO: 1 or a functional variant thereof, which specifically binds to ASCT1 and does not specifically bind to ASCT2, wherein:

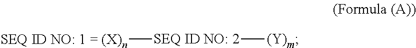

(X)ₙ is a sequence of n amino acids;
(Y)ₘ is a sequence of m amino acids;
n≥0 and m≥0; and
if m≥1 then (Y)ₘ does not comprise or consist of the m first amino acids of the sequence SEQ ID NO: 3 or a functional variant thereof, said functional variant of the m first amino acids of the sequence SEQ ID NO: 3 being such that SEQ ID NO: 2-(Y)ₘ specifically binds to ASCT2; and
   wherein the functional variant of SEQ ID NO: 1 has a sequence comprising a functional variant of SEQ ID NO: 2 instead of SEQ ID NO: 2, wherein said functional variant of SEQ ID NO: 2 specifically binds to ASCT1.

In some embodiments, SEQ ID NO: 1 consists of (X)ₙ-SEQ ID NO: 2-(Y)ₘ (Formula (A)).

In some embodiments, “—” designates a peptide bound.

In Formula (A), (X)ₙ is an amino acid sequence of n amino acids, and (Y)ₘ is an amino acid sequence of m amino acids. In Formula (A), the symbol “—” designates a peptide bound between the last amino acid of the sequence (X), and the first amino acid of the sequence SEQ ID NO: 2. In Formula (A), the symbol “—-” also designates a peptide bound between the last amino acid of the sequence SEQ ID NO: 2 and the first amino acid of the sequence (Y)ₘ.

In Formula (A), n=0 means that the sequence SEQ ID NO: 1 does not comprise any amino acid bound to the N terminal end of the sequence SEQ ID NO: 2. In Formula (A), n=0 may also mean that the functional variant of SEQ ID NO: 1 does not comprise any amino acid bound to the N terminal end of the functional variant of SEQ ID NO: 2.

In some embodiments, n=0 in Formula (A). Thus, in some embodiments, the sequence SEQ ID NO: 1 does not comprise any amino acid bound to the N terminal end of the sequence SEQ ID NO: 2. In some embodiments, the functional variant of SEQ ID NO: 1 does not comprise any amino acid bound to the N terminal end of the functional variant of SEQ ID NO: 2.

In Formula (A), m=0 means that the sequence SEQ ID NO: 1 does not comprise any amino acid bound to the C terminal end of the sequence SEQ ID NO: 2. In Formula (A), m=0 may also mean that the functional variant of SEQ ID NO: 1 does not comprise any amino acid bound to the C terminal end of the functional variant of SEQ ID NO: 2.

In some embodiments, m=0 in Formula (A). Thus, in some embodiments, the sequence SEQ ID NO: 1 does not comprise any amino acid bound to the C terminal end of the sequence SEQ ID NO: 2. In some embodiments, the functional variant of SEQ ID NO: 1 does not comprise any amino acid bound to the C terminal end of the functional variant of SEQ ID NO: 2.

In some embodiments, n=0 and m=0 in Formula (A). In those embodiments, the polypeptide of the invention is a polypeptide of sequence SEQ ID NO: 2 or a functional variant of SEQ ID NO: 2.

In Formula (A), n≥1 means that the sequence SEQ ID NO: 1 comprises an amino acid sequence of at least 1 amino acid bound to the N terminal end of the sequence SEQ ID NO: 2. In Formula (A), n≥1 may also mean that the functional variant of SEQ ID NO: 1 comprises an amino acid sequence of at least 1 amino acid bound to the N terminal end of the functional variant of SEQ ID NO: 2.

In Formula (A), m≥1 means that the sequence SEQ ID NO: 1 comprises an amino acid sequence of at least 1 amino acid bound to the C terminal end of the sequence SEQ ID NO: 2. In Formula (A), m≥1 may also mean that the functional variant of SEQ ID NO: 1 comprises an amino acid sequence of at least 1 amino acid bound to the C terminal end of the functional variant of SEQ ID NO: 2.

(X)ₙ may be any amino acid sequence of n amino acids.

In some embodiments, (X)ₙ comprises, or consists of, a signal peptide sequence. A “signal peptide”, also referred to as “signal sequence”, “targeting signal”, “localization signal”, “localization sequence”, “transit peptide”, “leader sequence” or “leader peptide” is a short peptide, e.g. 15-40 amino acids long, usually present at the N-terminus of most newly synthesized proteins that are destined toward the secretory pathway. A signal peptide may target the protein which comprises it for transfer to a specific organelle (such as the endoplasmic reticulum, Golgi or endosomes), or for insertion into a cellular membrane, or for secretion.

In some embodiments, (X)ₙ comprises, or consists of, the sequence of the SNV Env signal peptide (SEQ ID NO: 48). In some embodiments, (X), comprises, or consists of, the n first amino acids of the sequence SEQ ID NO: 13. In some embodiments, (X)ₙ comprises, or consists of, the 36 first amino acids of the sequence SEQ ID NO: 13.

Other examples of signal peptide sequences include, but are not limited to, human IL-2 signal peptide (SEQ ID NO: 28), human albumin signal peptide (SEQ ID NO: 29), human chymotrypsinogen signal peptide (SEQ ID NO: 30), human trypsinogen-2 signal peptide (SEQ ID NO: 31), Gaussia luciferase signal peptide (SEQ ID NO: 32), and mouse IgM signal peptide (SEQ ID NO: 33).

In some embodiments, (X)ₙ comprises, or consists of, a signal peptide sequence of SEQ ID NO: 28, SEQ ID NO: 29, SEQ ID NO: 30, SEQ ID NO: 31, SEQ ID NO: 32 or SEQ ID NO: 33.

(Y)ₘ may be any amino acid sequence of m amino acids except the m first amino acids of the sequence SEQ ID NO: 3. Also, (Y)ₘ may be any amino acid sequence of m amino acids except a functional variant of the m first amino acids of the sequence SEQ ID NO: 3.

A polypeptide of sequence SEQ ID NO: 2-(Y)ₘ wherein (Y)ₘ is the m first amino acids of the sequence SEQ ID NO: 3 is a polypeptide that binds to ASCT1 but also to ASCT2. Such a polypeptide is not specific of ASCT1 and is therefore not a polypeptide according to the invention.

As used herein, “a functional variant of the m first amino acids of the sequence SEQ ID NO: 3” is defined by the fact that the polypeptide of sequence SEQ ID NO: 2-(Y)ₘ, wherein (Y)ₘ is a functional variant of the m first amino acids of the sequence SEQ ID NO: 3, binds to ASCT1 but also to ASCT2. Such a polypeptide of sequence SEQ ID NO: 2-(Y)ₘ, wherein (Y)ₘ is a functional variant of the m first amino acids of the sequence SEQ ID NO: 3, is not specific of ASCT1 and is therefore not a polypeptide according to the invention.

In some embodiments, (Y)ₘ is an amino acid sequence corresponding to the m first amino acids of the sequence SEQ ID NO: 8 or a functional variant thereof.

In some embodiments, (Y)ₘ is an amino acid sequence corresponding to the m first amino acids of the sequence SEQ ID NO: 9 or a functional variant thereof.

In some embodiments, (Y)ₘ is an amino acid sequence corresponding to the m first amino acids of the sequence SEQ ID NO: 10 or a functional variant thereof.

In some embodiments, (Y)ₘ is an amino acid sequence corresponding to the m first amino acids of the sequence SEQ ID NO: 11 or a functional variant thereof.

In some embodiments, (Y)ₘ is an amino acid sequence corresponding to the m first amino acids of the sequence SEQ ID NO: 12 or a functional variant thereof.

A polypeptide “variant” as the term is used herein, is a polypeptide that typically differs from a polypeptide specifically disclosed herein in one or more substitutions, deletions, additions and/or insertions. Such variants may be naturally occurring or may be synthetically generated, for example, by modifying one or more of the above polypeptide sequences and evaluating one or more biological activities of the polypeptide as described herein and/or using any of a number of techniques well known in the art. Modifications may be made in the structure of polypeptides and still obtain a functional molecule that encodes a variant or derivative polypeptide with desirable characteristics.

When it is desired to alter the amino acid sequence of a polypeptide to create an equivalent, or even an improved, variant of a polypeptide of the invention, one skilled in the art will typically change one or more of the codons of the encoding DNA sequence. For example, certain amino acids may be substituted by other amino acids in a protein structure without appreciable loss of its ability to bind cell surface receptor, preferably cell surface nutrient transporters. Since it is the binding capacity and nature of a protein that defines that protein's biological functional activity, certain amino acid sequence substitutions can be made in a protein sequence, and, of course, its underlying DNA coding sequence, and nevertheless obtain a protein with similar properties. It is thus contemplated that various changes may be made in the peptide sequences, or corresponding DNA sequences that encode said peptides without appreciable loss of their biological utility or activity. In many instances, a polypeptide variant will contain one or more conservative substitutions. A “conservative substitution” is one in which an amino acid is substituted by another amino acid that has similar properties, such that one skilled in the art of peptide chemistry would expect the secondary structure and hydropathic nature of the polypeptide to be substantially unchanged. As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine. Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include histidine, lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) Ala, Pro, Gly, Glu, Asp, Gln, Asn, Ser, Thr; (2) Cys, Ser, Tyr, Thr; (3) Val, Ile, Leu, Met, Ala, Phe; (4) Lys, Arg, His; and (5) Phe, Tyr, Trp, His.

As used herein, the term “conservative amino acid substitution” may further be defined as an amino acid exchange within one of the following five groups:
I. Small aliphatic, nonpolar or slightly polar residues: Ala, Ser, Thr, Pro, Gly,
II. Polar, negatively charged residues and their amides: Asp, Asn, Glu, Gln,
III. Polar, positively charged residues: His, Arg, Lys,
IV. Large, aliphatic, nonpolar residues: Met, Leu, Ile, Val, Cys,
V. Large, aromatic residues: Phe, Tyr, Trp.

A variant may also, or alternatively, contain non-conservative changes. In a preferred embodiment, variant polypeptides differ from a native sequence by substitution, deletion or addition of 1, 2, 3, 4, 5, 6, 7, 8, 9 or 10 amino acids. Variants may also (or alternatively) be modified by, for example, the deletion or addition of amino acids that have minimal influence on the immunogenicity, secondary structure and hydropathic nature of the polypeptide.

In some embodiments, the variant is a functional variant. A “functional variant” of a polypeptide is a variant of said polypeptide which exhibits a same function or activity as said polypeptide, for example the same capacity of binding to a particular protein or receptor.

The functional variant of SEQ ID NO: 1 according to the invention has a sequence comprising a functional variant of SEQ ID NO: 2 instead of SEQ ID NO: 2 in Formula (A).

According to the invention, said functional variant of SEQ ID NO: 2 specifically binds to ASCT1 and does not bind to ASCT2.

In some embodiments, the functional variant of SEQ ID NO: 2 is capable of binding to ASCT1 with an affinity at least equivalent to the one of SEQ ID NO: 2.

In some embodiments, said functional variant of SEQ ID NO: 2 has the same length as SEQ ID NO: 2.

In some embodiments, the functional variant of the sequence SEQ ID NO: 1 has a sequence at least 70%, 75%, 80%, 85%, 90%, 91%, 92%, 93%, 94%, 95%, 96%, 97%, 98% or 99% identical to the sequence SEQ ID NO: 1.

In some embodiments, the functional variant of the polypeptide of sequence SEQ ID NO: 1 comprises or consists of an amino acid sequence presenting a sequence identity of at least about 75%, 80%, 85%, 90%, 91%, 92%, 93%, 94%, 95%, 96%, 97%, 98%, 99% with the amino acid sequence SEQ ID NO: 1.

Preferably, the functional variant of the sequence SEQ ID NO: 1 has a sequence at least 85%, preferably at least 90%, more preferably at least 95% identical to the sequence SEQ ID NO: 1.

In some embodiments, the length of the polypeptide of the invention or of the functional variant thereof is of less than 200, 199, 198, 197, 196, 195, 194, 193, 192, 191, 190, 189, 188, 187, 186, 185, 184, 183, 182, 181, 180, 179, 178, 177, 176, 175, 174, 173, 172, 171, 170, 169, 168, 167, 166, 165, 164, 163, 162, 161, 160, 159, 158, 157, 156, 155, 154, 153, 152, 151, 150, 149, 148, 147, 146, 145, 144, 143, 142, 141, 140, 139, 138, 137, 136, 135, 134, 133, 132, 131, 130, 129, 128, 127, 126, 125, 124, 123, 122, 121, 120, 119, 118, 117, 116, 115, 114, 113, 112, 111, 110, 109, 108, 107, 106, 105, 104, 103, 102, 101, 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60 amino acids.

Preferably, the length of the polypeptide of the invention or of the functional variant thereof is of less than 200 amino acids, less than 175 amino acids, less than 164 amino acids, less than 150 amino acids, less than 125 amino acids, less than 100 amino acids, less than 75 amino acids, less than 65 amino acids, less than 64 amino acids, or less than 60 amino acids.

More preferably, the length of the polypeptide of the invention or of the functional variant thereof is of less than 64 amino acids.

In some embodiments, the length of the polypeptide of the invention or of the functional variant thereof is of 63, 62, 61, 60 or 59 amino acids.

In some embodiments, the polypeptide of the invention or the functional variant thereof comprises or consists of amino acids 37 to 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199 of SEQ ID NO: 13.

In some embodiments, the polypeptide or functional variant thereof according to the invention has a sequence comprising or consisting of SEQ ID NO: 4.

In some embodiments, the polypeptide or functional variant thereof according to the invention has a sequence comprising or consisting of SEQ ID NO: 5.

In some embodiments, the polypeptide or functional variant thereof according to the invention has a sequence comprising or consisting of SEQ ID NO: 6.

In some embodiments, the polypeptide or functional variant thereof according to the invention has a sequence comprising or consisting of SEQ ID NO: 7.

In some embodiments, the polypeptide or functional variant thereof according to the invention has a sequence consisting of SEQ ID NO: 2.

The polypeptide of the invention or the functional variant thereof may be modified by means well-known in the art, for instance by the addition of one or more functional group such as a phosphate, acetate, lipid or carbohydrate group, and/or by the addition of one or more protecting group. For example, the polypeptide can be modified by the addition of one or more functional groups such as phosphate, acetate, or various lipids and carbohydrates. The polypeptide of the invention can also exist as a polypeptide derivative. The term “polypeptide derivative” refers to compound having an amino group (—NH—), and more particularly, a peptide bond. Polypeptides may be regarded as substituted amides. Like the amide group, the peptide bond shows a high degree of resonance stabilization. The C—N single bond in the peptide linkage has typically about 40 percent double-bond character and the C—O double bond about 40 percent single-bond character. “Protecting groups” are those groups that prevent undesirable reactions (such as proteolysis) involving unprotected functional groups. Specific examples of amino protecting groups include formyl; trifluoroacetyl; benzyloxycarbonyl; substituted benzyloxycarbonyl such as (ortho- or para-) chlorobenzyloxycarbonyl and (ortho- or para-) bromobenzyloxycarbonyl; and aliphatic oxycarbonyl such as t-butoxycarbonyl and t-amiloxycarbonyl. The carboxyl groups of amino acids can be protected through conversion into ester groups. The ester groups include benzyl esters, substituted benzyl esters such as methoxybenzyl ester; alkyl esters such as cyclohexyl ester, cycloheptyl ester or t-butyl ester. The guanidino moiety may be protected by nitro; or arylsulfonyl such as tosyl, methoxybenzensulfonyl or mesitylenesulfonyl, even though it does not need a protecting group. The protecting groups of imidazole include tosyl, benzyl and dinitrophenyl. The indole group of tryptophan may be protected by formyl or may not be protected.

The modification of the polypeptide of the invention may aim at improving its lifetime in vivo. One type of modification is the addition to the N- or C-termini of the polypeptide of polyethylene glycol (PEG). PEG is known by the person skilled in the art to have many properties that make it an ideal carrier for polypeptides such as high water-solubility, high mobility in solution and low immunogenicity. This modification also protects the polypeptides from exopeptidases and therefore increases their overall stability in vivo. Other modifications used to prevent degradation of the polypeptides by endopeptidases or exopeptidases include N-terminal modifications such as acetylation or glycosylation, C-terminal modifications such as amidation and use of unnatural amino acids (B-amino and cx-trifluoromethyl amino acids) at particular sites within the polypeptides. Another alternative to increase polypeptide molecular size is the genetic fusion of the polypeptide to the Fc domain of human immunoglobulin (including, for example, IgA, IgM and IgG) or the fusion of the polypeptide to albumin.

In some embodiments, the polypeptide or functional variant thereof according to the invention is glycosylated. In other embodiments, the polypeptide or functional variant thereof according to the invention is not glycosylated.

The polypeptide of the invention or the functional variant thereof specifically binds to ASCT1 and does not specifically bind to ASCT2.

The expression “specifically binds to”, as used herein, refers to the binding specificity and affinity of a molecule or a domain thereof for a particular target or epitope, or a domain thereof, even in the presence of a heterogeneous population of other proteins and biological molecules. Thus, in some embodiments, under designated assay conditions, the polypeptide of the invention binds preferentially to its target and does not bind in a significant amount to other components present in a test sample or subject. In some embodiments, such a polypeptide shows high affinity binding to its target with an equilibrium dissociation constant equal or below 1×10⁻⁶ M (e.g., at least 0.5×10⁻⁶, 1×10⁻⁷, 1×10⁻⁸, 1×10⁻⁹, 1×10⁻¹⁰ and less). Standard assays to evaluate the binding ability of two biological molecules are known in the art, including for example, ELISAs, Western blots, RIAs and flow cytometry. The binding kinetics (e.g., binding affinity) of the molecules also can be assessed by standard assays known in the art, such as by Biacore analysis.

Typically, when flow cytometry (FACS) is used to determine if a polypeptide of the invention or the functional variant thereof specifically binds to ASCT1, on cells naturally expressing ASCT1 or on ASCT1-transfected cells with a 100% transfection efficacy, the polypeptide of the invention or the functional variant thereof is typically considered to specifically bind to ASCT1 if at least 10%, 20%, 30%, 40%, 50%, 60%, 70%, 80%, 90% or 95% of the cells are positively bound and/or marked with the polypeptide of the invention or the functional variant thereof.

Typically, when flow cytometry (FACS) is used to determine if a polypeptide of the invention or the functional variant thereof specifically binds to ASCT1, on cells naturally expressing ASCT1 or on ASCT1-transfected cells with a 100% transfection efficacy, the polypeptide of the invention or the functional variant thereof is typically considered to specifically bind to ASCT1 if at least 10%, preferably 40%, even more preferably 70%, of the cells are positively bound and/or marked with the polypeptide of the invention or the functional variant thereof.

Alternatively, a polypeptide may be considered to specifically bind to ASCT1 if, when compared by FACS to a negative control (for instance the secondary antibody used in the FACS assay), on cells expressing ASCT1, the ratio of the MFI binding between the tested polypeptide over the negative control is higher than 5, 10, 20, 30, 40, 50, 100, 150, 200, 500, 1000 or 1500.

The expression “specifically binds to”, as used herein, also means that the polypeptide or functional variant of the invention recognize, bind and/or target ASCT1, but not ASCT2.

Typically, when flow cytometry (FACS) is used to determine if a polypeptide of the invention or the functional variant thereof specifically binds to ASCT2, on cells naturally expressing ASCT2 or on ASCT2-transfected cells with a 100% transfection efficacy, a polypeptide of the invention or the functional variant thereof is typically considered to not specifically bind to ASCT2 if at most 9%, preferably 5%, even more preferably 1%, of the cells are positively bound and/or marked with the polypeptide of the invention or the functional variant thereof.

In some embodiments, the polypeptide or variant of the invention is labeled with a detectable label or is coupled with a contrast agent.

The present invention thus also relates to a polypeptide or variant as described herein, wherein said polypeptide or variant is coupled to a detectable label or contrast agent.

Examples of detectable labels include, but are not limited to, radioactive labels, paramagnetic metals, fluorescents labels and peptidic tags.

In some embodiments, the polypeptide or variant of the invention is labeled with a radioactive label. Examples of radioactive labels include, but are not limited to, non-metallic radioisotopes and radioactive metals.

Examples of non-metallic radioisotopes comprise, but are not limited to, I-125, I-123, I-131, C-11, F-18, Br-75, Br-76, Br-77, Br-80, and At-211. The non-metallic radioisotopes may be conjugated covalently to either terminus of the polypeptide, functional groups of amino acid side chains, be part of a linear stabilized peptide as an additional substituent, e.g., in an amino acid phenylalanine or tyrosine carrying fluorine, bromine or iodine, or as an additional substituent carboxy or methyl, or as a replacement of any regular carbon atom in the polypeptide. These radioisotopes are useful in polypeptides as positron emission tomography (PET) probes or as single-photon emission computed tomography (SPECT) probes.

Examples of radioactive metals include, but are not limited to, Cu-64, Cu-67, Ga-67, Ga-68, Zr-89, Y-90, Tc-99m, In-111, Tb-161, Lu-177, Re-186, Re-188, and Bi-213. The radioactive metals may be covalently attached to the polypeptide, directly connected to the polypeptide or through a spacer.

In some embodiments, the polypeptide or variant of the invention is labeled with paramagnetic metals.

Examples of paramagnetic metals comprise, but are not limited to, Gd, Fe, Mn. The paramagnetic metals may be covalently attached to the polypeptides, directly connected to the polypeptides or through a spacer. These polypeptides are useful as magnetic resonance imaging (MRI) probes.

In some embodiments, the polypeptide or variant of the invention is labeled with a fluorescent label. Example of fluorescent label include, but are not limited to, fluorescent organic dyes, quantum dots and fluorescent protein. These polypeptides may be useful as optical imaging probes.

Example of fluorescent organic dyes include but are not limited to, commercial Alexa Fluor® dyes, fluorescein, rhodamine, or Cy® dyes (such as Cy3, Cy3.5, Cy5, Cy5.5, Cy7, and Cy7.5).

Example of fluorescent proteins include, but are not limited to, BFP, CFP, GFP, EGFP, mCherry, tdTomato, mPlum, mStrawberry, J-Red, DS-Red, mOrange, mCitrine, Venus, Ypet, YFP, Emerald, and the like. Another example of fluorescent protein is phycoerythrin. The fluorescent protein may be fused to the polypeptide by techniques of molecular cloning well known in the art.

In some embodiments, the polypeptide or variant of the invention is labeled with a peptidic tag.

Example of peptidic tags include, but are not limited to, an antibody crystallizable region (Fc), Enzymes (alkaline phosphatase or horseradish peroxidase), Hemagglutinin tag, Poly Arginine tag, Poly Histidine tag, Myc tag, Strep tag, S-tag, HAT tag, 3× Flag tag, Calmodulin-Binding Peptide tag, SBP tag, Chitin Binding Domain tag, GST tag, Maltose-Binding Protein tag, Fluorescent Protein tag, T7 tag, V5 tag, X-press tag and the like. The peptidic tag may be fused to the polypeptide by techniques of molecular cloning well known in the art or covalently attached to the polypeptide.

In some embodiments, the polypeptide or variant labeled as described herein is a fusion protein, wherein the polypeptide or variant is fused to a detection tag, such as, for example, a Fc fragment or a GFP. In some embodiments the polypeptide or variant of the invention is fused to a Fc fragment or a fluorescent protein. In some embodiments, the polypeptide or variant labeled as described herein is a fusion protein, wherein the polypeptide or variant is fused to phycoerythrin.

Examples of Fc fragments include, but are not limited to, rabbit Fc fragment (amino acid sequence SEQ ID NO: 24, encoded by SEQ ID NO: 25), mouse Fc fragment (amino acid sequence SEQ ID NO: 26, encoded by SEQ ID NO: 27).

In some embodiments, the polypeptide or variant of the invention, is fused to a Fc fragment.

In some embodiments, the polypeptide or variant of the invention is fused to a Fc fragment and does not comprise a signal peptide.

In some embodiments, the polypeptide or variant of the invention is fused to a Fc fragment and comprises a signal peptide.

In some embodiments, the polypeptide or variant of the invention is coupled with at least one detectable label. Non-limiting examples of contrast agents are listed herein. In some embodiments, the contrast agent is I-125.

In some embodiments, the polypeptide or variant of the invention is coupled with at least one detectable label, or with at least one contrast agent, or may be used as a probe for medical imaging.

As used herein, the term “contrast agent” refers to an agent used to improve the visibility of internal bodily structures in medical imaging technics.

The term “medical imaging” as used herein refers to imaging techniques suitable to visualize in vivo a subject's internal structures (i.e. tissues or organs). Such techniques include but are not limited to, computed tomography (CT scan), endoscopic ultrasound (EUS), magnetic resonance imaging (MRI), positron-emission tomography (PET), single photon emission tomography (SPECT), magnetic resonance cholangiopancreatography, fluorimetry, fluorescence, and near-infrared (NIR) fluorescent imaging. In the context of the invention the polypeptide or variant of the invention coupled with at least one detectable label may be used as a probe, to localize in vivo ASCT1-expressing cells in a subject's internal structures.

In some embodiments of the invention, the polypeptide or variant of the invention coupled with at least one detectable label is for use as a tracer. The present invention thus further relates to the use as a tracer of a polypeptide or variant thereof coupled with at least one detectable label. The term “tracer”, as used herein, refers to a recognition agent providing insight into ASCT1-associated disease location, progression and/or structure for pre-, intra- and post-operative surgery.

Methods for coupling at least one detectable label to a polypeptide are well known in the state of the art. For instance, the at least one detectable label may be bound covalently or non-covalently.

Techniques to couple polypeptides to I-125 are well known in the state of the art. A non-limited example of such a method is the following: iodine present in a reduced form (NaI) reacts with the phenol group of a tyrosine or with the side chain of a histidine residue. These groups are pre-oxidized with an oxidizing agent (iodogen). The peptides preparation (100 μg for 1 mCi=37 MBq) is then added to an iodogen solution and incubated for 10 minutes at 4° C. The reaction is stopped using a stop solution comprising for example 200 μL of PBS with sodium azide per marking. In parallel, a mouse serum is added onto a PD10 column. Then the reaction solution is added onto the PD10 column and the peptide coupled with the iodine is collected.

In embodiments concerning detectable labels encoded by a nucleic acid sequence, the detectable label may be fused to the polypeptide or variant of the invention, by techniques of molecular cloning well known in the art.

The invention further relates to a nucleic acid encoding the polypeptide or functional variant thereof according to the invention.

In some embodiments, the nucleic acid sequence encoding the polypeptide, or functional variant of the invention, comprises or consists of a nucleic acid sequence encoding the amino acid sequence of SEQ ID NO: 2 or a functional variant thereof.

In some embodiments, the nucleic acid sequence encoding the amino acid sequence of SEQ ID NO: 2 consists of the nucleic acid sequence of SEQ ID NO: 14.

In some embodiments, the nucleic acid sequence encoding the amino acid sequence of SEQ ID NO: 4 consists of the nucleic acid sequence of SEQ ID NO: 15.

In some embodiments, the nucleic acid sequence encoding the amino acid sequence of SEQ ID NO: 5 consists of the nucleic acid sequence of SEQ ID NO: 16.

In some embodiments, the nucleic acid sequence encoding the amino acid sequence of SEQ ID NO: 6 consists of the nucleic acid sequence of SEQ ID NO: 17.

In some embodiments, the nucleic acid sequence encoding the amino acid sequence of SEQ ID NO: 7 consists of the nucleic acid sequence of SEQ ID NO: 18.

In some embodiments, the nucleic acid sequence encoding the amino acid sequence of SEQ ID NO: 2, SEQ ID NO: 4, SEQ ID NO: 5, SEQ ID NO: 6, or SEQ ID NO: 7 or a functional variant thereof consists of a nucleic acid sequence presenting a sequence identity of at least 75%, 80%, 85%, 90%, 91%, 92%, 93%, 94%, 95%, 96%, 97%, 98%, 99% with the nucleic acid sequence SEQ ID NO: 14, SEQ ID NO: 15, SEQ ID NO: 16, SEQ ID NO: 17, or SEQ ID NO: 18.

The invention also pertains to an expression vector comprising a nucleic acid encoding the polypeptide or functional variant thereof according to the invention.

The invention further relates to a cell comprising a nucleic acid encoding the polypeptide or functional variant thereof according to the invention, or an expression vector comprising a nucleic acid encoding the polypeptide or functional variant thereof according to the invention.

The polypeptide described herein can be produced synthetically by chemical synthesis or enzymatic synthesis as it is well known in the art. Alternatively, nucleotide sequences encoding the polypeptide of the invention can be introduced into a protein expression vector and produced in a suitable host organism (e.g., bacteria, insect cells, etc.), then purified. In some embodiments, the polypeptide is obtained by a cloning method, such as, for example, using any production system known in the art, such as, for example, E. coli, yeast, baculovirus-insect cell, or mammalian cells such as HEK or CHO expression system.

An additional polypeptide (“tag”) can be added on for the purpose of purifying or identifying or purifying the polypeptide. Protein tags make it possible, for example, for the polypeptide to be adsorbed, with high affinity, to a matrix, and for the matrix then to be washed stringently with suitable buffers without the complex being eluted to any significant extent, and for the adsorbed complex subsequently to be eluted selectively. Examples of protein tags which are known to the skilled person are a (His) 6 tag, a Myc tag, a FLAG tag, a hemagglutinin tag, a glutathione transferase (GST) tag, intein having an affinity chitin-binding tag or maltose-binding protein (MBP) tag. These protein tags can be located N-terminally, C-terminally and/or internally.

In some embodiments, the sequence of the polypeptide is fused in N-terminal to a signal peptide sequence allowing the secretion of said polypeptide. Examples of signal peptide sequences include, but are not limited to, the SNV envelope glycoprotein signal peptide (SEQ ID NO: 48), human IL-2 signal peptide (SEQ ID NO: 28), human albumin signal peptide (SEQ ID NO: 29), human chymotrypsinogen signal peptide (SEQ ID NO: 30), human trypsinogen-2 signal peptide (SEQ ID NO: 31), Gaussia luciferase signal peptide (SEQ ID NO: 32), and mouse IgM signal peptide (SEQ ID NO: 33).

The present invention also relates to a composition comprising, consisting essentially of, or consisting of at least one polypeptide or functional variant thereof, nucleic acid, expression vector or cell according to the invention.

The present invention also relates to a pharmaceutical composition comprising, consisting essentially of, or consisting of at least one polypeptide or functional variant thereof, nucleic acid, expression vector or cell according to the invention, and at least one pharmaceutically acceptable excipient.

The present invention also relates to a diagnostic composition comprising, consisting essentially of, or consisting of at least one polypeptide or functional variant thereof, nucleic acid, expression vector or cell according to the invention, and at least one pharmaceutically acceptable excipient.

As used herein, the term “consisting essentially of”, with reference to a composition, means that the at least one polypeptide, or functional variant thereof, nucleic acid, expression vector or cell of the invention is the only one agent, therapeutic agent or diagnostic agent, with a biologic activity within said composition.

The present invention also relates to a diagnostic composition comprising or consisting essentially of at least one labeled polypeptide or functional variant thereof of the invention, and at least one pharmaceutically acceptable excipient.

As used herein, the term “pharmaceutically acceptable” refers to molecular entities and compositions that do not produce an adverse, allergic or other untoward reaction when administered to a subject, especially a human, as appropriate. Hence, “Pharmaceutically acceptable excipient” refers to molecular entities and compositions that do not produce an adverse, allergic or other untoward reaction when administered to a subject, especially a human, as appropriate. It includes any and all solvents, dispersion media, coatings, antibacterial and antifungal agents, isotonic and absorption delaying agents and the like. For human administration, preparations should meet, pyrogenicity, sterility, general safety and purity standards as required by regulatory offices, such as, for example, FDA Office or EMA. A pharmaceutically acceptable carrier or excipient may thus refer to a non-toxic solid, semi-solid or liquid filler, diluent, encapsulating material or formulation auxiliary of any type.

Pharmaceutically acceptable excipients include water, saline, Ringer's solution, dextrose solution, and solutions of ethanol, glucose, sucrose, dextran, mannose, mannitol, sorbitol, polyethylene glycol (PEG), phosphate, acetate, gelatin, collagen, Carbopol®, vegetable oils, and the like. One may additionally include suitable preservatives, stabilizers, antioxidants, antimicrobials, and buffering agents, such as, for example, BHA, BHT, citric acid, ascorbic acid, tetracycline, and the like.

Other examples of pharmaceutically acceptable excipients that may be used in the composition of the invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

In addition, pharmaceutically acceptable excipients may comprise some excipients, such as, for example, surfactants (e.g. hydroxypropylcellulose); suitable carriers, such as, for example, solvents and dispersion media containing, for example, water, ethanol, polyol (e.g. glycerol, propylene glycol, and liquid polyethylene glycol, and the like), suitable mixtures thereof, and vegetable oils, such as, for example, peanut oil and sesame oil; isotonic agents, such as, for example, sugars or sodium chloride; coating agents, such as, for example, lecithin; agents delaying absorption, such as, for example, aluminum monostearate and gelatin; preservatives, such as, for example, benzalkonium chloride, benzethonium chloride, chlorobutanol, thimerosal and the like; buffers, such as, for example, boric acid, sodium and potassium bicarbonate, sodium and potassium borates, sodium and potassium carbonate, sodium acetate, sodium biphosphate and the like; tonicity agents, such as, for example, dextrose, potassium chloride, propylene glycol, sodium chloride; antioxidants and stabilizers, such as, for example, sodium bisulfite, sodium metabisulfite, sodium thiosulfite, thiourea and the like; nonionic wetting or clarifying agents, such as, for example, polysorbate 80, polysorbate 20, poloxamer 282 and tyloxapol; viscosity modifying agents, such as, for example dextran 40, dextran 70, gelatin, glycerin, hydroxyethylcellulose, hydroxymethylpropylcellulose, lanolin, methylcellulose, petrolatum, polyethylene glycol, polyvinyl alcohol, polyvinylpyrrolidone, carboxymethylcellulose; and the like.

In some embodiments, the diagnostic composition of the invention is for diagnosing an ASCT1-associated disease, using a method of the invention as described hereinabove.

In some embodiments, the polypeptide or functional variant thereof, nucleic acid, expression vector or cell according to the invention is encapsulated.

The techniques of encapsulation are well known in the state of the art. Examples of capsule include, but are not limited to, phospholipids, polymers and liposomes.

In some embodiments, the polypeptide or functional variant thereof, nucleic acid, expression vector or cell according to the invention is encapsulated with a detectable label.

The present invention also relates to a medicament composition comprising, consisting essentially of, or consisting of at least one polypeptide or functional variant thereof, nucleic acid, expression vector or cell according to the invention.

Another object of the present invention is a kit for implementing the methods of the invention, wherein said kit comprises at least one polypeptide or functional variant thereof, nucleic acid, expression vector or cell according to the invention.

In some embodiments, the kit of the invention further comprises cells displaying ASCT1 at the cell surface for use as a reference.

The invention further relates to an in vitro method of specifically detecting or/and measuring the level of ASCT1 in a sample, while not detecting or/and measuring the level of ASCT2, wherein said method comprises the steps of:
a) contacting said sample with the polypeptide or functional variant thereof according to the invention, and
b) detecting and/or measuring the binding of said polypeptide or functional variant thereof to ASCT1.

The invention further relates to the polypeptide or functional variant thereof according to the invention, for use for specifically detecting or/and measuring the level of ASCT1 in vivo, while not detecting or/and measuring the level of ASCT2.

In some embodiments, the term “level of ASCT1” refers to the amount of ASCT1 present at the surface of a cell and/or within the cell.

In some embodiments, the method of the invention is for assessing the expression level of ASCT1 present on the cell surface. In other embodiments, the method of the invention is for assessing the expression level of ASCT1 present within the cell.

In some embodiments, the in vitro method of the invention is for diagnosing or monitoring an ASCT1-associated disease in a subject.

In some embodiments, the sample is a biological sample.

Examples of biological samples include, but are not limited to, body fluids, cell samples, tissue samples, biopsy samples.

In some embodiments, the biological sample is a body fluid. Examples of body fluids include, but are not limited to, blood, plasma, serum, lymph, ascetic fluid, cystic fluid, urine, bile, synovial fluid, bronchoalveolar lavage fluid, sputum, amniotic fluid, peritoneal fluid, cerebrospinal fluid, pleural fluid, pericardial fluid, semen, saliva, sweat and milk.

In some embodiments the biological sample is a tissue sample. Examples of tissues include, but are not limited to, placenta, intestine, brain, liver, lung, kidney, cornea, retina, heart breast, cervix, kidney, pancreas, ovary, skin, nerve, spleen, thymus, esophagus, stomach, testis, hair, skin, bone, uterus, bladder and spinal cord.

In some embodiments, the biological sample is a biopsy sample. In some embodiments, the biological sample is a fine-needle aspirate sample. In some embodiments, the biological sample is a resection sample.

In some embodiments, the biological sample is a cell sample. Examples of cell samples include, without being limited to, red blood cells, peripheral blood mononuclear cells (PBMC), peripheral white blood cells, cell samples obtained from tissue biopsies such as lymph nodes biopsies, intestinal or synovial biopsies, or cell sample obtained from broncho-alveolar lavage or cerebrospinal fluid, cell culture sample.

In some embodiments, the methods according to the present invention comprise a step of providing a biological sample from a subject.

In some embodiments, the sample was previously taken from the subject, i.e., the in vitro methods of the invention do not comprise a step of recovering a sample from the subject. Consequently, according to this embodiment, the in vitro methods of the invention are non-invasive methods.

The methods of the invention comprise the step of detecting and/or measuring the binding of a polypeptide of the invention, to ASCT1 in a sample.

Techniques to measure the binding of a polypeptide, or of a ligand, to its receptor are known in the art and may imply the detection and measure of the amount of the ligand-receptor complexes. In the context of the present invention, such techniques could for example rely on the detection and measure of the number of complexes formed by the polypeptide of the invention with ASCT1 present in the sample, such as, for example, at the cell surface of cells present in a biological sample.

Example of such technique include, but are not limited to, flow cytometry analysis, immunohistochemistry, western blot associated or not with cell fractionation, enzyme-linked immunosorbent assay (ELISA), sandwich ELISA, fluorescent-linked immunosorbent assay (FLISA), enzyme immunoassay (EIA), radioimmunoassay (RIA), image analysis, for example high content analysis, computed tomography (CT scan), endoscopic ultrasound (EUS), magnetic resonance imaging (MRI), positron-emission tomography (PET), single photon emission tomography (SPECT), magnetic resonance cholangiopancreatography, fluorimetry, fluorescence, and near-infrared (NIR) fluorescent imaging and the like.

Examples of such techniques amenable to an in vitro use include, but are not limited to, immunohistochemistry, Multiplex methods (Luminex), western blot, enzyme-linked immunosorbent assay (ELISA), sandwich ELISA, fluorescent-linked immunosorbent assay (FLISA), enzyme immunoassay (EIA), radioimmunoassay (RIA), flow cytometry (FACS), and the like.

Examples of such techniques suitable for an in vivo use include, but are not limited to, computed tomography (CT scan), endoscopic ultrasound (EUS), magnetic resonance imaging (MRI), positron-emission tomography (PET), single photon emission tomography (SPECT), magnetic resonance cholangiopancreatography, fluorimetry, fluorescence, and near-infrared (NIR) fluorescent imaging.

The present invention also relates to an in vivo method for specifically detecting or/and measuring the level of ASCT1, using a polypeptide or variant thereof according to the invention, while not detecting or/and measuring the level of ASCT2.

The present invention also relates to a polypeptide or variant thereof according to the invention, for use in an in vivo method for specifically detecting or/and measuring the level of ASCT1 in a subject, while not detecting or/and measuring the level of ASCT2.

The present invention also relates to the use of a polypeptide or variant thereof according to the invention, for the manufacture of a kit for specifically detecting or/and measuring the level of ASCT1 in a subject, while not detecting or/and measuring the level of ASCT2.

In some embodiments, said method comprises the detection and/or measure of the level of ASCT1 within the body of a subject, such as, for example, in a specific organ or tissue.

The present application also relates to an in vivo method for specifically detecting or/and measuring the level of ASCT1 in a subject, while not detecting or/and measuring the level of ASCT2, comprising:
a) contacting a polypeptide or variant thereof according to the invention with a cell, a sample, a tissue or an organ, and
b) detecting and/or quantifying the polypeptide or variant thereof bound to ASCT1 present in the cell, sample, tissue or organ within said subject.

The present invention also relates to a polypeptide or variant thereof according to the invention, for use in an in vivo method for specifically detecting or/and measuring the level of ASCT1 in a subject, while not detecting or/and measuring the level of ASCT2, comprising:
a) contacting a polypeptide or variant thereof according to the invention with a cell, a sample, a tissue or an organ, and
b) detecting and/or quantifying the polypeptide or variant thereof bound to ASCT1 present in the cell, sample, tissue or organ within said subject.

The present application also relates to an in vivo method for specifically detecting or/and measuring the level of ASCT1 in a subject, while not detecting or/and measuring the level of ASCT2, comprising:
a) administering to the subject a polypeptide or variant thereof according to the invention, and
b) detecting and/or quantifying the binding of said polypeptide or variant thereof to ASCT1 within said subject, for example by medical imaging.

The present invention also relates to a polypeptide or variant thereof according to the invention, for use in an in vivo method for specifically detecting or/and measuring the level of ASCT1 in a subject, while not detecting or/and measuring the level of ASCT2, comprising:
a) administering to the subject a polypeptide or variant thereof according to the invention, and
b) detecting and/or quantifying the binding of said polypeptide or variant thereof to ASCT1 within said subject, for example by medical imaging.

In some embodiments, the polypeptide or variant according to the invention is coupled with at least one detectable label, and may be used for in vivo diagnosis by medical imaging.

In some embodiments, the polypeptide or variant administered to the subject is comprised in a composition, preferably a pharmaceutical composition or a diagnostic composition.

In some embodiments, said method comprises the detection and/or measure of the level of ASCT1 using medical imaging techniques.

Examples of specific medical imaging techniques that may be used are well known to the skilled artisan and include, but are not limited to, computer assisted tomography (CAT), magnetic resonance spectroscopy (MRS), magnetic resonance imaging (MRI), positron emission tomography (PET) or single-photon emission computed tomography (SPECT) and are described in Boonstra et al. (2015. Oncotarget. 6 (16): 14260-73).

The present invention thus relates to an in vivo method for specifically detecting or/and measuring the level of ASCT1 in a subject, while not detecting or/and measuring the level of ASCT2, comprising the steps of:
a) administering to said subject a labeled polypeptide or variant thereof according to the invention; and,
b) detecting and/or measuring the binding of said labeled polypeptide or variant thereof to ASCT1 using medical imaging.

The present invention also relates to a polypeptide or variant thereof according to the invention, for use in an in vivo method for specifically detecting or/and measuring the level of ASCT1 in a subject, while not detecting or/and measuring the level of ASCT2, comprising the steps of:
a) administering to said subject a labeled polypeptide or variant thereof according to the invention; and,
b) detecting and/or measuring the binding of said labeled polypeptide or variant thereof to ASCT1 using medical imaging.

In some embodiments, the methods of the invention are for diagnosing or monitoring an ASCT1-associated disease in a subject.

In some embodiments, the methods of the present invention are for identifying a subject as presenting a risk of developing an ASCT1-associated disease.

In some embodiments, the methods of the invention are for assessing the severity of an ASCT1-associated disease in a subject, or for prognosing an ASCT1-associated disease.

Therefore, the present invention relates to a method for diagnosing an ASCT1-associated disease in a subject, for identifying a subject as presenting a risk of developing an ASCT1-associated disease, for assessing the severity of an ASCT1-associated disease in a subject, for prognosing an ASCT1-associated disease in a subject, or for monitoring an ASCT1-associated disease in a subject, said method comprising specifically detecting or/and measuring the level of ASCT1, using a polypeptide or variant thereof according to the invention.

The present invention also relates to a polypeptide or variant thereof according to the invention, for use in a method of diagnosing an ASCT1-associated disease in a subject, identifying a subject as presenting a risk of developing an ASCT1-associated disease, assessing the severity of an ASCT1-associated disease in a subject, prognosing an ASCT1-associated disease in a subject, or monitoring an ASCT1-associated disease in a subject, said method comprising detecting or/and measuring the level of ASCT1, using a polypeptide or variant thereof according to the invention.

The present invention also relates to the use of a polypeptide or variant thereof according to the invention for detecting or/and measuring the level of ASCT1, in the manufacture of a kit for the diagnosis, prognosis or monitoring of an ASCT1-associated disease.

The present invention thus also relates to a method for diagnosing an ASCT1-associated disease in a subject, for identifying a subject as being at risk of developing an ASCT1-associated disease, for assessing the severity of an ASCT1-associated disease in a subject, for prognosing an ASCT1-associated disease in a subject, or for monitoring an ASCT1-associated disease in a subject, wherein said method comprises the steps of:
a) contacting a biological sample from said subject with a polypeptide or variant thereof according to the invention; and
b) measuring the binding of said polypeptide or variant thereof to ASCT1.

The present invention also relates to a polypeptide or variant thereof according to the invention, for use in a method of diagnosing an ASCT1-associated disease in a subject, identifying a subject as presenting a risk of developing an ASCT1-associated disease, assessing the severity of an ASCT1-associated disease in a subject, prognosing an ASCT1-associated disease in a subject, or monitoring an ASCT1-associated disease in a subject, wherein said method comprises the steps of:
a) contacting a biological sample from said subject with a polypeptide or variant thereof according to the invention; and
b) measuring the binding of said polypeptide or variant thereof to ASCT1.

The present application also relates to a method for diagnosing an ASCT1-associated disease, for identifying a subject as being at risk of developing an ASCT1-associated disease, for assessing the severity of an ASCT1-associated disease in a subject, for prognosing an ASCT1-associated disease in a subject, or for monitoring an ASCT1-associated disease in a subject, said method comprising the steps of:
a) contacting the polypeptide or variant thereof according to the invention to a cell, sample, tissue, and/or organ,
b) detecting and/or measuring the binding of said polypeptide or variant to ASCT1 in said cell, sample, tissue, and/or organ.

The present invention also relates to a polypeptide or variant thereof according to the invention, for use in a method of diagnosing an ASCT1-associated disease in a subject, identifying a subject as presenting a risk of developing an ASCT1-associated disease, assessing the severity of an ASCT1-associated disease in a subject, prognosing an ASCT1-associated disease in a subject, or monitoring an ASCT1-associated disease in a subject, said method comprising the steps of:
a) contacting the polypeptide or variant thereof according to the invention to a cell, sample, tissue, and/or organ,
b) detecting and/or measuring the binding of said polypeptide or variant to ASCT1 in said cell, sample, tissue, and/or organ.

In some embodiments, said method does not comprise detecting or/and measuring the level of ASCT2.

The term “ASCT1-associated disease” as used herein refers to diseases wherein pathways involving serine, alanine and/or cysteine homeostasis and/or metabolism are dysregulated.

In some embodiments, the ASCT1-associated disease is a disorder related to a mutation within the SLC1A4 gene. In some embodiments, said mutation within the SLC1A4 gene results in a partial or complete loss of function or partial or complete loss of expression.

In some embodiments, the ASCT1-associated disease is a disorder related to a mutation within the SLC1A4 gene encoding a mutated protein ASCT1 comprising for example one of the following mutations: E256K, L315fs in particular L315Hfs*42, G381R or R457W. These mutations are notably associated to intellectual disability, microcephaly, spasticity and thin corpus callosum and progressive microcephaly (SPATCCM), developmental delay, hypomyelination, epileptic encephalopathy and/or severe hypotonia.

In some embodiments, the ASCT1-associated disease is cancer, a neurological disease, a neurodegenerative disease, a disorder of pregnancy or a neuroinflammatory disease.

Examples of ASCT1-associated diseases include, but are not limited to, serine-associated cancers, alanine-associated cancers, cysteine-associated cancers.

Examples of ASCT1-associated cancers include, but are not limited to, tumors, sarcomas, carcinomas, leukemias, lymphomas, and metastasis.

Preferably, the ASCT1-associated cancer is gastrointestinal cancer, digestive tract cancer, prostate cancer, pancreas cancer, liver cancer, breast cancer, lymphoma, brain cancer, skin cancer, colon cancer, colorectal cancer, lung cancer, kidney cancer, parotid cancer, or ovarian cancer. The ASCT1-associated cancer may also be gallbladder cancer, thyroid cancer, urothelial cancer, cervical cancer or endometrial cancer.

Preferably, the ASCT1-associated cancer is esophageal adenocarcinoma, prostate adenocarcinoma, pancreatic ductal adenocarcinoma, hepatocellular carcinoma, micropapillary carcinoma, diffuse large B-cell lymphoma (DLBCL), neuroblastoma, liposarcoma, melanoma, colorectal adenocarcinoma, renal cell carcinoma, salivary gland carcinoma, ovarian carcinoma.

Examples of ASCT1-associated diseases include, but are not limited to, schizophrenia, visual dysfunction, amyotrophic lateral sclerosis (ALS), microcephaly, SPATCCM (spasticity and thin corpus callosum and progressive microcephaly), cardiovascular diseases (CVD), ischemic stroke, hereditary sensory neuropathy type 1, central nervous system injury, neurological disorders, autism spectrum disorder (ASD), dementia, Alzheimer's disease, Parkinson's disease, Huntington's disease, epilepsy, diabetes including e.g. diabetic neuropathy and gestational diabetes mellitus, renal dysfunction-linked conditions including e.g. chronic renal diseases and renal cell carcinoma, vitiligo and cancers including e.g. ASCT1-associated cancers.

Preferably, the ASCT1-associated disease is schizophrenia, visual dysfunction, amyotrophic lateral sclerosis (ALS), microcephaly in children disorder, and SPATCCM (spasticity and thin corpus callosum and progressive microcephaly).

Examples of metabolic diseases include, but are not limited to, obesity, diabetes, cardiovascular mortality, renal damage and ischemia.

Examples of inflammatory diseases include, but are not limited to, polyarthritis, rheumatoid arthritis, asthma, inflammatory bowel diseases, celiac diseases, autoimmune diseases and multiple sclerosis.

In some embodiments, the ASCT1-associated disease is not pancreatic cancer.

In some embodiments, the methods of the invention comprise a step of comparing the binding detected and/or measured at step b) with a reference binding value.

As used herein, the term “reference” broadly encompasses any suitable reference binding level which may be used as a basis for comparison with respect to the determined binding. In some embodiments, the reference is constructed using algorithms and/or other methods of statistical and hierarchical classification. In another aspect, the reference binding level is stored in a database to provide a stored binding level and the stored binding level is used to determine the difference in the binding level. The database may, for example, be stored on a computer or a server.

In some embodiments, the reference binding level is an index value or is derived from one or more risk prediction algorithms or computed indices for the presence of cells wherein the function of ASCT1 is altered (e.g., increased or decreased). A reference binding level can be relative to a number or value derived from population studies, including without limitation, such populations of subjects having similar age range, subjects in the same or similar ethnic group.

The term “cells wherein the function of ASCT1 is altered” as used herein refers to cells wherein serine, alanine and/or cysteine metabolism or influx is abnormally increased or decreased.

Serine metabolism include its synthesis, catabolism but also dietary uptake. Serine deficiency is associated to primary disorders of serine metabolism, in particular serine biosynthesis. Serine, and in particular L-serine, deficiency or decrease is also associated with impaired function of the nervous system. For example, serine, in particular L-serine, deficiency or decrease is associated to hereditary sensory neuropathy type 1, central nervous system injury, and to a wide range of neurological and psychiatric disorders including Alzheimer's disease, Parkinson's disease, amyotrophic lateral sclerosis, Huntington's disease, and schizophrenia. Serine, in particular L-serine, deficiency or decrease is also associated to hyperhomocysteinemia, diabetic neuropathy, and chronic renal diseases.

Alanine metabolism include its synthesis, catabolism but also dietary uptake. D-alanine is involved in various neurological and psychiatric disorders including schizophrenia, Alzheimer's disease, and many other diseases such as renal diseases, diabetes and cancers.

Cysteine metabolism include its synthesis, catabolism but also dietary uptake. Hyperhomocysteinemia (elevated levels of homocysteine) is considered as toxic for cells and is associated with different health problems. For example, hyperhomocysteinemia and low levels of cysteine are associated with various diseases such as cardiovascular diseases (CVD), ischemic stroke, neurological disorders, autism spectrum disorder (ASD), dementia, Alzheimer's disease, epilepsy, diabetes including e.g. diabetic neuropathy and gestational diabetes mellitus, cancers including e.g. lung cancer, colorectal cancer and digestive tract cancer, renal dysfunction-linked conditions including e.g. renal cell carcinoma, and vitiligo.

In some embodiments, the reference value is determined by measuring the binding of the polypeptide of the invention, or variant thereof, to ASCT1 in a reference population.

In some embodiments, the reference population refers to a population comprising at least 1, preferably at least 5, at least 10, at least 15, at least 20, at least 25, at least 30, at least 35, at least 40, at least 45, at least 50, at least 75, at least 100 or more substantially healthy subjects, i.e., subject who are not affected and/or who have not been diagnosed with the ASCT1-associated disease being considered. According to those embodiments, a determined binding level different from the reference binding level may be indicative of the presence of an ASCT1-associated disease.

In other embodiments, the reference population refers to a population comprising at least 1, preferably at least 5, at least 10, at least 15, at least 20, at least 25, at least 30, at least 35, at least 40, at least 45, at least 50, at least 75, at least 100 or more subjects affected (and preferably diagnosed) with the ASCT1-associated disease being considered. According to those embodiments, a determined binding level different from the reference binding level may be indicative of the absence of an ASCT1-associated disease.

In the present invention, two numeric values, in particular two binding levels, are considered as different if the first numeric value is higher (such as, for example, the first numeric value is about 20% higher than the second one, preferably is about 30, 40, 50, 60, 70, 80, 90% or more higher than the second one) or lower than the second one (such as, for example, the second numeric value is about 20% lower than the second one, preferably is about 30, 40, 50, 60, 70, 80, 90% or more lower than the second one).

In some embodiments, two numeric values, in particular two binding levels, are considered as different if the first numeric value is increased by a factor of or above about 1.01, preferably by a factor of or above, about 1.02, 1.03, 1.04, 1.05, 1.06, 1.07, 1.08, 1.09, more preferably the value is increased by a factor of or above about 1.1, 1.15, 1.20, 1.25, 1.30, 1.3, 1.35, 1.40, 1.45, 1.50, 1.55, 1.60, 1.65, 1.70, 1.75, 1.80, 1.85, 1.90, 1.95, more preferably by a factor of or above about 2, 3, 4, 5 or more when compared to the second value or if the first numeric value is decreased by a factor of or below about 0.99, preferably by a factor of or below, about 0.98, 0.97, 0.96, 0.95, 0.94, 0.93, 0.92, 0.91, more preferably the value is decreased by a factor of or below about 0.90, 0.85, 0.80, 0.75, 0.70, 0.65, 0.60, 0.55, 0.50, 0.45, 0.40, 0.35, 0.30, 0.25, 0.20, 0.15, 0.1 or less when compared to the second value.

In some embodiments, by implying a multitude of measures of the binding of the polypeptide of the invention to ASCT1 in the reference population, it is conceivable to use as reference value a mathematical representation of the binding of the polypeptide of the invention to ASCT1 such as for example, a mean or a median.

In some embodiments, the reference value is a personalized reference, determined at different time points in the same subject (such as, for example, before receiving a treatment for an ASCT1-associated disease).

In some embodiments, the reference value is an internal reference value, determined in different part of the same subject, such as, for example, in different organs or tissues. This type of reference value is in particular useful in the implementation of method based on medical imaging.

The present invention thus relates to an in vitro method for diagnosing a subject with, or identifying a subject as being at risk of developing an ASCT1-associated disease, wherein said method comprises the steps of:
a) contacting a biological sample previously obtained from said subject with a polypeptide or variant thereof according to the invention;
b) measuring the binding of said polypeptide or a variant thereof to ASCT1; and
c) comparing the binding measured at step b) with a reference value.

In some embodiments, the step of comparing the binding of the polypeptide or variant of the invention to a reference value allows to diagnose a subject with, or at risk of developing, an ASCT1-associated disease.

In some embodiments, the method of the invention is for diagnosing or for assessing a risk of developing an ASCT1-associated disease associated with an increased ASCT1 level in a subject, and a binding of the polypeptide or variant of the invention to ASCT1 measured at step b) higher than the binding of the polypeptide or variant of the invention to ASCT1 measured in a reference population of substantially healthy subjects (or in a reference sample from a reference population) is indicative of the presence of said disease, or of a risk of developing said disease.

In some embodiments, the method of the invention is for diagnosing or for assessing a risk of developing an ASCT1-associated disease associated with an increased ASCT1 level at the cell surface in a subject, and a binding of the polypeptide or variant of the invention to ASCT1 measured at step b) higher than the binding of the polypeptide or variant of the invention to ASCT1 measured in a reference population of substantially healthy subjects (or in a reference sample from a reference population) is indicative of the presence of said disease, or of a risk of developing said disease.

Examples of ASCT1-associated diseases include, but are not limited to, schizophrenia, visual dysfunction, amyotrophic lateral sclerosis (ALS), microcephaly, SPATCCM (spasticity and thin corpus callosum and progressive microcephaly), cardiovascular diseases (CVD), ischemic stroke, hereditary sensory neuropathy type 1, central nervous system injury, neurological disorders, autism spectrum disorder (ASD), dementia, Alzheimer's disease, Parkinson's disease, Huntington's disease, epilepsy, diabetes including e.g. diabetic neuropathy and gestational diabetes mellitus, renal dysfunction-linked conditions including e.g. chronic renal diseases and renal cell carcinoma, vitiligo and cancers including e.g. ASCT1-associated cancers.

In some embodiments, the method of the invention is for diagnosing or for assessing a risk of developing an ASCT1-associated disease associated with a decreased ASCT1 level in a subject, and a binding of the polypeptide or variant of the invention to ASCT1 measured at step b) lower than the binding of the polypeptide or variant of the invention to ASCT1 measured in a reference population of substantially healthy subjects (or in a reference sample from a reference population) is indicative of the presence or of a risk of developing said disease.

In some embodiments, the method of the invention is for diagnosing or for assessing a risk of developing an ASCT1-associated disease associated with a decreased ASCT1 level at the cell surface in a subject, and a binding of the polypeptide or variant of the invention to ASCT1 measured at step b) lower than the binding of the polypeptide or variant of the invention to ASCT1 measured in a reference population of substantially healthy subjects (or in a reference sample from a reference population) is indicative of the presence or of a risk of developing said disease.

Examples of ASCT1-associated diseases associated with a decreased ASCT1 expression level and/or a decreased ASCT1 level at the cell surface for instance include ovarian tumors.

The present application also relates to a method for the in vivo diagnosis of a ASCT1-associated disease, comprising:
a) contacting at least one polypeptide or variant thereof according to the invention with a cell, a sample, a tissue or an organ, and
b) detecting and/or quantifying the at least one polypeptide or variant thereof according to the invention bound to ASCT1 present in the cell, sample, tissue or organ within said subject.

The present application also relates to a method for the in vivo diagnosis of ASCT1-associated disease comprising:
a) administering to a subject in need thereof at least one polypeptide or variant thereof according to the invention, and
b) detecting and/or quantifying the binding of at least one polypeptide or variant thereof according to the invention within said subject, for example by medical imaging.

In some embodiments, the polypeptide or variant according to the invention is coupled with at least one detectable label, and may be used for in vivo diagnosis by medical imaging.

In some embodiments, the polypeptide or variant administered to the subject is comprised in a diagnostic composition.

The present invention also relates to a polypeptide or variant thereof according to the invention, for use in a in vivo diagnosis method of an ASCT1-associated disease in a subject, wherein said method comprise the detection and/or measure of the level of ASCT1 using medical imaging techniques.

The present invention thus relates to an in vivo method for diagnosing a subject with or identifying a subject at risk of developing an ASCT1-associated disease comprising the steps:
a) administering to said subject a labeled polypeptide or variant thereof according to the invention; and,
b) detecting and/or measuring the binding of said labeled polypeptide or variant thereof to ASCT1 using medical imaging;
c) optionally comparing a binding measured at step b) with a reference value.

In another embodiment, the diagnosis method of the invention is an in vitro or ex vivo method, i.e., the method of the invention is performed on a cell, sample, tissue and/or organ that was obtained from a patient prior to the implementation of the method of the invention. Consequently, in some embodiments, the method of the invention does not comprise obtaining a sample from the patient, i.e., the method of the invention is non-invasive.

In some embodiments, the method of the invention is for monitoring an ASCT1-associated disease in a subject. The term “monitoring” as used herein refers to the determination of the number of cells wherein serine, alanine and/or cysteine metabolism is dysregulated in the body of a subject as a function of time, such as, for example, before, during and after a therapy against an ASCT1-associated disease.

The term “therapy against an ASCT1-associated disease” as used herein may refer to serine, alanine and/or cysteine deprivation, chemotherapy, radiation, surgery, immunotherapy, and drugs known to the skilled artisan as drugs for treating an ASCT1-associated disease.

In some embodiments, the method of monitoring of the invention comprises comparing two binding levels, such as, for example, a binding determined before treatment with a binding level determined after treatment.

In some embodiments, a decreased binding level of the polypeptide or variant of the invention after treatment is indicative of the efficacy of the treatment.

In some embodiments, a binding level after treatment equivalent or superior to the one determined before treatment is indicative of the absence of efficacy of the treatment.

The present application also relates to a method for monitoring an ASCT1-associated disease in a subject comprising the steps of:
a. contacting an effective amount of polypeptide or variant of the invention, preferably coupled with at least one contrast agent, to a cell, sample, tissue, and/or organ of said subject,
b. detecting and/or quantifying the binding of the polypeptide or variant of the invention to ASCT1 in said cell, sample, tissue, and/or organ, preferably by medical imaging,
c. treating the subject with a therapy against an ASCT1-associated disease,
d. contacting an effective amount of the polypeptide or variant of the invention, preferably coupled with at least one contrast agent to a cell, sample, tissue, and/or organ of said subject, and
e. detecting and/or quantifying the binding of the polypeptide or variant of the invention to ASCT1 in said cell, sample, tissue, and/or organ.

In some embodiments, the method of the invention further comprises a step of comparing the binding determined in step e) with the binding determined in step b), thereby monitoring an ASCT1-associated disease.

In some embodiments, the absence or the decrease of detection of ASCT1 in a cell, sample, tissue, and/or organ after a therapy against an ASCT1-associated disease, is indicative of a remission.

In other embodiments, the presence or the increase of detection of ASCT1 in a cell, sample, tissue, and/or organ after a therapy against an ASCT1-associated disease, is indicative of a remission.

The invention also relates to the polypeptide or functional variant thereof according to the invention, for use as a medicament.

The invention further relates to the nucleic acid or expression vector according to the invention, for use as a medicament.

The invention further relates to the cell according to the invention, for use as a medicament.

The invention further relates to the pharmaceutical composition according to the invention, for use as a medicament.

The present invention also relates to a method for treating a subject in need thereof, said method comprising administering to said subject the polypeptide or functional variant thereof, the nucleic acid, the expression vector, the cell or the pharmaceutical composition according to the invention.

In some embodiments, said method specifically targets ASCT1 in the subject, and not ASCT2.

The present invention also relates to the polypeptide or functional variant thereof, the nucleic acid, the expression vector, the cell or the pharmaceutical composition according to the invention, for use in the treatment of an ASCT1-associated disease.

In some embodiments, said treatment is by specifically targeting ASCT1 and not ASCT2.

The present invention also relates to a method for treating an ASCT1-associated disease in a subject in need thereof, said method comprising administering to said subject the polypeptide or functional variant thereof, the nucleic acid, the expression vector, the cell or the pharmaceutical or diagnostic composition according to the invention.

In some embodiments, said method specifically targets ASCT1 in the subject, and not ASCT2.

In some embodiments, a therapeutically effective amount of the polypeptide of the invention or variant thereof, is administered to the subject.

In some embodiments, the administration of a polypeptide or variant of the invention, modulates the flux of both the L- or D-enantiomers of the serine, alanine and/or cysteine, through the ASCT1 receptor.

The present invention also relates to the use of the polypeptide or functional variant thereof, the nucleic acid, the expression vector, the cell or the pharmaceutical composition according to the invention, for the manufacture of a medicament for the treatment of an ASCT1-associated disease.

In some embodiments, said treatment is by specifically targeting ASCT1 and not ASCT2.

The present application also relates to a method for targeting cells, samples, tissues, and/or organs expressing ASCT1, wherein said method comprises the administration of a polypeptide of the invention or variant thereof. Such method may be used, for example, for targeting therapeutic agents to cells, samples, tissues, and/or organs in a subject in need thereof. In some embodiments, the targeting method of the invention is for targeting anti-cancer drugs to cancer cells, in particular to ASCT1-expressing cancer cells.

In some embodiments, the polypeptide of the invention or variant thereof, is encapsulated with a therapeutic agent to be specifically administered to cells, samples, tissues or organs of a subject in need thereof.

In some embodiments, the polypeptide or variant thereof, the nucleic acid, the expression vector, the cell or the pharmaceutical or diagnostic composition according to the invention is to be administered at a dose determined by the skilled artisan and personally adapted to each subject.

It will be understood that the usage of the polypeptide or variant thereof, the nucleic acid, the expression vector, the cell or the pharmaceutical or diagnostic composition according to the invention will be decided by the attending physician within the scope of sound medical judgment. The specific effective amount for any particular patient will depend upon a variety of factors including the specific composition employed, the age, body weight, general health, sex and diet of the subject; the time of administration, route of administration, and like factors well known in the medical arts.

In some embodiments, a therapeutically effective amount of the polypeptide or variant thereof, the nucleic acid, the expression vector, the cell or the pharmaceutical or diagnostic composition according to the invention is administered at least once a day, twice a day, or at least three times a day.

In other embodiments, a therapeutically effective amount of the polypeptide or variant thereof, the nucleic acid, the expression vector, the cell or the pharmaceutical or diagnostic composition according to the invention is administered every two, three, four, five, or six days.

In other embodiments, a therapeutically effective amount of the polypeptide or variant thereof, the nucleic acid, the expression vector, the cell or the pharmaceutical or diagnostic composition according to the invention is administered every week, twice a week, every two weeks, or once a month.

In other embodiments, a therapeutically effective amount of the polypeptide or variant thereof, the nucleic acid, the expression vector, the cell or the pharmaceutical or diagnostic composition according to the invention is administered every month for a period at least 2, 3, 4, 5, or 6 months.

In other embodiments, a therapeutically effective amount of the polypeptide or variant thereof, the nucleic acid, the expression vector, the cell or the pharmaceutical or diagnostic composition according to the invention ranges from about 1 μg to 5 g.

In other embodiments, a therapeutically effective amount of the polypeptide or variant thereof, the nucleic acid, the expression vector, the cell or the pharmaceutical or diagnostic composition according to the invention is to be administered ranges from about 0.1 μg/kg to 1 g/kg.

In other embodiments, the polypeptide or variant thereof, the nucleic acid, the expression vector, the cell or the pharmaceutical or diagnostic composition according to the invention is to be administered in combination with another treatment for an ASCT1-associated disease.

Examples of agents for treating an ASCT1-associated disease include, but are not limited to, serine, alanine and/or cysteine deprivation, chemotherapy, radiation, surgery, protein kinases inhibitors, microtubules inhibitors, anti-metabolite agents a tumor vaccine or an immunostimulatory antibody.

In some embodiments, the method for treating an ASCT1-associated disease in a subject in need thereof, comprises administering to the subject the polypeptide or variant thereof, the nucleic acid, the expression vector, the cell or the pharmaceutical or diagnostic composition according to the invention prior to, concurrent to and/or posterior to another treatment an ASCT1-associated disease.

In some embodiments, the subject is affected, preferably is diagnosed with an ASCT1-associated disease. In other embodiments, the subject of the invention is at risk of developing an ASCT1-associated disease. Examples of risk factor for developing an ASCT1-associated disease, include, but are not limited to, genetic factors, traumatic brain injury, smoking, obesity, diabetes, alcohol, and environmental conditions.

In some embodiments, the subject of the invention is in a remission stage following an ASCT1-associated disease.

In some embodiments, the polypeptide or variant thereof, the nucleic acid, the expression vector, the cell or the pharmaceutical or diagnostic composition according to the invention is to be administered by injection, orally, topically, nasally, buccally, rectally, vaginally, intratracheally, by endoscopy, transmucosally, or by percutaneous administration.

In some embodiments, the polypeptide or variant thereof, the nucleic acid, the expression vector, the cell or the pharmaceutical or diagnostic composition according to the invention is to be administered by injection, preferably is to be systemically injected. Examples of formulations adapted to systemic injections include, but are not limited to, liquid solutions or suspensions, solid forms suitable for solution in, or suspension in, liquid prior to injection. Examples of systemic injections include, but are not limited to, intravenous, subcutaneous, intramuscular, intradermal, intravitreal, and intraperitoneal injection, or perfusion.

Preferably, when injected, the polypeptide or variant thereof, the nucleic acid, the expression vector, the cell or the pharmaceutical or diagnostic composition according to the invention is sterile. Methods for obtaining a sterile polypeptide, nucleic acid, expression vector, cell or composition, include, but are not limited to, GMP synthesis (GMP stands for “Good manufacturing practice”).

In some embodiments, the polypeptide or variant thereof, the nucleic acid, the expression vector, the cell or the pharmaceutical or diagnostic composition according to the invention are to be orally administered. Examples of formulations adapted to oral administration include, but are not limited to, solid forms, liquid forms and gels. Examples of solid forms adapted to oral administration include, but are not limited to, pill, tablet, capsule, soft gelatine capsule, hard gelatine capsule, caplet, compressed tablet, cachet, wafer, sugar-coated pill, sugar coated tablet, or dispersing/or disintegrating tablet, powder, solid forms suitable for solution in, or suspension in, liquid prior to oral administration and effervescent tablet. Examples of liquid forms adapted to oral administration include, but are not limited to, solutions, suspensions, drinkable solutions, elixirs, sealed phial, potion, drench, syrup and liquor.

In other embodiments, the polypeptide or variant thereof, the nucleic acid, the expression vector, the cell or the pharmaceutical or diagnostic composition according to the invention are to be topically administered. Examples of formulations adapted to topical administration include, but are not limited to, sticks, waxes, creams, lotions, ointments, balms, gels, masks, leave-on washes and/or the like.

Depending on the cell(s), sample(s), tissue(s) and/or organ(s) targeted, the skilled artisan can determine the technology needed for the introduction of the polypeptide or variant thereof, the nucleic acid, the expression vector, the cell or the pharmaceutical or diagnostic composition according to the invention in the targeted cell(s), sample(s), tissue(s) and/or organ(s).

In some embodiments, the polypeptide or variant thereof, the nucleic acid, the expression vector, the cell or the pharmaceutical or diagnostic composition according to the invention are to be administered in a sustained-release form. In other embodiments, the polypeptide or variant thereof, the nucleic acid, the expression vector, the cell or the pharmaceutical or diagnostic composition according to the invention comprise a delivery system that controls the release of the agent.

The present invention further relates to a method for the treatment of cancer in a subject, preferably of an ASCT1-associated cancer, more preferably of a cancer selected from the group consisting of gastrointestinal cancer, digestive tract cancer, prostate cancer, pancreas cancer, liver cancer, breast cancer, lymphoma, brain cancer, skin cancer, colon cancer, colorectal cancer, lung cancer, kidney cancer, parotid cancer, or ovarian cancer, comprising the steps of:
a) diagnosing said cancer using an in vitro or in vivo method according to the invention for diagnosing a cancer,
b) treating said cancer, preferably by chemotherapy.

In some embodiments, said cancer is an esophageal adenocarcinoma, prostate adenocarcinoma, pancreatic ductal adenocarcinoma, hepatocellular carcinoma, micropapillary carcinoma, and diffuse large B-cell lymphoma (DLBCL), neuroblastoma, liposarcoma, melanoma, colorectal adenocarcinoma, renal cell carcinoma, salivary gland carcinoma, or ovarian carcinoma.

Preferably, said cancer is an ASCT1-associated cancer.

Examples of chemotherapies include, but are not limited to:
a. alkylating agents that act mainly by forming covalent bonds between DNA bases, including, but not limited to, nitrogen mustards (e.g., cyclophosphamide), aziridines and epoxides (e.g., thiopeta), alkyl sulfonates (e.g., busulfan), nitrosureas (e.g., BCNU and CCNU), hydrazine and triazine derivatives (e.g., procarbazine and temozolomide);
b. cisplatin and its analogs that act by forming DNA adducts which lead to intra-strand and inter-strand linking leading to the formation of DNA filaments, including, but not limited to, carboplatin, cisplatin and oxaliplatin;
c. antimetabolites including but not limited to folate metabolism inhibitors (e.g., methotrexate, trimetrexate, tomudex), 5-fluoropyrimidines (e.g., 5-FU), oral fluoropyramidines (e.g., tegafur, uracil, capecitabine), nucleoside analogs (e.g., cytarabine), gemcitabine and 6-thiopurines (e.g., 6-MP and 6-TG);
d. topoisomerase-interactive agents that affect the topologic states of DNA by interfering or modulating DNA cleavage, strand passage and re-ligation, including, but not limited to, epipodophyllotoxins (e.g., etoposide and teniposide), camptothecin analogs, anthracyclines (e.g., doxorubicin, daunorubicin, epirubicin, idarubicin), mitoxantrone and losoxantrone, and dactinomycin;
e. antimicrotubule agents, which interfere with the proper polymerization/depolymerization of microtubules, including, but not limited to, vinca alkaloids (e.g., vincristine, vinorelbine and vinblastine), taxanes (e.g., paclitaxel, docetaxel) and estramustine phosphate; and
f. numerous miscellaneous agents exist which cannot be classified into any of the above groups, including but not limited to suramin, bleomycin, L-asparaginase and amifostine.

The present invention further relates to a method for the treatment of diseases characterized by a lower expression and/or function of ASCT1 in a subject, comprising the steps of:
a) diagnosing said disease using an in vitro or in vivo method according to the invention for diagnosing an ASCT1-associated disease;
b) treating said disease, preferably by serine, alanine and/or cysteine supplementation.

The present application also relates to a method for specifically inhibiting ASCT1 activity in a subject in need thereof, while not inhibiting ASCT2 activity, wherein a therapeutically effective amount of the polypeptide or variant thereof, the nucleic acid, the expression vector, the cell or the pharmaceutical according to the invention is administered to said subject.

The term “inhibiting ASCT1 activity” as used herein may refer to inhibiting the flux of serine, alanine and/or cysteine transport within a cell, or across the cell membrane, by ASCT1.

Another object of the present invention is a screening method to identify compounds modulating the level of ASCT1, said method comprising the detection and/or measure of the level of ASCT1 in a sample using the in vitro or in vivo methods of the invention.

Hence, the present invention further relates to a screening method to identify compounds modulating the level of ASCT1, using a polypeptide of the invention or variant thereof, said method comprising the steps of:
a) measuring the level of ASCT1 in a sample, preferably a biological sample from a subject, using an in vitro method of the invention;
b) contacting said sample with the tested compound;
c) measuring the level of ASCT1 in said sample using an in vitro method of the invention; and,
d) comparing the levels of ASCT1 measured at step a) and c).

The present invention also relates to a screening method to identify compounds modulating the level of ASCT1 using a polypeptide of the invention or variant thereof, said method comprising the steps of:
a) measuring the level of ASCT1 in a subject using an in vivo method of the invention;
b) contacting said subject with said compound;
c) measuring the level of ASCT1 in said subject using an in vivo method of the invention; and,
d) comparing the levels of ASCT1 measured at step a) and c).

The polypeptide or functional variant thereof according to the invention specifically recognize, bind, target and/inhibit ASCT1, while not recognizing, binding, targeting and/inhibiting ASCT2.

Therefore, the methods and uses of the invention enable specifically detecting or/and measuring the level of ASCT1, while not detecting or/and measuring the level of ASCT2.

Also, the methods and uses of the invention enable specifically diagnosing or monitoring an ASCT1-associated disease even if this disease is not also an ASCT2-associated disease.

Furthermore, the methods and uses of the invention enable treating an ASCT1-associated disease, by specifically targeting, modulating and/or inhibiting ASCT1 while not targeting, modulating and/or inhibiting ASCT2. This notably avoid or reduce possible side effects of the compounds used as a therapy.

### BRIEF DESCRIPTION OF THE DRAWINGS

FIG. 1A-D is a combination of flow cytometry plots assessing SLC overexpression in transfected cells. CHO cells were transfected with either of the following FLAG-tagged expression vectors: an empty vector (FIG. 1A), a vector containing the human SLC1A4 gene that encodes ASCT1 (FIG. 1B), a vector containing the human SLC1A5 gene that encodes ASCT2 (FIG. 1C), or a vector containing the human SLC1A1 gene that encodes EAAT3 (FIG. 1D). Forty-eight hours after transfection, cells were fixed, permeabilized, stained with an FITC-conjugated anti-FLAG antibody and analyzed by flow cytometry.

FIG. 2A-D is a combination of flow cytometry plots showing the specific binding of SNV195mFc to hASCT1/SLC1A4 and not hASCT2/SLC1A5 or hEAAT3/SLCA1. CHO cells were transfected with the FLAG-tagged expression vectors described in FIG. 1: the empty vector (FIG. 2A), or either one of the human SLC1A4 vector encoding ASCT1 (FIG. 2B), the human SLC1A5 vector encoding ASCT2 (FIG. 2C), or the human SLC1A1 vector encoding EAAT3 (FIG. 2D). SNV195mFc binding was assessed by flow cytometry using a phycoerythrin-conjugated anti-mouse IgG1 antibody.

FIG. 3A-C is a combination of flow cytometry plots assessing SLC overexpression in transfected cells. CHO cells were transfected with either of the following HA-tagged expression vectors: an empty vector (FIG. 3A), a vector containing the human SLC1A4 gene that encodes ASCT1 (FIG. 3B), or a vector containing the human SLC1A5 gene that encodes ASCT2 (FIG. 3C). Forty-eight hours after transfection, cells were fixed, permeabilized, stained with an FITC-conjugated anti-HA antibody and analyzed by flow cytometry.

FIG. 4A-L is a combination of flow cytometry plots showing the identification of the minimal sequence of SNV RBD necessary for specific and distinctive recognition of SLC1A4/ASCT1. Various SNV RBDs of different lengths were derived from the SNV retroviral envelope glycoprotein and designated by the position number of the C-ter residue, counting from the first methionine of the signal peptide. CHO cells were transfected with the HA-tagged expression vectors described in FIG. 3: the control empty vector (FIG. 4A-D), the human SLC1A4 vector encoding ASCT1 (FIG. 4E-H), or the human SLC1A5 vector encoding ASCT2 (FIG. 4I-L). Binding of SNV89mFc (FIG. 4A, 4E, 4I), SNV99mFc (FIG. 4B, 4F, 4J), SNV195mFc (FIG. 4C, 4G, 4K), or SNV200mFc (FIG. 4D, 4H, 4L) was detected using a phycoerythrin-conjugated anti-mouse IgG1 antibody and analyzed by flow cytometry.

FIG. 5A-B is a combination of histograms showing the inhibition of serine uptake and not glutamine uptake by the SNV195mFc RBD. FIG. 5A shows the quantification of the uptake of radiolabeled [³H]-serine by melanoma SK-MEL 5 cells either treated (open histograms) or not (striped histogram) with increasing amounts of SNV195mFc RBD. FIG. 5B shows the quantification of the uptake of radiolabeled [³H]-glutamine by melanoma SK-MEL 5 cells either treated (solid histogram) or not (striped histogram) with the highest amount of SNV195mFc RBD used in the serine uptake experiment. Data are expressed as means±SEM. P-values were calculated using Student's t test in GraphPad Prism 5 software (*, p≤0.05; **, p≤0.01).

FIG. 6A-B shows a combination of photographs of thin sections of a colon tumor (FIG. 6A) and normal colon tissue (FIG. 6B) labeled in parallel by immunohistochemistry with SNV195mFc. Immunohistochemistry staining appears in brown, while nuclei and cytoplasm are counterstained with hematoxylin (blue color).

FIG. 7A-B shows a combination of photographs of thin sections of a kidney tumor (FIG. 7A) and normal kidney tissue (FIG. 7B) labeled in parallel by immunohistochemistry with SNV195mFc. Immunohistochemistry staining appears in brown, while nuclei and cytoplasm are counterstained with hematoxylin (blue color).

FIG. 8A-B shows a combination of photographs of thin sections of a parotid tumor (FIG. 8A) and normal parotid tissue (FIG. 8B) labeled in parallel by immunohistochemistry with SNV195mFc. Immunohistochemistry staining appears in brown, while nuclei and cytoplasm are counterstained with hematoxylin (blue color).

FIG. 9A-B shows a combination of photographs of thin sections of an ovarian tumor (FIG. 9A) and normal ovary tissue (FIG. 9B) labeled in parallel by immunohistochemistry with SNV195mFc. Immunohistochemistry staining appears in brown, while nuclei and cytoplasm are counterstained with hematoxylin (blue color). Staining here is distinctively marked on the normal ovary tissue.

FIG. 10A-I is a combination of flow cytometry plots showing the identification of the minimal sequence of SNV RBD necessary for specific and distinctive recognition of SLC1A4/ASCT1. Various SNV RBDs of different lengths were derived from the SNV retroviral envelope glycoprotein and designated by the position number of the C-ter residue, counting from the first methionine of the signal peptide. CHO cells were transfected with the HA-tagged expression vectors described in FIG. 3: the control empty vector (FIG. 10A-C), the human SLC1A4 vector encoding ASCT1 (FIG. 10D-F), or the human SLC1A5 vector encoding ASCT2 (FIG. 10G-I). Binding of SNV94mFc (FIG. 10A, 10D, 10G), SNV97mFc (FIG. 10B, 10E, 10H), or SNV98mFc (FIG. 10C, 10F, 10I) was detected using an AlexaFluor™M647-conjugated anti-mouse IgG1 antibody and analyzed by flow cytometry.

FIG. 11A-I is a combination of flow cytometry plots showing SNV RBD capable of recognizing both SLC1A4/ASCT1 and SLC1A5/ASCT2. Various SNV RBDs of different lengths were derived from the SNV retroviral envelope glycoprotein and designated by the position number of the C-ter residue, counting from the first methionine of the signal peptide. CHO cells were transfected with the HA-tagged expression vectors described in FIG. 3: the control empty vector (FIG. 11A-C), the human SLC1A4 vector encoding ASCT1 (FIG. 11D-F), or the human SLC1A5 vector encoding ASCT2 (FIG. 11G-I). Binding of SNV210mFc (FIG. 11A, 10D, 10G), SNV220mFc (FIG. 11B, 10E, 10H), or SNV258mFc (FIG. 11C, 10F, 10I) was detected using a phycoerythrin-conjugated anti-mouse IgG1 antibody and analyzed by flow cytometry.

TABLE OF SEQUENCES

| SEQ | | |
|---|---|---|
| ID | Sequence | |
| NO | function | Sequence |
| 1 | Polypeptide of | (X)ₙYPLQQLWGLPCDCSGGYVSSIPTYYTYSLDCGGSTAYLTYGSGT |
| | formula A | GSWSWGGGFKQQWEC(Y)ₘ |
| | | (X)ₙ is a sequence of n amino acids; |
| | | (Y)ₘ is a sequence of m amino acids; |
| | | n ≥ 0 and m ≥ 0 |
| 2 | SNV95 | YPLQQLWGLPCDCSGGYVSSIPTYYTYSLDCGGSTAYLTYGSGTGS |
| | | WSWGGGFKQQWEC |
| 3 | SNV200-258 | RERLEEIIRHSYPSVQYHPLALPRSRGVDLDPQTSDILEATHQVLNAT |
| | | NPKLAENCWLC |
| 4 | SNV96 | YPLQQLWGLPCDCSGGYVSSIPTYYTYSLDCGGSTAYLTYGSGTGS |
| | | WSWGGGFKQQWECV |
| 5 | SNV97 | YPLQQLWGLPCDCSGGYVSSIPTYYTYSLDCGGSTAYLTYGSGTGS |
| | | WSWGGGFKQQWECVF |
| 6 | SNV98 | YPLQQLWGLPCDCSGGYVSSIPTYYTYSLDCGGSTAYLTYGSGTGS |
| | | WSWGGGFKQQWECVFK |
| 7 | SNV99 | YPLQQLWGLPCDCSGGYVSSIPTYYTYSLDCGGSTAYLTYGSGTGS |
| | | WSWGGGFKQQWECVFKP |
| 8 | SNV96-199 | VFKPKIIPSVQGQPGPCPSECLQIATQMHSTCYEKTQECTLLGKTYFT |
| | | AILQKTKLGSYEDGPNKLIQASCTGTVGKPVCWDPVAPVYVSDGGG |
| | | PTDMIREESV |
| 9 | SNV97-199 | FKPKIIPSVQGQPGPCPSECLQIATQMHSTCYEKTQECTLLGKTYFTAI |
| | | LQKTKLGSYEDGPNKLIQASCTGTVGKPVCWDPVAPVYVSDGGGPT |
| | | DMIREESV |
| 10 | SNV98-199 | KPKIIPSVQGQPGPCPSECLQIATQMHSTCYEKTQECTLLGKTYFTAI |
| | | LQKTKLGSYEDGPNKLIQASCTGTVGKPVCWDPVAPVYVSDGGGPT |
| | | DMIREESV |
| 11 | SNV99-199 | PKIIPSVQGQPGPCPSECLQIATQMHSTCYEKTQECTLLGKTYFTAIL |
| | | QKTKLGSYEDGPNKLIQASCTGTVGKPVCWDPVAPVYVSDGGGPT |
| | | DMIREESV |
| 12 | SNV100-199 | KIIPSVQGQPGPCPSECLQIATQMHSTCYEKTQECTLLGKTYFTAILQ |
| | | KTKLGSYEDGPNKLIQASCTGTVGKPVCWDPVAPVYVSDGGGPTD |
| | | MIREESV |
| 13 | SNV258 | MDCLTNLRSAEGKVDQASKILILLVAWWGFGTTAEGYPLQQLWGL |
| | | PCDCSGGYVSSIPTYYTYSLDCGGSTAYLTYGSGTGSWSWGGGFKQ |
| | | QWECVFKPKIIPSVQGQPGPCPSECLQIATQMHSTCYEKTQECTLLG |
| | | KTYFTAILQKTKLGSYEDGPNKLIQASCTGTVGKPVCWDPVAPVYV |
| | | SDGGGPTDMIREESVRERLEEIIRHSYPSVQYHPLALPRSRGVDLDPQ |
| | | TSDILEATHQVLNATNPKLAENCWLC |
| 14 | SNV95 | TACCCCTTGCAGCAACTTTGGGGACTGCCTTGTGACTGTTCCGGG |
| | nucleic acid | GGATATGTCTCCTCCATACCTACCTATTACACCTACTCCCTCGATT |
| | | GTGGTGGCTCCACCGCCTACCTGACTTACGGATCTGGTACAGGGA |
| | | GTTGGAGCTGGGGAGGGGGATTTAAACAACAGTGGGAATGT |
| 15 | SNV96 | TACCCCTTGCAGCAACTTTGGGGACTGCCTTGTGACTGTTCCGGG |
| | nucleic acid | GGATATGTCTCCTCCATACCTACCTATTACACCTACTCCCTCGATT |
| | | GTGGTGGCTCCACCGCCTACCTGACTTACGGATCTGGTACAGGGA |
| | | GTTGGAGCTGGGGAGGGGGATTTAAACAACAGTGGGAATGTGTG |
| 16 | SNV97 | TACCCCTTGCAGCAACTTTGGGGACTGCCTTGTGACTGTTCCGGG |
| | nucleic acid | GGATATGTCTCCTCCATACCTACCTATTACACCTACTCCCTCGATT |
| | | GTGGTGGCTCCACCGCCTACCTGACTTACGGATCTGGTACAGGGA |
| | | GTTGGAGCTGGGGAGGGGGATTTAAACAACAGTGGGAATGTGTG |
| | | TTT |
| 17 | SNV98 | TACCCCTTGCAGCAACTTTGGGGACTGCCTTGTGACTGTTCCGGG |
| | nucleic acid | GGATATGTCTCCTCCATACCTACCTATTACACCTACTCCCTCGATT |
| | | GTGGTGGCTCCACCGCCTACCTGACTTACGGATCTGGTACAGGGA |
| | | GTTGGAGCTGGGGAGGGGGATTTAAACAACAGTGGGAATGTGTG |
| | | TTTAAA |
| 18 | SNV99 | TACCCCTTGCAGCAACTTTGGGGACTGCCTTGTGACTGTTCCGGG |
| | nucleic acid | GGATATGTCTCCTCCATACCTACCTATTACACCTACTCCCTCGATT |
| | | GTGGTGGCTCCACCGCCTACCTGACTTACGGATCTGGTACAGGGA |
| | | GTTGGAGCTGGGGAGGGGGATTTAAACAACAGTGGGAATGTGTG |
| | | TTTAAACCT |
| 19 | SNV258 | TACCCCTTGCAGCAACTTTGGGGACTGCCTTGTGACTGTTCCGGG |
| | nucleic acid | GGATATGTCTCCTCCATACCTACCTATTACACCTACTCCCTCGATT |
| | | GTGGTGGCTCCACCGCCTACCTGACTTACGGATCTGGTACAGGGA |
| | | GTTGGAGCTGGGGAGGGGGATTTAAACAACAGTGGGAATGTGTG |
| | | TTTAAACCTAAGATCATACCCTCTGTGCAGGGGCAGCCAGGGCCC |
| | | TGCCCATCTGAATGCCTCCAGATAGCTACTCAAATGCATTCCACT |
| | | TGTTATGAAAAGACTCAGGAATGCACCCTCCTGGGAAAAACCTA |
| | | TTTTACTGCCATCCTACAGAAAACAAAGCTAGGCTCGTATGAAGA |
| | | CGGGCCTAATAAACTGATCCAGGCCTCTTGCACGGGAACCGTAG |
| | | GGAAACCAGTATGTTGGGACCCTGTAGCCCCTGTGTATGTCTCTG |
| | | ATGGCGGCGGTCCCACTGACATGATTCGGGAAGAGTCTGTGCGT |
| | | GAAAGACTAGAGGAAATCATCAGGCACAGCTACCCCTCCGTACA |
| | | GTATCACCCTTTAGCCCTGCCCCGATCAAGAGGAGTAGATCTGGA |
| | | TCCCCAGACGTCTGACATACTGGAAGCTACTCACCAGGTCCTTAA |
| | | TGCCACTAATCCCAAGCTAGCAGAGAACTGCTGGCTTTGT |
| 20 | Human | MEKSNETNGYLDSAQAGPAAGPGAPGTAAGRARRCAGFLRRQALV |
| | SLC1A4/ | LLTVSGVLAGAGLGAALRGLSLSRTQVTYLAFPGEMLLRMLRMIILP |
| | ASCT1 | LVVCSLVSGAASLDASCLGRLGGIAVAYFGLTTLSASALAVALAFIIK |
| | | PGSGAQTLQSSDLGLEDSGPPPVPKETVDSFLDLARNLFPSNLVVAA |
| | | FRTYATDYKVVTQNSSSGNVTHEKIPIGTEIEGMNILGLVLFALVLGV |
| | | ALKKLGSEGEDLIRFFNSLNEATMVLVSWIMWYVPVGIMFLVGSKI |
| | | VEMKDIIVLVTSLGKYIFASILGHVIHGGIVLPLIYFVFTRKNPFRFLL |
| | | GLLAPFATAFATCSSSATLPSMMKCIEENNGVDKRISRFILPIGATVN |
| | | MDGAAIFQCVAAVFIAQLNNVELNAGQIFTILVTATASSVGAAGVPA |
| | | GGVLTIAIILEAIGLPTHDLPLILAVDWIVDRTTTVVNVEGDALGAGI |
| | | LHHLNQKATKKGEQELAEVKVEAIPNCKSEEETSPLVTHQNPAGPV |
| | | ASAPELESKESVL |
| 21 | Human | CCCGCACTCTGCGCCTCTCCTCGCCTTTCTCGCACCTGCTCCTGCG |
| | SLC1A4/ | CCAGGCCCGGAGACCCCCGGGGCGGCTTCCCAGAACCTGCGGAG |
| | ASCT1 | CACAACTGGCCGACCGACCCATTCATTGGGAACCCGTCTTTTGCC |
| | nucleic acid | AGAGCCCACGTCCCCTGCCACCTCTAGCTCGGAGCGGCGTGTAGC |
| | | GCCATGGAGAAGAGCAACGAGACCAACGGCTACCTTGACAGCGC |
| | | TCAGGCGGGGCCTGCGGCCGGGCCCGGAGCTCCGGGGACCGCGG |
| | | CGGGACGCGCACGGCGTTGCGCGCGCTTCCTGCGGCGCCAAGCG |
| | | CTGGTGCTGCTCACCGTGTCCGGGGTGCTGGCGGGCGCGGGCCTG |
| | | GGCGCGGCGTTGCGCGGGCTCAGCCTGAGCCGCACGCAGGTCAC |
| | | CTACCTGGCCTTCCCCGGCGAGATGCTGCTCCGCATGCTGCGCAT |
| | | GATCATCCTGCCGCTGGTGGTCTGCAGCCTGGTGTCGGGCGCCGC |
| | | CTCGCTCGATGCCAGCTGCCTCGGGCGTCTGGGCGGCATCCGTGT |
| | | CGCCTACTTTGGCCTCACCACACTGAGTGCCTCGGCGCTCGCCGT |
| | | GGCCTTGGCGTTCATCATCAAGCCAGGATCCGGTGCGCAGACCCT |
| | | TCAGTCCAGCGACCTGGGGCTGGAGGACTCGGGGCCTCCTCCTGT |
| | | CCCCAAAGAGACGGTGGACTCTTTCCTCGACCTGGCCAGAAACCT |
| | | GTTTCCCTCCAATCTTGTGGTTGCAGCTTTCCGTACGTATGCAACC |
| | | GATTATAAAGTCGTGACCCAGAACAGCAGCTCTGGAAATGTAAC |
| | | CCATGAAAAGATCCCCATAGGCACTGAGATAGAAGGGATGAACA |
| | | TTTTAGGATTGGTCCTGTTTGCTCTGGTGTTAGGAGTGGCCTTAAA |
| | | GAAACTAGGCTCCGAAGGAGAAGACCTCATCCGTTTCTTCAATTC |
| | | CCTCAACGAGGCGACGATGGTGCTGGTGTCCTGGATTATGTGGTA |
| | | CGTACCTGTGGGCATCATGTTCCTTGTTGGAAGCAAGATCGTGGA |
| | | AATGAAAGACATCATCGTGCTGGTGACCAGCCTGGGGAAATACA |
| | | TCTTCGCATCTATATTGGGCCATGTTATTCATGGAGGAATTGTTCT |
| | | GCCACTTATTTATTTTGTTTTCACACGAAAAAACCCATTCAGATTC |
| | | CTCCTGGGCCTCCTCGCCCCATTTGCGACAGCATTTGCTACCTGCT |
| | | CCAGCTCAGCGACCCTTCCCTCTATGATGAAGTGCATTGAAGAGA |
| | | ACAATGGTGTGGACAAGAGGATCAGCAGGTTTATTCTCCCCATCG |
| | | GGGCCACCGTGAACATGGACGGAGCAGCCATCTTCCAGTGTGTG |
| | | GCCGCGGTGTTCATTGCGCAACTCAACAACATAGAGCTCAACGC |
| | | AGGACAGATTTTCACCATTCTAGTGACTGCCACAGCGTCCAGTGT |
| | | TGGAGCAGCAGGCGTGCCAGCTGGAGGGGTCCTCACCATTGCCA |
| | | TTATCCTGGAGGCCATTGGGCTGCCTACTCATGACCTGCCTCTGA |
| | | TCCTGGCTGTGGACTGGATTGTGGACCGGACCACCACGGTGGTGA |
| | | ATGTGGAAGGGGATGCCCTGGGTGCAGGCATTCTCCACCACCTG |
| | | AATCAGAAGGCAACAAAGAAAGGCGAGCAGGAACTTGCTGAGG |
| | | TGAAAGTGGAAGCCATCCCCAACTGCAAGTCTGAGGAGGAGACA |
| | | TCGCCCCTGGTGACACACCAGAACCCCGCTGGCCCCGTGGCCAGT |
| | | GCCCCAGAACTGGAATCCAAGGAGTCGGTTCTGTGATGGGGCTG |
| | | GGCTTTGGGCTTGCCTGCCAGCAGTGATGTCCCACCCTGTTCACC |
| | | CAGCCGCCAGTCATGGACACAGGGCACTGCCTTGCCAACTTTTAC |
| | | CCTCCCAAGCAATGCTTTGGCCCAGTCGCTGGCCTGAGGCTTACC |
| | | TCTCGGCACTGGCATTGGGCTCCCCAGCCGGAACTGGTTACCAAG |
| | | GACAAGGACACTCTGACATTCGGCTTGATCCATGTCCAGGTGCAA |
| | | CTGTGTGTACACCAGGGATCTGTTTGGAAACAACCCCTTGAGCTG |
| | | CCAGGCTCAAGAAATCATGGACTCACAGGGTCCTGTGTGGTT |
| 22 | Human | MVADPPRDSKGLAAAEPTANGGLALASIEDQGAAAGGYCGSRDQV |
| | SLC1A5/ | RRCLRANLLVLLTVVAVVAGVALGLGVSGAGGALALGPERLSAFVF |
| | ASCT2 | PGELLLRLLRMIILPLVVCSLIGGAASLDPGALGRLGAWALLFFLVTT |
| | | LLASALGVGLALALQPGAASAAINASVGAAGSAENAPSKEVLDSFL |
| | | DLARNIFPSNLVSAAFRSYSTTYEERNITGTRVKVPVGQEVEGMNIL |
| | | GLVVFAIVFGVALRKLGPEGELLIRFFNSFNEATMVLVSWIMWYAP |
| | | VGIMFLVAGKIVEMEDVGLLFARLGKYILCCLLGHAIHGLLVLPLIYF |
| | | LFTRKNPYRFLWGIVTPLATAFGTSSSSATLPLMMKCVEENNGVAK |
| | | HISRFILPIGATVNMDGAALFQCVAAVFIAQLSQQSLDFVKIITILVTA |
| | | TASSVGAAGIPAGGVLTLAIILEAVNLPVDHISLILAVDWLVDRSCTV |
| | | LNVEGDALGAGLLQNYVDRTESRSTEPELIQVKSELPLDPLPVPTEE |
| | | GNPLLKHYRGPAGDATVASEKESVM |
| 23 | Human | ATGGTGGCCGATCCTCCTCGAGACTCCAAGGGGCTCGCAGCGGC |
| | SLC1A5/ | GGAGCCCACCGCCAACGGGGGCCTGGCGCTGGCCTCCATCGAGG |
| | ASCT2 | ACCAAGGCGCGGCAGCAGGCGGCTACTGCGGTTCCCGGGACCAG |
| | nucleic acid | GTGCGCCGCTGCCTTCGAGCCAACCTGCTTGTGCTGCTGACAGTG |
| | | GTGGCCGTGGTGGCCGGCGTGGCGCTGGGACTGGGGGTGTCGGG |
| | | GGCCGGGGGTGCGCTGGCGTTGGGCCCGGAGCGCTTGAGCGCCT |
| | | TCGTCTTCCCGGGCGAGCTGCTGCTGCGTCTGCTGCGGATGATCA |
| | | TCTTGCCGCTGGTGGTGTGCAGCTTGATCGGCGGCGCCGCCAGCC |
| | | TGGACCCCGGCGCGCTCGGCCGTCTGGGCGCCTGGGCGCTGCTCT |
| | | TTTTCCTGGTCACCACGCTGCTGGCGTCGGCGCTCGGAGTGGGCT |
| | | TGGCGCTGGCTCTGCAGCCGGGCGCCGCCTCCGCCGCCATCAACG |
| | | CCTCCGTGGGAGCCGCGGGCAGTGCCGAAAATGCCCCCAGCAAG |
| | | GAGGTGCTCGATTCGTTCCTGGATCTTGCGAGAAATATCTTCCCT |
| | | TCCAACCTGGTGTCAGCAGCCTTTCGCTCATACTCTACCACCTAT |
| | | GAAGAGAGGAATATCACCGGAACCAGGGTGAAGGTGCCCGTGGG |
| | | GCAGGAGGTGGAGGGGATGAACATCCTGGGCTTGGTAGTGTTTG |
| | | CCATCGTCTTTGGTGTGGCGCTGCGGAAGCTGGGGCCTGAAGGG |
| | | GAGCTGCTTATCCGCTTCTTCAACTCCTTCAATGAGGCCACCATG |
| | | GTTCTGGTCTCCTGGATCATGTGGTATGCCCCTGTGGGCATCATG |
| | | TTCCTGGTGGCTGGCAAGATCGTGGAGATGGAGGATGTGGGTTTA |
| | | CTCTTTGCCCGCCTTGGCAAGTACATTCTGTGCTGCCTGCTGGGTC |
| | | ACGCCATCCATGGGCTCCTGGTACTGCCCCTCATCTACTTCCTCTT |
| | | CACCCGCAAAAACCCCTACCGCTTCCTGTGGGGCATCGTGACGCC |
| | | GCTGGCCACTGCCTTTGGGACCTCTTCCAGTTCCGCCACGCTGCC |
| | | GCTGATGATGAAGTGCGTGGAGGAGAATAATGGCGTGGCCAAGC |
| | | ACATCAGCCGTTTCATCCTGCCCATCGGCGCCACCGTCAACATGG |
| | | ACGGTGCCGCGCTCTTCCAGTGCGTGGCCGCAGTGTTCATTGCAC |
| | | AGCTCAGCCAGCAGTCCTTGGACTTCGTAAAGATCATCACCATCC |
| | | TGGTCACGGCCACAGCGTCCAGCGTGGGGGCAGCGGGCATCCCT |
| | | GCTGGAGGTGTCCTCACTCTGGCCATCATCCTCGAAGCAGTCAAC |
| | | CTCCCGGTCGACCATATCTCCTTGATCCTGGCTGTGGACTGGCTA |
| | | GTCGACCGGTCCTGTACCGTCCTCAATGTAGAAGGTGACGCTCTG |
| | | GGGGCAGGACTCCTCCAAAATTACGTGGACCGTACGGAGTCGAG |
| | | AAGCACAGAGCCTGAGTTGATACAAGTGAAGAGTGAGCTGCCCC |
| | | TGGATCCGCTGCCAGTCCCCACTGAGGAAGGAAACCCCCTCCTCA |
| | | AACACTATCGGGGGCCCGCAGGGGATGCCACGGTCGCCTCTGAG |
| | | AAGGAATCAGTCATGTAA |
| 24 | Rabbit Fc | APSTCSKPTCPPPELLGGPSVFIFPPKPKDTLMISRTPEVTCVVVDVSQ |
| | | DDPEVQFTWYINNEQVRTARPPLREQQFDCTIRVVSTLPIAHQDWLR |
| | | GKEFKCKVHNKALPAPIEKTISKARGQPLEPKVYTMGPPREELSSRS |
| | | VSLTCMINGFYPSDISVEWEKNGKAEDNYKTTPAVLDSDGSYFLYS |
| | | KLSVPTSEWQRGDVFTCSVMHEALHNHYTQKSISRSPGK |
| 25 | Rabbit Fc | GCACCCTCGACATGCAGCAAGCCCACGTGCCCACCCCCTGAACTC |
| | nucleic acid | CTGGGGGGACCGTCTGTCTTCATCTTCCCCCCAAAACCCAAGGAC |
| | | ACCCTCATGATCTCACGCACCCCCGAGGTCACATGCGTGGTGGTG |
| | | GACGTGAGCCAGGATGACCCCGAGGTGCAGTTCACATGGTACAT |
| | | AAACAACGAGCAGGTGCGCACCGCCCGGCCGCCGCTACGGGAGC |
| | | AGCAGTTCAACAGCACGATCCGCGTGGTCAGCACCCTCCCCATCA |
| | | CGCACCAGGACTGGCTGAGGGGCAAGGAGTTCAAGTGCAAAGTC |
| | | CACAACAAGGCACTCCCGGCCCCCATCGAGAAAACCATCTCCAA |
| | | AGCCAGAGGGCAGCCCCTGGAGCCGAAGGTCTACACCATGGGCC |
| | | CTCCCCGGGAGGAGCTGAGCAGCAGGTCGGTCAGCCTGACCTGC |
| | | ATGATCAACGGCTTCTACCCTTCCGACATCTCGGTGGAGTGGGAG |
| | | AAGAACGGGAAGGCAGAGGACAACTACAAGACCACGCCGGCCG |
| | | TGCTGGACAGCGACGGCTCCTACTTCCTCTACAACAAGCTCTCAG |
| | | TGCCCACGAGTGAGTGGCAGCGGGGCGACGTCTTCACCTGCTCCG |
| | | TGATGCACGAGGCCTTGCACAACCACTACACGCAGAAGTCCATCT |
| | | CCCGCTCTCCGGGTAAATGA |
| 26 | Mouse Fc | VDVPRDCGCKPCICTVPEVSSVFIFPPKPKDVLTITLTPKVTCVVVDIS |
| | | KDDPEVQFSWFVDDVEVHTAQTQPREEQFNSTFRSVSELPIMHQDW |
| | | LNGKEFKCRVNSAAFPAPIEKTISKTKGRPKAPQVYTIPPPKEQMAK |
| | | DKVSLTCMITDFFPEDITVEWQWNGQPAENYKNTQPIMDTDGSYFV |
| | | YSKLNVQKSNWEAGNTFTCSVLHEGLHNHHTEKSLSHSPGK |
| 27 | Mouse Fc | GTCGACGTGCCCAGGGATTGTGGTTGTAAGCCTTGCATATGTACA |
| | nucleic acid | GTCCCAGAAGTATCATCTGTCTTCATCTTCCCCCCAAAGCCCAAG |
| | | GATGTGCTCACCATTACTCTGACTCCTAAGGTCACGTGTGTTGTG |
| | | GTAGACATCAGCAAGGATGATCCCGAGGTCCAGTTCAGCTGGTTT |
| | | GTAGATGATGTGGAGGTGCACACAGCTCAGACGCAACCCCGGGA |
| | | GGAGCAGTTCAACAGCACTTTCCGCTCAGTCAGTGAACTTCCCAT |
| | | CATGCACCAGGACTGGCTCAATGGCAAGGAGTTCAAATGCAGGG |
| | | TCAACAGTGCAGCTTTCCCTGCCCCCATCGAGAAAACCATCTCCA |
| | | AAACCAAAGGCAGACCGAAGGCTCCACAGGTGTACACCATTCCA |
| | | CCTCCCAAGGAGCAGATGGCCAAGGATAAAGTCAGTCTGACCTG |
| | | CATGATAACAGACTTCTTCCCTGAAGACATTACTGTGGAGTGGCA |
| | | GTGGAATGGGCAGCCAGCGGAGAACTACAAGAACACTCAGCCCA |
| | | TCATGGACACAGATGGCTCTTACTTCGTCTACAGCAAGCTCAATG |
| | | TGCAGAAGAGCAACTGGGAGGCAGGAAATACTTTCACCTGCTCT |
| | | GTGTTACATGAGGGCCTGCACAACCACCATACTGAGAAGAGCCT |
| | | CTCCCACTCTCCTGGTAAATGA |
| 28 | Human IL-2 | MYRMQLLSCIALSLALVTNS |
| | signal peptide | |
| 29 | Human | MKWVTFISLLFLFSSAYS |
| | albumin signal | |
| | peptide | |
| 30 | Human | MAFLWLLSCWALLGTTFG |
| | chymotrypsino | |
| | gen signal | |
| | peptide | |
| 31 | Human | MNLLLILTFVAAAVA |
| | trypsinogen-2 | |
| | signal peptide | |
| 32 | Gaussia | MGVKVLFALICIAVAEA |
| | luciferase | |
| | signal peptide | |
| 33 | Mouse IgM | MKFSWVMFFLMAVVTGVNSEF |
| | signal peptide | |
| 34 | SNV200mFc | MDCLTNLRSAEGKVDQASKILILLVAWWGFGTTAEGYPLQQLWGL |
| | | PCDCSGGYVSSIPTYYTYSLDCGGSTAYLTYGSGTGSWSWGGGFKQ |
| | | QWECVFKPKIIPSVQGQPGPCPSECLQIATQMHSTCYEKTQECTLLG |
| | | KTYFTAILQKTKLGSYEDGPNKLIQASCTGTVGKPVCWDPVAPVYV |
| | | 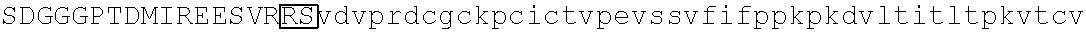 |
| | | vvdiskddpevqfswfvddvevhtaqtqpreeqfnstfrsvselpimhqdwlngkefkcrvnsaafpa |
| | | piektisktkgrpkapqvytipppkeqmakdkvsltcmitdffpeditvewqwngqpaenykntqpi |
| | | mdtdgsyfvysklnvqksnweagntftcsvlheglhnhhtekslshspgk |
| 35 | SNV195mFc | MDCLTNLRSAEGKVDQASKILILLVAWWGFGTTAEGYPLQQLWGL |
| | | PCDCSGGYVSSIPTYYTYSLDCGGSTAYLTYGSGTGSWSWGGGFKQ |
| | | QWECVFKPKIIPSVQGQPGPCPSECLQIATQMHSTCYEKTQECTLLG |
| | | KTYFTAILQKTKLGSYEDGPNKLIQASCTGTVGKPVCWDPVAPVYV |
| | | 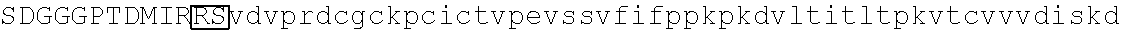 |
| | | dpevqfswfvddvevhtaqtqpreeqfnstfrsvselpimhqdwlngkefkcrvnsaafpapiektiskt |
| | | kgrpkapqvytipppkeqmakdkvsltcmitdffpeditvewqwngqpaenykntqpimdtdgsyf |
| | | vysklnvqksnweagntftcsvlheglhnhhtekslshspgk |
| 36 | SNV99mFc | MDCLTNLRSAEGKVDQASKILILLVAWWGFGTTAEGYPLQQLWGL |
| | | PCDCSGGYVSSIPTYYTYSLDCGGSTAYLTYGSGTGSWSWGGGFKQ |
| | | 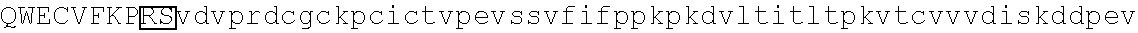 |
| | | qfswfvddvevhtaqtqpreeqfnstfrsvselpimhqdwlngkefkcrvnsaafpapiektisktkgrp |
| | | kapqvytipppkeqmakdkvsltcmitdffpeditvewqwngqpaenykntqpimdtdgsyfvysk |
| | | Invqksnweagntftcsvlheglhnhhtekslshspgk |
| 37 | SNV89mFc | MDCLTNLRSAEGKVDQASKILILLVAWWGFGTTAEGYPLQQLWGL |
| | | 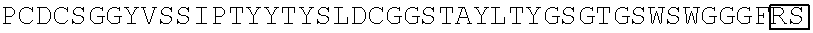 |
| | | vdvprdcgckpcictvpevssvfifppkpkdvltitltpkvtcvvvdiskddpevqfswfvddvevhtaq |
| | | tqpreeqfnstfrsvselpimhqdwlngkefkcrvnsaafpapiektisktkgrpkapqvytipppkeq |
| | | makdkvsltcmitdffpeditvewqwngqpaenykntqpimdtdgsyfvysklnvqksnweagntft |
| | | csvlheglhnhhtekslshspgk |
| 38 | SNV200mFc | ATGGACTGTCTCACCAACCTCCGATCCGCTGAGGGTAAAGTTGACC |
| | nucleic acid | AGGCGAGCAAAATCCTAATTCTCCTTGTGGCTTGGTGGGGGTTTGG |
| | | GACCACTGCCGAAGGTTACCCCTTGCAGCAACTTTGGGGACTGCCT |
| | | TGTGACTGTTCCGGGGGATATGTCTCCTCCATACCTACCTATTACAC |
| | | CTACTCCCTCGATTGTGGTGGCTCCACCGCCTACCTGACTTACGGAT |
| | | CTGGTACAGGGAGTTGGAGCTGGGGAGGGGGATTTAAACAACAGT |
| | | GGGAATGTGTGTTTAAACCTAAGATCATACCCTCTGTGCAGGGGCA |
| | | GCCAGGGCCCTGCCCATCTGAATGCCTCCAGATAGCTACTCAAATG |
| | | CATTCCACTTGTTATGAAAAGACTCAGGAATGCACCCTCCTGGGAA |
| | | AAACCTATTTTACTGCCATCCTACAGAAAACAAAGCTAGGCTCGTA |
| | | TGAAGACGGGCCTAATAAACTGATCCAGGCCTCTTGCACGGGAACC |
| | | GTAGGGAAACCAGTATGTTGGGACCCTGTAGCCCCTGTGTATGTCT |
| | | CTGATGGCGGCGGTCCCACTGACATGATTCGGGAAGAGTCTGTGCG |
| | | 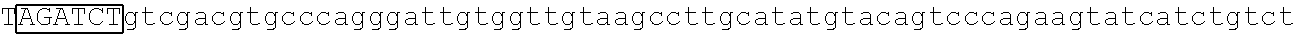 |
| | | tcatcttccccccaaagcccaaggatgtgctcaccattactctgactcctaaggtcacgtgtgttgtggtagacatca |
| | | gcaaggatgatcccgaggtccagttcagctggtttgtagatgatgtggaggtgcacacagctcagacgcaacccc |
| | | gggaggagcagttcaacagcactttccgctcagtcagtgaacttcccatcatgcaccaggactggctcaatggcaa |
| | | ggagttcaaatgcagggtcaacagtgcagctttccctgcccccatcgagaaaaccatctccaaaaccaaaggcag |
| | | accgaaggctccacaggtgtacaccattccacctcccaaggagcagatggccaaggataaagtcagtctgacctg |
| | | catgataacagacttcttccctgaagacattactgtggagtggcagtggaatgggcagccagcggagaactacaa |
| | | gaacactcagcccatcatggacacagatggctcttacttcgtctacagcaagctcaatgtgcagaagagcaactgg |
| | | gaggcaggaaatactttcacctgctctgtgttacatgagggcctgcacaaccaccatactgagaagagcctctccc |
| | | actctcctggtaaatga |
| 39 | SNV195mFc | ATGGACTGTCTCACCAACCTCCGATCCGCTGAGGGTAAAGTTGACC |
| | nucleic acid | AGGCGAGCAAAATCCTAATTCTCCTTGTGGCTTGGTGGGGGTTTGG |
| | | GACCACTGCCGAAGGTTACCCCTTGCAGCAACTTTGGGGACTGCCT |
| | | TGTGACTGTTCCGGGGGATATGTCTCCTCCATACCTACCTATTACAC |
| | | CTACTCCCTCGATTGTGGTGGCTCCACCGCCTACCTGACTTACGGAT |
| | | CTGGTACAGGGAGTTGGAGCTGGGGAGGGGGATTTAAACAACAGT |
| | | GGGAATGTGTGTTTAAACCTAAGATCATACCCTCTGTGCAGGGGCA |
| | | GCCAGGGCCCTGCCCATCTGAATGCCTCCAGATAGCTACTCAAATG |
| | | CATTCCACTTGTTATGAAAAGACTCAGGAATGCACCCTCCTGGGAA |
| | | AAACCTATTTTACTGCCATCCTACAGAAAACAAAGCTAGGCTCGTA |
| | | TGAAGACGGGCCTAATAAACTGATCCAGGCCTCTTGCACGGGAACC |
| | | GTAGGGAAACCAGTATGTTGGGACCCTGTAGCCCCTGTGTATGTCT |
| | | 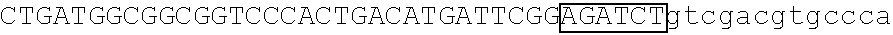 |
| | | gggattgtggttgtaagccttgcatatgtacagtcccagaagtatcatctgtcttcatcttccccccaaagcccaagga |
| | | tgtgctcaccattactctgactcctaaggtcacgtgtgttgtggtagacatcagcaaggatgatcccgaggtccagtt |
| | | cagctggtttgtagatgatgtggaggtgcacacagctcagacgcaaccccgggaggagcagttcaacagcacttt |
| | | ccgctcagtcagtgaacttcccatcatgcaccaggactggctcaatggcaaggagttcaaatgcagggtcaacagt |
| | | gcagctttccctgcccccatcgagaaaaccatctccaaaaccaaaggcagaccgaaggctccacaggtgtacac |
| | | cattccacctcccaaggagcagatggccaaggataaagtcagtctgacctgcatgataacagacttcttccctgaa |
| | | gacattactgtggagtggcagtggaatgggcagccagcggagaactacaagaacactcagcccatcatggacac |
| | | agatggctcttacttcgtctacagcaagctcaatgtgcagaagagcaactgggaggcaggaaatactttcacctgct |
| | | ctgtgttacatgagggcctgcacaaccaccatactgagaagagcctctcccactctcctggtaaatga |
| 40 | SNV99mFc | ATGGACTGTCTCACCAACCTCCGATCCGCTGAGGGTAAAGTTGACC |
| | nucleic acid | AGGCGAGCAAAATCCTAATTCTCCTTGTGGCTTGGTGGGGGTTTGG |
| | | GACCACTGCCGAAGGTTACCCCTTGCAGCAACTTTGGGGACTGCCT |
| | | TGTGACTGTTCCGGGGGATATGTCTCCTCCATACCTACCTATTACAC |
| | | CTACTCCCTCGATTGTGGTGGCTCCACCGCCTACCTGACTTACGGAT |
| | | CTGGTACAGGGAGTTGGAGCTGGGGAGGGGGATTTAAACAACAGT |
| | | 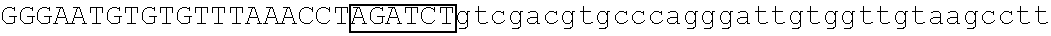 |
| | | gcatatgtacagtcccagaagtatcatctgtcttcatcttccccccaaagcccaaggatgtgctcaccattactctgac |
| | | tcctaaggtcacgtgtgttgtggtagacatcagcaaggatgatcccgaggtccagttcagctggtttgtagatgatgt |
| | | ggaggtgcacacagctcagacgcaaccccgggaggagcagttcaacagcactttccgctcagtcagtgaacttc |
| | | ccatcatgcaccaggactggctcaatggcaaggagttcaaatgcagggtcaacagtgcagctttccctgcccccat |
| | | cgagaaaaccatctccaaaaccaaaggcagaccgaaggctccacaggtgtacaccattccacctcccaaggag |
| | | cagatggccaaggataaagtcagtctgacctgcatgataacagacttcttccctgaagacattactgtggagtggca |
| | | gtggaatgggcagccagcggagaactacaagaacactcagcccatcatggacacagatggctcttacttcgtcta |
| | | cagcaagctcaatgtgcagaagagcaactgggaggcaggaaatactttcacctgctctgtgttacatgagggcctg |
| | | cacaaccaccatactgagaagagcctctcccactctcctggtaaatga |
| 41 | SNV89mFc | ATGGACTGTCTCACCAACCTCCGATCCGCTGAGGGTAAAGTTGACC |
| | nucleic acid | AGGCGAGCAAAATCCTAATTCTCCTTGTGGCTTGGTGGGGGTTTGG |
| | | GACCACTGCCGAAGGTTACCCCTTGCAGCAACTTTGGGGACTGCCT |
| | | TGTGACTGTTCCGGGGGATATGTCTCCTCCATACCTACCTATTACAC |
| | | CTACTCCCTCGATTGTGGTGGCTCCACCGCCTACCTGACTTACGGAT |
| | | 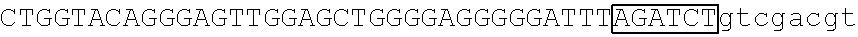 |
| | | gcccagggattgtggttgtaagccttgcatatgtacagtcccagaagtatcatctgtcttcatcttccccccaaagccc |
| | | aaggatgtgctcaccattactctgactcctaaggtcacgtgtgttgtggtagacatcagcaaggatgatcccgaggt |
| | | ccagttcagctggtttgtagatgatgtggaggtgcacacagctcagacgcaaccccgggaggagcagttcaacag |
| | | cactttccgctcagtcagtgaacttcccatcatgcaccaggactggctcaatggcaaggagttcaaatgcagggtc |
| | | aacagtgcagctttccctgcccccatcgagaaaaccatctccaaaaccaaaggcagaccgaaggctccacaggt |
| | | gtacaccattccacctcccaaggagcagatggccaaggataaagtcagtctgacctgcatgataacagacttcttc |
| | | cctgaagacattactgtggagtggcagtggaatgggcagccagcggagaactacaagaacactcagcccatcat |
| | | ggacacagatggctcttacttcgtctacagcaagctcaatgtgcagaagagcaactgggaggcaggaaatactttc |
| | | acctgctctgtgttacatgagggcctgcacaaccaccatactgagaagagcctctcccactctcctggtaaatga |
| 42 | Primer SNV | GGCGGCCGCACCACCATGGACTGTCTCACCAACCTC |
| | (PB101) NotI | |
| | Fwd | |
| 43 | Primer SNV89 | CCCAGATCTAAATCCCCCTCCCCAGCTCCAACT |
| | BglII Rev | |
| 44 | Primer SNV99 | CCCAGATCTAGGTTTAAACACACATTCCCACTGTTG |
| | (Lobe1) BglII | |
| | Rev | |
| 45 | Primer | CCCAGATCTCCGAATCATGTCAGTGGGACC |
| | SNV195 | |
| | (PB101) BglII | |
| | Rev | |
| 46 | Primer | CCCAGATCTACGCACAGACTCTTCCCGAATCAT |
| | SNV200 BgIII | |
| | Rev | |
| 47 | Primer | CCCAGATCTACAAAGCCAGCAGTTCTCTGCTAG |
| | SNV258 | |
| | (PB101) BglII | |
| | Rev | |
| 48 | SNV Env | MDCLTNLRSAEGKVDQASKILILLVAWWGFGTTAEG |
| | signal peptide | |
| 49 | SNV94mFc | MDCLTNLRSAEGKVDQASKILILLVAWWGFGTTAEGYPLQQLWGL |
| | | PCDCSGGYVSSIPTYYTYSLDCGGSTAYLTYGSGTGSWSWGGGFKQ |
| | | 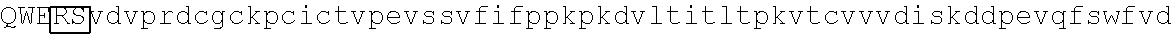 |
| | | dvevhtaqtqpreeqfnstfrsvselpimhqdwlngkefkcrvnsaafpapiektisktkgrpkapqvyti |
| | | pppkeqmakdkvsltcmitdffpeditvewqwngqpaenykntqpimdtdgsyfvysklnvqksn |
| | | weagntftcsvlheglhnhhtekslshspgk |
| 50 | SNV97mFc | MDCLTNLRSAEGKVDQASKILILLVAWWGFGTTAEGYPLQQLWGL |
| | | PCDCSGGYVSSIPTYYTYSLDCGGSTAYLTYGSGTGSWSWGGGFKQ |
| | | 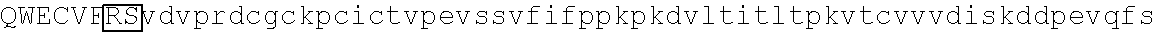 |
| | | wfvddvevhtaqtqpreeqfnstfrsvselpimhqdwlngkefkcrvnsaafpapiektisktkgrpkap |
| | | qvytipppkeqmakdkvsltcmitdffpeditvewqwngqpaenykntqpimdtdgsyfvysklnv |
| | | qksnweagntftcsvlheglhnhhtekslshspgk |
| 51 | SNV98mFc | MDCLTNLRSAEGKVDQASKILILLVAWWGFGTTAEGYPLQQLWGL |
| | | PCDCSGGYVSSIPTYYTYSLDCGGSTAYLTYGSGTGSWSWGGGFKQ |
| | | 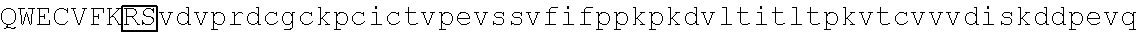 |
| | | fswfvddvevhtaqtqpreeqfnstfrsvselpimhqdwlngkefkcrvnsaafpapiektisktkgrpk |
| | | apqvytipppkeqmakdkvsltcmitdffpeditvewqwngqpaenykntqpimdtdgsyfvyskl |
| | | nvqksnweagntftcsvlheglhnhhtekslshspgk |
| 52 | SNV210mFc | MDCLTNLRSAEGKVDQASKILILLVAWWGFGTTAEGYPLQQLWGL |
| | | PCDCSGGYVSSIPTYYTYSLDCGGSTAYLTYGSGTGSWSWGGGFKQ |
| | | QWECVFKPKIIPSVQGQPGPCPSECLQIATQMHSTCYEKTQECTLLG |
| | | KTYFTAILQKTKLGSYEDGPNKLIQASCTGTVGKPVCWDPVAPVYV |
| | | 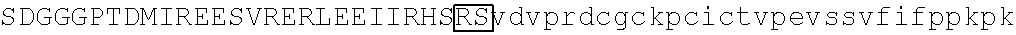 |
| | | dvltitltpkvtcvvvdiskddpevqfswfvddvevhtaqtqpreeqfnstfrsvselpimhqdwlngke |
| | | fkcrvnsaafpapiektisktkgrpkapqvytipppkeqmakdkvsltcmitdffpeditvewqwngq |
| | | paenykntqpimdtdgsyfvysklnvqksnweagntftcsvlheglhnhhtekslshspgk |
| 53 | SNV220mFc | MDCLTNLRSAEGKVDQASKILILLVAWWGFGTTAEGYPLQQLWGL |
| | | PCDCSGGYVSSIPTYYTYSLDCGGSTAYLTYGSGTGSWSWGGGFKQ |
| | | QWECVFKPKIIPSVQGQPGPCPSECLQIATQMHSTCYEKTQECTLLG |
| | | KTYFTAILQKTKLGSYEDGPNKLIQASCTGTVGKPVCWDPVAPVYV |
| | | 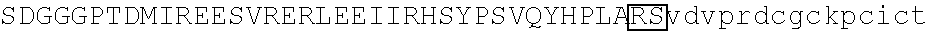 |
| | | vpevssvfifppkpkdvltitltpkvtcvvvdiskddpevqfswfvddvevhtaqtqpreeqfnstfrsvs |
| | | elpimhqdwlngkefkcrvnsaafpapiektisktkgrpkapqvytipppkeqmakdkvsltcmitdff |
| | | peditvewqwngqpaenykntqpimdtdgsyfvysklnvqksnweagntftcsvlheglhnhhteks |
| | | lshspgk |
| 54 | SNV258mFc | MDCLTNLRSAEGKVDQASKILILLVAWWGFGTTAEGYPLQQLWGL |
| | | PCDCSGGYVSSIPTYYTYSLDCGGSTAYLTYGSGTGSWSWGGGFKQ |
| | | QWECVFKPKIIPSVQGQPGPCPSECLQIATQMHSTCYEKTQECTLLG |
| | | KTYFTAILQKTKLGSYEDGPNKLIQASCTGTVGKPVCWDPVAPVYV |
| | | SDGGGPTDMIREESVRERLEEIIRHSYPSVQYHPLALPRSRGVDLDPQ |
| | | 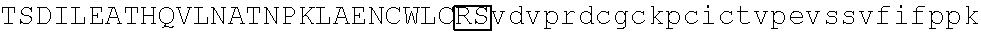 |
| | | pkdvltitltpkvtcvvvdiskddpevqfswfvddvevhtaqtqpreeqfnstfrsvselpimhqdwlng |
| | | kefkcrvnsaafpapiektisktkgrpkapqvytipppkeqmakdkvsltcmitdffpeditvewqwn |
| | | gqpaenykntqpimdtdgsyfvysklnvqksnweagntftcsvlheglhnhhtekslshspgk |
| 55 | SNV94mFc | ATGGACTGTCTCACCAACCTCCGATCCGCTGAGGGTAAAGTTGACC |
| | nucleic acid | AGGCGAGCAAAATCCTAATTCTCCTTGTGGCTTGGTGGGGGTTTGG |
| | | GACCACTGCCGAAGGTTACCCCTTGCAGCAACTTTGGGGACTGCCT |
| | | TGTGACTGTTCCGGGGGATATGTCTCCTCCATACCTACCTATTACAC |
| | | CTACTCCCTCGATTGTGGTGGCTCCACCGCCTACCTGACTTACGGAT |
| | | CTGGTACAGGGAGTTGGAGCTGGGGAGGGGGATTTAAACAACAGT |
| | | 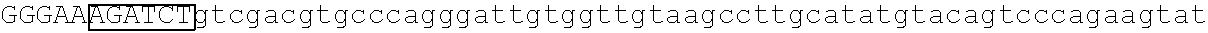 |
| | | catctgtcttcatcttccccccaaagcccaaggatgtgctcaccattactctgactcctaaggtcacgtgtgttgtggta |
| | | gacatcagcaaggatgatcccgaggtccagttcagctggtttgtagatgatgtggaggtgcacacagctcagacg |
| | | caaccccgggaggagcagttcaacagcactttccgctcagtcagtgaacttcccatcatgcaccaggactggctc |
| | | aatggcaaggagttcaaatgcagggtcaacagtgcagctttccctgcccccatcgagaaaaccatctccaaaacc |
| | | aaaggcagaccgaaggctccacaggtgtacaccattccacctcccaaggagcagatggccaaggataaagtca |
| | | gtctgacctgcatgataacagacttcttccctgaagacattactgtggagtggcagtggaatgggcagccagcgga |
| | | gaactacaagaacactcagcccatcatggacacagatggctcttacttcgtctacagcaagctcaatgtgcagaag |
| | | agcaactgggaggcaggaaatactttcacctgctctgtgttacatgagggcctgcacaaccaccatactgagaaga |
| | | gcctctcccactctcctggtaaatga |
| 56 | SNV97mFc | ATGGACTGTCTCACCAACCTCCGATCCGCTGAGGGTAAAGTTGACC |
| | nucleic acid | AGGCGAGCAAAATCCTAATTCTCCTTGTGGCTTGGTGGGGGTTTGG |
| | | GACCACTGCCGAAGGTTACCCCTTGCAGCAACTTTGGGGACTGCCT |
| | | TGTGACTGTTCCGGGGGATATGTCTCCTCCATACCTACCTATTACAC |
| | | CTACTCCCTCGATTGTGGTGGCTCCACCGCCTACCTGACTTACGGAT |
| | | CTGGTACAGGGAGTTGGAGCTGGGGAGGGGGATTTAAACAACAGT |
| | | 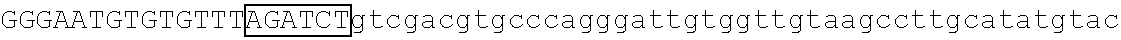 |
| | | agtcccagaagtatcatctgtcttcatcttccccccaaagcccaaggatgtgctcaccattactctgactcctaaggtc |
| | | acgtgtgttgtggtagacatcagcaaggatgatcccgaggtccagttcagctggtttgtagatgatgtggaggtgca |
| | | cacagctcagacgcaaccccgggaggagcagttcaacagcactttccgctcagtcagtgaacttcccatcatgca |
| | | ccaggactggctcaatggcaaggagttcaaatgcagggtcaacagtgcagctttccctgcccccatcgagaaaac |
| | | catctccaaaaccaaaggcagaccgaaggctccacaggtgtacaccattccacctcccaaggagcagatggcca |
| | | aggataaagtcagtctgacctgcatgataacagacttcttccctgaagacattactgtggagtggcagtggaatggg |
| | | cagccagcggagaactacaagaacactcagcccatcatggacacagatggctcttacttcgtctacagcaagctc |
| | | aatgtgcagaagagcaactgggaggcaggaaatactttcacctgctctgtgttacatgagggcctgcacaaccac |
| | | catactgagaagagcctctcccactctcctggtaaatga |
| 57 | SNV98mFc | ATGGACTGTCTCACCAACCTCCGATCCGCTGAGGGTAAAGTTGACC |
| | nucleic acid | AGGCGAGCAAAATCCTAATTCTCCTTGTGGCTTGGTGGGGGTTTGG |
| | | GACCACTGCCGAAGGTTACCCCTTGCAGCAACTTTGGGGACTGCCT |
| | | TGTGACTGTTCCGGGGGATATGTCTCCTCCATACCTACCTATTACAC |
| | | CTACTCCCTCGATTGTGGTGGCTCCACCGCCTACCTGACTTACGGAT |
| | | CTGGTACAGGGAGTTGGAGCTGGGGAGGGGGATTTAAACAACAGT |
| | | 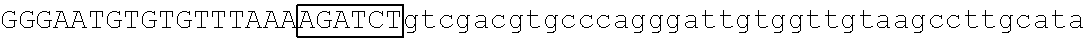 |
| | | tgtacagtcccagaagtatcatctgtcttcatcttccccccaaagcccaaggatgtgctcaccattactctgactccta |
| | | aggtcacgtgtgttgtggtagacatcagcaaggatgatcccgaggtccagttcagctggtttgtagatgatgtggag |
| | | gtgcacacagctcagacgcaaccccgggaggagcagttcaacagcactttccgctcagtcagtgaacttcccatc |
| | | atgcaccaggactggctcaatggcaaggagttcaaatgcagggtcaacagtgcagctttccctgcccccatcgag |
| | | aaaaccatctccaaaaccaaaggcagaccgaaggctccacaggtgtacaccattccacctcccaaggagcagat |
| | | ggccaaggataaagtcagtctgacctgcatgataacagacttcttccctgaagacattactgtggagtggcagtgg |
| | | aatgggcagccagcggagaactacaagaacactcagcccatcatggacacagatggctcttacttegtctacagc |
| | | aagctcaatgtgcagaagagcaactgggaggcaggaaatactttcacctgctctgtgttacatgagggcctgcaca |
| | | accaccatactgagaagagcctctcccactctcctggtaaatga |
| 58 | SNV210mFc | ATGGACTGTCTCACCAACCTCCGATCCGCTGAGGGTAAAGTTGACC |
| | nucleic acid | AGGCGAGCAAAATCCTAATTCTCCTTGTGGCTTGGTGGGGGTTTGG |
| | | GACCACTGCCGAAGGTTACCCCTTGCAGCAACTTTGGGGACTGCC |
| | | TTGTGACTGTTCCGGGGGATATGTCTCCTCCATACCTACCTATTAC |
| | | ACCTACTCCCTCGATTGTGGTGGCTCCACCGCCTACCTGACTTAC |
| | | GGATCTGGTACAGGGAGTTGGAGCTGGGGAGGGGGATTTAAACA |
| | | ACAGTGGGAATGTGTGTTTAAACCTAAGATCATACCCTCTGTGCA |
| | | GGGGCAGCCAGGGCCCTGCCCATCTGAATGCCTCCAGATAGCTA |
| | | CTCAAATGCATTCCACTTGTTATGAAAAGACTCAGGAATGCACCC |
| | | TCCTGGGAAAAACCTATTTTACTGCCATCCTACAGAAAACAAAGC |
| | | TAGGCTCGTATGAAGACGGGCCTAATAAACTGATCCAGGCCTCTT |
| | | GCACGGGAACCGTAGGGAAACCAGTATGTTGGGACCCTGTAGCC |
| | | CCTGTGTATGTCTCTGATGGCGGCGGTCCCACTGACATGATTCGG |
| | | GAAGAGTCTGTGCGTGAAAGACTAGAGGAAATCATCAGGCACAG |
| | | 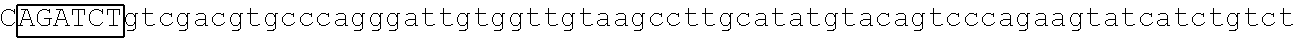 |
| | | tcatcttccccccaaagcccaaggatgtgctcaccattactctgactcctaaggtcacgtgtgttgtggtagacatca |
| | | gcaaggatgatcccgaggtccagttcagctggtttgtagatgatgtggaggtgcacacagctcagacgcaacccc |
| | | gggaggagcagttcaacagcactttccgctcagtcagtgaacttcccatcatgcaccaggactggctcaatggcaa |
| | | ggagttcaaatgcagggtcaacagtgcagctttccctgcccccatcgagaaaaccatctccaaaaccaaaggcag |
| | | accgaaggctccacaggtgtacaccattccacctcccaaggagcagatggccaaggataaagtcagtctgacctg |
| | | catgataacagacttcttccctgaagacattactgtggagtggcagtggaatgggcagccagcggagaactacaa |
| | | gaacactcagcccatcatggacacagatggctcttacttcgtctacagcaagctcaatgtgcagaagagcaactgg |
| | | gaggcaggaaatactttcacctgctctgtgttacatgagggcctgcacaaccaccatactgagaagagcctctccc |
| | | actctcctggtaaatga |
| 59 | SNV220mFc | ATGGACTGTCTCACCAACCTCCGATCCGCTGAGGGTAAAGTTGACC |
| | nucleic acid | AGGCGAGCAAAATCCTAATTCTCCTTGTGGCTTGGTGGGGGTTTGG |
| | | GACCACTGCCGAAGGTTACCCCTTGCAGCAACTTTGGGGACTGCC |
| | | TTGTGACTGTTCCGGGGGATATGTCTCCTCCATACCTACCTATTAC |
| | | ACCTACTCCCTCGATTGTGGTGGCTCCACCGCCTACCTGACTTAC |
| | | GGATCTGGTACAGGGAGTTGGAGCTGGGGAGGGGGATTTAAACA |
| | | ACAGTGGGAATGTGTGTTTAAACCTAAGATCATACCCTCTGTGCA |
| | | GGGGCAGCCAGGGCCCTGCCCATCTGAATGCCTCCAGATAGCTA |
| | | CTCAAATGCATTCCACTTGTTATGAAAAGACTCAGGAATGCACCC |
| | | TCCTGGGAAAAACCTATTTTACTGCCATCCTACAGAAAACAAAGC |
| | | TAGGCTCGTATGAAGACGGGCCTAATAAACTGATCCAGGCCTCTT |
| | | GCACGGGAACCGTAGGGAAACCAGTATGTTGGGACCCTGTAGCC |
| | | CCTGTGTATGTCTCTGATGGCGGCGGTCCCACTGACATGATTCGG |
| | | GAAGAGTCTGTGCGTGAAAGACTAGAGGAAATCATCAGGCACAG |
| | | 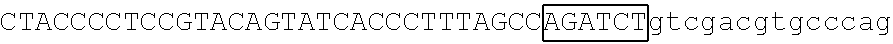 |
| | | ggattgtggttgtaagccttgcatatgtacagtcccagaagtatcatctgtcttcatcttccccccaaagcccaaggat |
| | | gtgctcaccattactctgactcctaaggtcacgtgtgttgtggtagacatcagcaaggatgatcccgaggtccagttc |
| | | agctggtttgtagatgatgtggaggtgcacacagctcagacgcaaccccgggaggagcagttcaacagcactttc |
| | | cgctcagtcagtgaacttcccatcatgcaccaggactggctcaatggcaaggagttcaaatgcagggtcaacagt |
| | | gcagctttccctgcccccatcgagaaaaccatctccaaaaccaaaggcagaccgaaggctccacaggtgtacac |
| | | cattccacctcccaaggagcagatggccaaggataaagtcagtctgacctgcatgataacagacttcttccctgaa |
| | | gacattactgtggagtggcagtggaatgggcagccagcggagaactacaagaacactcagcccatcatggacac |
| | | agatggctcttacttcgtctacagcaagctcaatgtgcagaagagcaactgggaggcaggaaatactttcacctgct |
| | | ctgtgttacatgagggcctgcacaaccaccatactgagaagagcctctcccactctcctggtaaatga |
| 60 | SNV258mFc | ATGGACTGTCTCACCAACCTCCGATCCGCTGAGGGTAAAGTTGACC |
| | nucleic acid | AGGCGAGCAAAATCCTAATTCTCCTTGTGGCTTGGTGGGGGTTTGG |
| | | GACCACTGCCGAAGGTTACCCCTTGCAGCAACTTTGGGGACTGCC |
| | | TTGTGACTGTTCCGGGGGATATGTCTCCTCCATACCTACCTATTAC |
| | | ACCTACTCCCTCGATTGTGGTGGCTCCACCGCCTACCTGACTTAC |
| | | GGATCTGGTACAGGGAGTTGGAGCTGGGGAGGGGGATTTAAACA |
| | | ACAGTGGGAATGTGTGTTTAAACCTAAGATCATACCCTCTGTGCA |
| | | GGGGCAGCCAGGGCCCTGCCCATCTGAATGCCTCCAGATAGCTA |
| | | CTCAAATGCATTCCACTTGTTATGAAAAGACTCAGGAATGCACCC |
| | | TCCTGGGAAAAACCTATTTTACTGCCATCCTACAGAAAACAAAGC |
| | | TAGGCTCGTATGAAGACGGGCCTAATAAACTGATCCAGGCCTCTT |
| | | GCACGGGAACCGTAGGGAAACCAGTATGTTGGGACCCTGTAGCC |
| | | CCTGTGTATGTCTCTGATGGCGGCGGTCCCACTGACATGATTCGG |
| | | GAAGAGTCTGTGCGTGAAAGACTAGAGGAAATCATCAGGCACAG |
| | | CTACCCCTCCGTACAGTATCACCCTTTAGCCCTGCCCCGATCAAG |
| | | AGGAGTAGATCTGGATCCCCAGACGTCTGACATACTGGAAGCTA |
| | | CTCACCAGGTCCTTAATGCCACTAATCCCAAGCTAGCAGAGAACT |
| | | 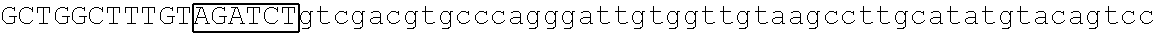 |
| | | cagaagtatcatctgtcttcatcttccccccaaagcccaaggatgtgctcaccattactctgactcctaaggtcacgtg |
| | | tgttgtggtagacatcagcaaggatgatcccgaggtccagttcagctggtttgtagatgatgtggaggtgcacacag |
| | | ctcagacgcaaccccgggaggagcagttcaacagcactttccgctcagtcagtgaacttcccatcatgcaccagg |
| | | actggctcaatggcaaggagttcaaatgcagggtcaacagtgcagctttccctgcccccatcgagaaaaccatctc |
| | | caaaaccaaaggcagaccgaaggctccacaggtgtacaccattccacctcccaaggagcagatggccaaggat |
| | | aaagtcagtctgacctgcatgataacagacttcttccctgaagacattactgtggagtggcagtggaatgggcagc |
| | | cagcggagaactacaagaacactcagcccatcatggacacagatggctcttacttcgtctacagcaagctcaatgt |
| | | gcagaagagcaactgggaggcaggaaatactttcacctgctctgtgttacatgagggcctgcacaaccaccatact |
| | | gagaagagcctctcccactctcctggtaaatga |

The present invention is further illustrated by the following examples.

### EXAMPLES

### Example 1: a Polypeptide Ligand that Specifically Binds ASCT1/SLC1A4 Distinctively from ASCT2/SLC1A5

### Materials and Methods

### Cells

CHO (chinese hamster ovary) cells were cultured in F12 Nut Mix (Ham) (Ham's F-12 Nutrient Mix) culture medium (Gibco, 21765-029) with 10% decomplemented fetal bovine serum (Sigma-Aldrich, F7524); L-glutamine (Gibco, 25030-024); and 1% antibiotics (Penicillin and Streptomycin). Cells were cultivated under humid atmosphere in a 5% CO₂ incubator at 37° C.

### Plasmids

RBDs of different lengths were generated and amplified by PCR. All primers were provided by Integrated DNA Technology (Table 1).

Amplification was performed in a final volume of 50 μl with Q5 High-Fidelity DNA Polymerase (New England BioLabs, M0491S), 0.5 mM of each deoxyribonucleoside triphosphate (dATP, dCTP, dGTP, dTTP), and 0.5 μM of each primer. DNA was denatured for 2 min at 94° C., followed by 30 cycles of amplification: 30 sec at 94° C.; 1 min at specified hybridization temperature for each RBD and 30 sec at 68° C.; and final elongation for 6 min at 68° C. PCR products were then separated on 0.8% agarose gels.

After sequencing and validation, the fragments were inserted into the final expression vector pCSI-mFc, which has a 3′ tag encoding the Fc fragment of mouse IgG1.

TABLE 1
List of primers

| | | SEQ ID |
|---|---|---|
| Primer name | Sequence | NO |
| SNV (PB101) NotI Fwd | GGCGGCCGCACCACCATGGACTGTCTCAC | 42 |
| | CAACCTC | |
| SNV89 BglII Rev | CCCAGATCTAAATCCCCCTCCCCAGCTCCA | 43 |
| | ACT | |
| SNV99 (Lobe1) BglII Rev | CCCAGATCTAGGTTTAAACACACATTCCC | 44 |
| | ACTGTTG | |
| SNV195 (PB101) BglII Rev | CCCAGATCTCCGAATCATGTCAGTGGGAC | 45 |
| | C | |
| SNV200 BglII Rev | CCCAGATCTACGCACAGACTCTTCCCGAA | 46 |
| | TCAT | |
| SNV258 (PB101) BglII Rev | CCCAGATCTACAAAGCCAGCAGTTCTCTG | 47 |
| | CTAG | |

### Production of RBD Ligands

RBD ligand production was performed according to the following protocol: RBD expression vectors (38.5 μg) were transfected into HEK293T cells (8×106 cells) grown on poly-D-lysine-coated (Sigma-Aldrich, P7405) T175 flask using the PEIpro® transfection reagent (38.5 μl) (Polyplus+Transfection, 115-010). The medium was changed 6 hours post-transfection and replaced with OptiPRO SFM serum-free medium (Gibco, 12309-019) supplemented with non-essential amino acids (Gibco, 11140-035), L-glutamine (Gibco, 25030-024) and antibiotics (Penicillin and Streptomycin). 72 hours post-transfection, the culture medium was harvested and filtered through 0.45 μm filters. The supernatant was then concentrated 100-fold by centrifugation (3800 rpm at 4° C.) using Amicon Ultra-15 30 kDa concentration tubes (Merck Millipore, UFC903024). The RBDs produced were then stored at −20° C. To determine the optimal amount of ligand usage, binding saturation curves were performed using flow cytometry for each batch produced on 1×10⁵ HEK293T cells.

### Testing the Specificity of the RBDs on Transient Transporter Overexpression by Flow Cytometry

Chinese hamster ovary (CHO) cells (2.5×105 cells/well) grown on 6-well plates were transfected with 2 μg of empty expression vector or expression vector with the DNA sequence coding for human SLC transporters using the jetPRIME® transfection method (200 μl of jetPRIMER buffer and 6 μl of jetPRIME® reagent) (Polyplus+Transfection, 114-015). The SLCs used are fused with HA or 3×Flag tags in their C-terminal side. It is therefore possible to monitor the SLC overexpression and to assess the specificity of the RBDs by flow cytometry 48 hours after transfection.

For RBD labeling, cells were detached with PBS containing trypsin-EDTA. 1×10⁵ cells were used per labeling and resuspended in 50 μl of PBA (PBS with 1% FBS) with the saturating dilution of RBD. Cells were incubated for 30 min at 37° C., washed twice with PBA, and incubated for 20 min at 4° C. with an R-phycoerythrin goat anti-mouse IgG1 conjugated antibody (1:250, Invitrogen, P21129). After two washes, cells were ready for acquisition with Novocyte (Acea, Biosciences, Inc). Data analysis was performed using FlowJo V10 software.

For SLC labeling, 1×10⁵ cells were fixed and permeabilized with the BD Cytofix/Cytoperm kit (BD Biosciences 554722) following the supplier's protocol. After permeabilization the cells were incubated for 20 min with an anti-HA-fluorescein antibody (Roche, 11988506001) or an anti-Flag M2-FITC antibody (Sigma-Aldrich, F4049) diluted 250-fold. After two washes, cells were promptly analyzed on Novocyte (Acea, Biosciences, Inc) and data were analyzed with the FlowJo V10 software.

### Statistical Analysis

Statistical analyses were performed using Student's t-test in GraphPad Prism 5 software (GraphPad Software Inc., La Jolla, CA, USA). The data were expressed as the mean±SEM. The results were considered statistically significant at a p-value<0.05 (*), <0.01 (**) or <0.001 (***).

### Results

SNV195, which corresponds to the first 195 amino acid residues of the SNV RBD starting from the N-terminal end, was generated and fused to a mouse Fc (corresponding to amino acid sequence SEQ ID NO: 35, encoded by a nucleic acid sequence as set forth in SEQ ID NO: 39). The binding specificity of SNV195 towards human ASCT1/SLC1A4, ASCT2/SLC1A5 and EAAT3/SLC1A was assessed by flow cytometry using vector transfection-mediated overexpression models.

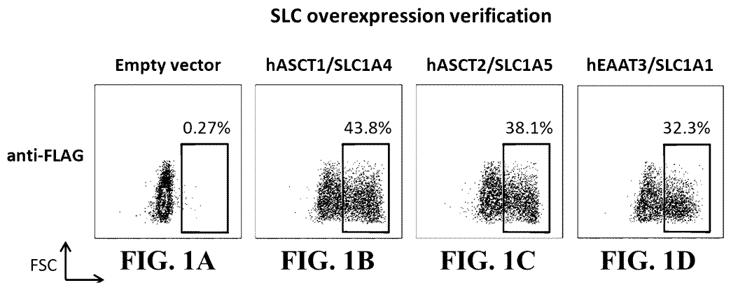

Overexpression transient models were performed in Chinese hamster ovary (CHO) cells, which presented low, if any, RBD background binding. An empty vector, as well as expression vectors of members of the SLC1A family carrying a FLAG tag, namely, hASCT1/SLC1A4, hASCT2/SLC1A5, hEAAT3/SLC1A1 were overexpressed by transient transfection. SLC overexpression was checked by flow cytometry using an anti-FLAG antibody. As shown on FIG. 1A-D, overexpression of hASCT1/SLC1A4 (FIG. 1B), hASCT2/SLC1A5 (FIG. 1C) and hEAAT3/SLC1A1 (FIG. 1D) gave equivalent levels of expression for each SLC, with 43.8%, 38.1% and 32.3% of transfected cells, respectively. While no overexpression was detected in cells transfected with an empty vector (FIG. 1A).

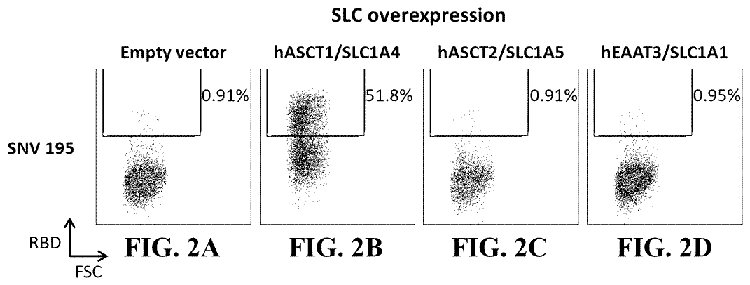

SNV195 was fused to a mouse Fc, and its binding was assessed by flow cytometry using an anti-mouse IgG1 conjugated antibody. Overexpression of the hASCT1/SLC1A4 transporter resulted in an increased binding signal of SNV195 (FIG. 2B), while overexpression of the empty vector (FIG. 2A), hASCT2/SLC1A5 (FIG. 2C) or hEAAT3/SLC1A1 (FIG. 2D) transporters did not change the low background binding signal of this RBD, which indicates that SNV195 specifically recognizes hASCT1/SLC1A4.

SNV RBDs of different lengths were generated and fused to a mouse Fc: SNV89 (corresponding to amino acid sequence SEQ ID NO: 37, encoded by a nucleic acid sequence as set forth in SEQ ID NO: 41), SNV99 (corresponding to amino acid sequence SEQ ID NO: 36, encoded by a nucleic acid sequence as set forth in SEQ ID NO: 40), SNV195 (corresponding to amino acid sequence SEQ ID NO: 35, encoded by a nucleic acid sequence as set forth in SEQ ID NO: 39) and SNV200 (corresponding to amino acid sequence SEQ ID NO: 34, encoded by a nucleic acid sequence as set forth in SEQ ID NO: 38), and their binding specificity was evaluated in CHO overexpressing hASCT1/SLC1A4 and hASCT2/SLC1A5 transporters.

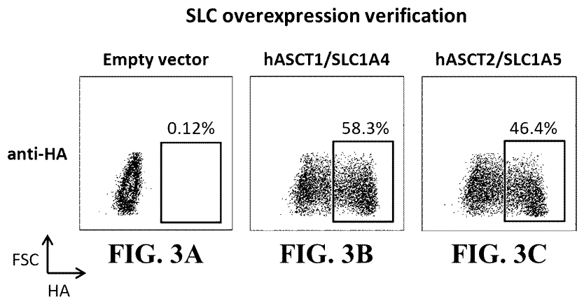

As described above, overexpression transient models were performed in CHO cells using an empty vector, as well as expression vectors of hASCT1/SLC1A4 and hASCT2/SLC1A5 carrying an HA tag. SLC overexpression was checked by flow cytometry using an anti-HA antibody. As shown on FIG. 3A-C, overexpression of hASCT1/SLC1A4 (FIG. 3B) and hASCT2/SLC1A5 (FIG. 3C) gave equivalent levels of expression for both SLC, with 58.3% and 46.4% of transfected cells respectively. While no overexpression was detected in cells transfected with an empty vector (FIG. 3A).

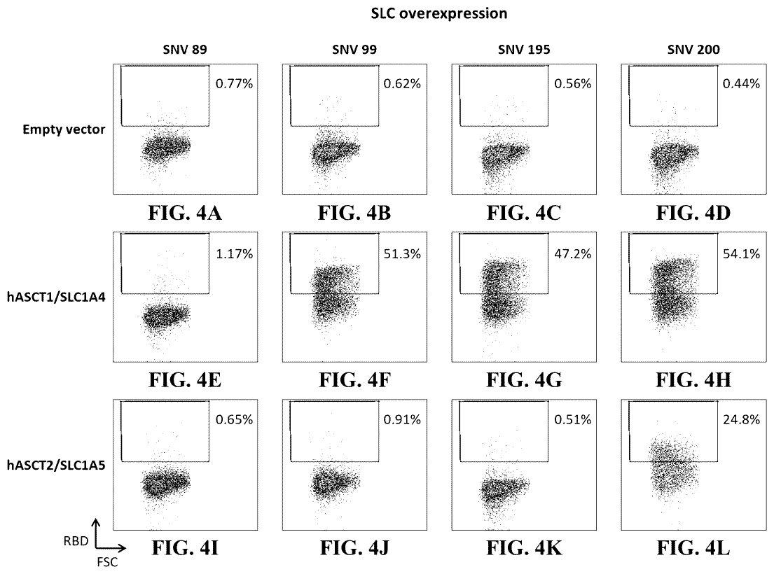

Each SNV RBD was fused to a mouse Fc, and binding was assessed by flow cytometry using an anti-mouse IgG1 conjugated antibody. No binding was detected on the empty vector, which was used as a control, with either of the SNV construct (FIG. 4A-D). Dual recognition of hASCT1/SLC1A4 and hASCT2/SLC1A5 was observed with SNV200 RBD (FIGS. 4H and 4L). Binding towards hASCT1/SLC1A4 was obtained with SNV195 (FIG. 4G) and SNV99 RBD (FIG. 4F), while there was no binding towards hASCT2/SLC1A5 for either RBD construct (FIGS. 4J and 4K). This demonstrate that constructs comprising between residues 99 and 195 are able to bind specifically to hASCT1/SLC1A4, and not hASCT2/SLC1A5. Finally, no binding was detected with SNV89 RBD, neither towards hASCT1/SLC1A4 (FIG. 4E), nor towards hASCT2/SLC1A5 (FIG. 4I). This indicates that the carboxy terminal limit of the SNV domain for specific and distinctive recognition of hASCT1/SLC1A4 is comprised between amino acid residues 90 and 98.

### Example 2: SNV 195 Specifically Impairs the Serine Transport Function of ASCT1/SLC1A4

### Materials and Methods

### Glutamine- and Serine-Uptake Assays

Serine and glutamine uptake were measured in cell monolayers seeded at 1-5×10⁵ cells per well in 24-well plates. One day later the culture medium was aspirated and cells were preincubated for 30 min at 37° C. in the uptake buffer containing the RBD serially-diluted in modified DMEM, without glucose, glutamine, serine, glycine culture medium (Biological Industries, 06-1056-08-1A) supplemented with 0.3 mM L-glutamine (Gibco, 25030-024) and 0.5 g/L D-Glucose (Gibco, A24940-01) for serine uptake assays; and supplemented with 0.1 mM serine (Gibco, 11140-035) and 0.5 g/L D-Glucose (Gibco, A24940-01) for glutamine uptake assays. After the cells were incubated for 5 min at 37° C. in fresh uptake buffer supplemented with 1 μCi/ml [3H]-serine or [3H]-glutamine (NET248250UC and NET551250UC respectively, PerkinElmer).

Uptake was terminated by rapid removal of the medium followed by three washes with ice-cold phosphate-buffered saline (PBS). Cells were lysed 1 hour in SDS 10% prior to analysis for radioactivity by scintillation counting and for protein content by the Pierce BCA Protein Assay Kit (Thermo Scientific, 23225). For counting, samples were mixed with 2 ml of scintillation fluid (ULTIMA GOLD, Perkin Elmer) and analyzed with the Hidex 300 SL instrument. The percentage of serine or glutamine uptake was calculated as the ratio of cellular [3H]-serine or [3H]-glutamine to total [3H]-serine or [3H]-glutamine supplemented. Each uptake was represented as the mean of 3-5 replicates and each experiment was repeated 2-4 times.

### Results

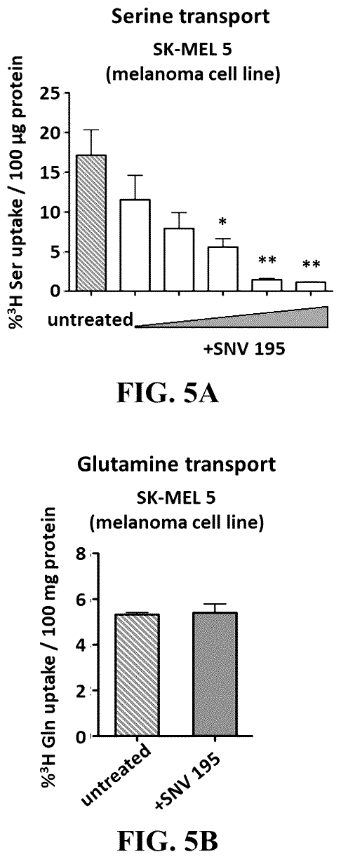

In addition to specifically binding its cognate receptor (i.e., ASCT1/SLC1A4), SNV195 was also able to alter its transport function. This is shown on FIG. 5A, where a dramatic decrease of cellular serine uptake is observed upon addition of increasing concentrations of SNV195 (corresponding to amino acid sequence SEQ ID NO: 35, encoded by a nucleic acid sequence as set forth in SEQ ID NO: 39) in the culture medium of melanoma SK-MEL 5 cells treated with radiolabeled [3H]-serine. The fact that SNV195 has no impact on cellular glutamine uptake, as shown on FIG. 5B, provides further evidence of its specificity. Glutamine is not an ASCT1 substrate, while it is the major substrate of ASCT2, which shares 57% identity and almost an overlapping 3D architecture with ASCT1.

These results further demonstrate the specific binding of SNV195 towards ASCT1/SLC1A4, and shows SNV195 functional impact on ASCT1/SLC1A4. Additionally, the capacity of SNV195 to block ASCT1 transport function indicates that SNV195 has the potential be used as a therapeutic tool.

### Example 3: SNV195 Allows the Specific Detection of ASCT1 in IHC of Solid Tumors

### Materials and Methods

### Immunohistochemistry Analyses

Human tissue micro array (TMA) thin sections (BioChain, catalog No. T8235713-5, Lot No. B306119) containing 94 cores of healthy and tumor tissue were immunostained with SNV195 RBD according to the following procedure: paraffin-embedded tissue sections were deparaffinised with xylene and graded ethanol, and antigen retrieval was performed by heating the sections in EDTA buffer, pH 9, for 20 min at 98.5° C. Endogenous peroxidase were blocked with EnVision™ FLEX Peroxidase-Blocking Reagent (Dako, DM841), followed by a blocking step performed with 20% goat serum prior to incubation for 30 min at 37° C. with either the SNV195 RBD diluted 1500 times, or an equivalent dilution of a control RBD preparation. All other steps were performed according to the supplier's recommendations (Dako EnVision FLEX systems kit, ref GV823). The slides were then counterstained with hematoxylin, and then dehydrated and mounted. The slides were scanned with NANOZOOMER 1 HAMAMATSU and images were realized using NDP.view2 at several magnifications.

### Results

The capacity of SNV195 RBD to recognize its target (i.e., ASCT1/SLC1A4) in human tissue was tested by immunohistochemistry (IHC) staining.

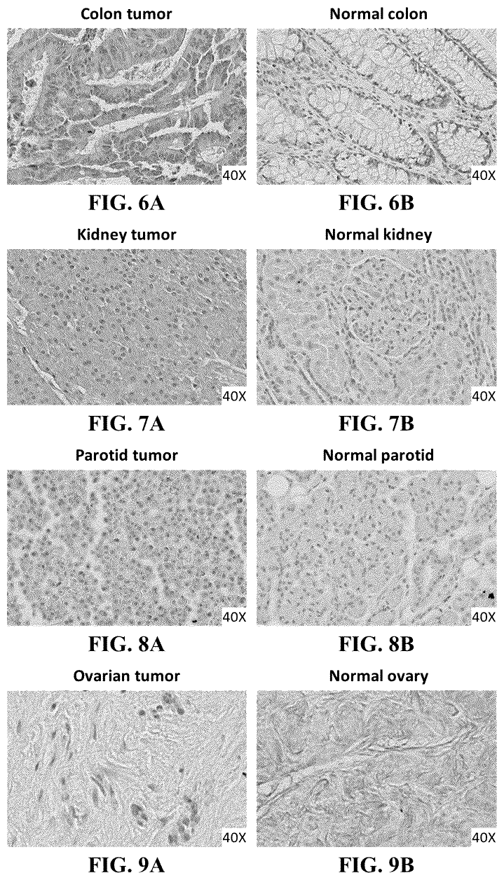

We tested SNV195 RBD by IHC on tissue microarrays (TMA) of healthy and tumor tissues. A strong staining in the tumor tissue of colon (FIG. 6A), kidney (FIG. 7A) and parotid (FIG. 8A) was observed with no or low staining in corresponding healthy tissues (FIGS. 6B, 7B and 8B). Conversely, strong staining in normal tissue (FIG. 9B) with no staining in tumor tissue (FIG. 9A) was observed in the ovary.

Taken all together, these results indicate that SNV195 RBD can be used as a diagnostic tool for cancer detection and/or grading.

### Example 4: Identification of the Minimal Sequence of SNV RBD Necessary for Specific and Distinctive Recognition of SLC1A4/ASCT1

### Materials and Methods

### Cells

CHO cells were cultured as indicated above in Example 1.

### Plasmids

RBDs of different lengths were generated as indicated above in Example 1.

### Production of RBD Ligands

RBD ligand production was performed as indicated above in Example 1.

### Testing the Specificity of the RBDs on Transient Transporter Overexpression by Flow Cytometry

CHO cells were grown and transfected with empty vector or expression vector for human SLC1A4 or human SLC1A5 as indicated above in Example 1.

For RBD labeling, cells were detached with PBS containing trypsin-EDTA. 1×10⁵ cells were used per labeling and resuspended in 50 μl of PBA (PBS with 1% FBS) with the saturating dilution of RBD. Cells were incubated for 30 min at 37° C., washed twice with PBA, and incubated for 20 min at 4° C. with an R-phycoerythrin goat anti-mouse IgG1 conjugated antibody (1:250, Invitrogen, P21129) or an AlexaFluor™M647 goat anti-mouse IgG1 conjugated antibody (1:250, Invitrogen, A21240). After two washes, cells were ready for acquisition with Novocyte (Acea, Biosciences, Inc). Data analysis was performed using FlowJo V10 software.

### Results

SNV RBDs of different lengths were generated and fused to a mouse Fc: SNV94 (corresponding to amino acid sequence SEQ ID NO: 49, encoded by a nucleic acid sequence as set forth in SEQ ID NO: 55), SNV97 (corresponding to amino acid sequence SEQ ID NO: 50, encoded by a nucleic acid sequence as set forth in SEQ ID NO: 56), SNV98 (corresponding to amino acid sequence SEQ ID NO: 51, encoded by a nucleic acid sequence as set forth in SEQ ID NO: 57), SNV210 (corresponding to amino acid sequence SEQ ID NO: 52, encoded by a nucleic acid sequence as set forth in SEQ ID NO: 58), SNV220 (corresponding to amino acid sequence SEQ ID NO: 53, encoded by a nucleic acid sequence as set forth in SEQ ID NO: 59), and SNV258 (corresponding to amino acid sequence SEQ ID NO: 54, encoded by a nucleic acid sequence as set forth in SEQ ID NO: 60), and their binding specificity was evaluated in CHO overexpressing hASCT1/SLC1A4 and hASCT2/SLC1A5 transporters.

As described above in Example 1, overexpression transient models were performed in CHO cells using an empty vector, as well as expression vectors of hASCT1/SLC1A4 and hASCT2/SLC1A5 carrying an HA tag.

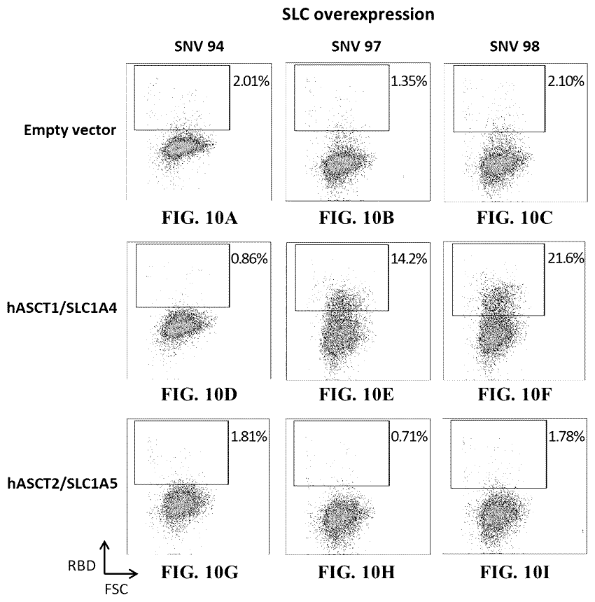

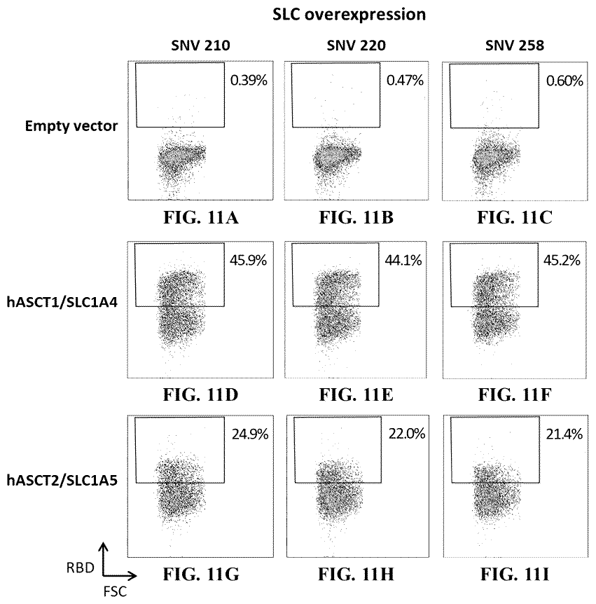

Each SNV RBD was fused to a mouse Fc, and binding towards hASCT1/SLC1A4 and hASCT2/SLC1A5 was assessed by flow cytometry using an anti-mouse IgG1 conjugated antibody. No binding was detected on the empty vector, which was used as a control, with either of the SNV construct (FIGS. 10A-C and FIGS. 11A-C).

Dual recognition of hASCT1/SLC1A4 and hASCT2/SLC1A5 was observed with SNV210 RBD (FIGS. 11D and 11G), as well as with SNV220 RBD (FIGS. 11E and 11H) and with SNV258 RBD (FIGS. 11F and 11I). As indicated above, SNV200 RBD was able to bind both hASCT1/SLC1A4 and hASCT2/SLC1A5 (FIGS. 4H and 4L). This thus confirms that constructs comprising more than 200 amino acid residues from the SNV retroviral envelope glycoprotein, counting from the first methionine of the signal peptide, specifically bind to both hASCT1/SLC1A4 and hASCT2/SLC1A5.

Specific binding towards hASCT1/SLC1A4 was observed with SNV97 RBD (FIG. 10E) and SNV98 RBD (FIG. 10F), while there was no binding towards hASCT2/SLC1A5 for either RBD construct (FIGS. 10H and 10I). Finally, no binding was detected with SNV94 RBD, neither towards hASCT1/SLC1A4 (FIG. 10D), nor towards hASCT2/SLC1A5 (FIG. 10G). This indicates that the carboxy terminal limit of the SNV domain for specific and distinctive recognition of hASCT1/SLC1A4 is comprised between amino acid residues 95 and 97.

1-15. (canceled)

16. A polypeptide of sequence SEQ ID NO: 1 or a functional variant thereof, which specifically binds to ASCT1 and does not specifically bind to ASCT2, wherein:
SEQ ID NO: 1 consists of (X)ₙ-SEQ ID NO: 2-(Y)ₘ (Formula (A));
(X)ₙ is a sequence of n amino acids;
(Y)ₘ is a sequence of m amino acids;
“—” designates a peptide bound;
n≥0 and m≥0; and
if m≥1 then (Y)ₘ does not comprise or consist of the m first amino acids of the sequence SEQ ID NO: 3 or a functional variant thereof, said functional variant of the m first amino acids of the sequence SEQ ID NO: 3 being such that SEQ ID NO: 2-(Y)ₘ specifically binds to ASCT2; and
wherein the functional variant of SEQ ID NO: 1 has a sequence comprising a functional variant of SEQ ID NO: 2 instead of SEQ ID NO: 2, wherein said functional variant of SEQ ID NO: 2 specifically binds to ASCT1.

17. The polypeptide or functional variant thereof according to claim 16, wherein the length of said polypeptide or a functional variant thereof is of less than 100 amino acids, less than 90 amino acids, less than 80 amino acids, less than 70 amino, or less than 64 amino acids.

18. The polypeptide or functional variant thereof according to claim 16, wherein said polypeptide or functional variant has a sequence comprising SEQ ID NO: 4, SEQ ID NO: 5, SEQ ID NO: 6 or SEQ ID NO: 7.

19. The polypeptide or functional variant thereof according to claim 16, wherein m=0.

20. The polypeptide or functional variant thereof according to claim 16, wherein n=0 and m=0.

21. The polypeptide or functional variant thereof according to claim 16, wherein said functional variant of the sequence SEQ ID NO: 1 has a sequence at least 80%, 85%, 90%, 95%, or 99% identical to the sequence SEQ ID NO: 1.

22. The polypeptide or functional variant thereof according to claim 16, wherein said polypeptide or functional variant has a sequence consisting of SEQ ID NO: 2.

23. The polypeptide or functional variant thereof according to claim 16, wherein said polypeptide or functional variant thereof is labeled with a detectable label or is coupled with a contrast agent.

24. A nucleic acid encoding the polypeptide or functional variant thereof according to claim 16, or an expression vector comprising said nucleic acid, or a cell comprising said nucleic acid or said expression vector.

25. A diagnostic or pharmaceutical composition comprising the polypeptide or functional variant thereof according to claim 16, or a nucleic acid encoding said polypeptide or functional variant thereof, or an expression vector comprising said nucleic acid, or a cell comprising said nucleic acid or said expression vector, and at least one pharmaceutically acceptable excipient.

26. An in vitro method of specifically detecting or/and measuring the level of ASCT1 in a sample, while not detecting or/and measuring the level of ASCT2, wherein said method comprises the steps of:
a) contacting said sample with the polypeptide or functional variant thereof according to claim 16, and
b) detecting and/or measuring the binding of said polypeptide or functional variant thereof to ASCT1.

27. A method of specifically detecting or/and measuring the level of ASCT1 in vivo in a subject, while not detecting or/and measuring the level of ASCT2, said method comprising administering to said subject the polypeptide or functional variant thereof according to claim 16.

28. A method of specifically detecting or/and measuring the level of ASCT1 in vitro in a subject, while not detecting or/and measuring the level of ASCT2, said method comprising administering to said subject the polypeptide or functional variant thereof according to claim 16, for diagnosing or monitoring an ASCT1-associated disease in the subject.

29. A method of treating a subject in need thereof, comprising administering to said subject the polypeptide or functional variant thereof according to claim 16, a nucleic acid encoding said polypeptide or functional variant thereof, an expression vector comprising said nucleic acid, a cell comprising said nucleic acid or said expression vector, or a pharmaceutical composition comprising said nucleic acid, said expression vector or said cell and at least one pharmaceutically acceptable excipient.

30. A method of treating an ASCT1-associated disease in a subject in need thereof, by specifically targeting ASCT1 and not ASCT2, said method comprising administering to said subject the polypeptide or functional variant thereof according to claim 16, a nucleic acid encoding said polypeptide or functional variant thereof, an expression vector comprising said nucleic acid, a cell comprising said nucleic acid or said expression vector, or a pharmaceutical composition comprising said nucleic acid, said expression vector or said cell and at least one pharmaceutically acceptable excipient.

31. The method of specifically detecting or/and measuring the level of ASCT1 in vivo in a subject according to claim 27, for diagnosing or monitoring an ASCT1-associated disease in a subject.


SEQUENCE LISTING (60 sequences)

SEQ ID NO: 1 - protein, 59 aa, synthetic construct
  SITE 1: note = This residue is, or is not, linked N-terminally to a peptide having an undefined sequence of undefined length
  SITE 59: note = This residue is, or is not, linked C-terminally to a peptide having an undefined sequence of undefined length
YPLQQLWGLPCDCSGGYVSSIPTYYTYSLDCGGSTAYLTYGSGTGSWSWGGGFKQQWEC

SEQ ID NO: 2 - protein, 59 aa, synthetic construct; identical to SEQ ID NO: 1

SEQ ID NO: 3 - protein, 59 aa, synthetic construct
RERLEEIIRHSYPSVQYHPLALPRSRGVDLDPQTSDILEATHQVLNATNPKLAENCWLC

SEQ ID NO: 4 - protein, 60 aa, synthetic construct
YPLQQLWGLPCDCSGGYVSSIPTYYTYSLDCGGSTAYLTYGSGTGSWSWGGGFKQQWECV

SEQ ID NO: 5 - protein, 61 aa, synthetic construct
YPLQQLWGLPCDCSGGYVSSIPTYYTYSLDCGGSTAYLTYGSGTGSWSWGGGFKQQWECVF

SEQ ID NO: 6 - protein, 62 aa, synthetic construct
YPLQQLWGLPCDCSGGYVSSIPTYYTYSLDCGGSTAYLTYGSGTGSWSWGGGFKQQWECVFK

SEQ ID NO: 7 - protein, 63 aa, synthetic construct
YPLQQLWGLPCDCSGGYVSSIPTYYTYSLDCGGSTAYLTYGSGTGSWSWGGGFKQQWECVFKP

SEQ ID NO: 8 - protein, 104 aa, synthetic construct
VFKPKIIPSVQGQPGPCPSECLQIATQMHSTCYEKTQECTLLGKTYFTAILQKTKLGSYEDGPNKLIQASCTGTVGKPVCWDPVAPVYVSDGGGPTDMIREESV

SEQ ID NO: 9 - protein, 103 aa, synthetic construct
FKPKIIPSVQGQPGPCPSECLQIATQMHSTCYEKTQECTLLGKTYFTAILQKTKLGSYEDGPNKLIQASCTGTVGKPVCWDPVAPVYVSDGGGPTDMIREESV

SEQ ID NO: 10 - protein, 102 aa, synthetic construct
KPKIIPSVQGQPGPCPSECLQIATQMHSTCYEKTQECTLLGKTYFTAILQKTKLGSYEDGPNKLIQASCTGTVGKPVCWDPVAPVYVSDGGGPTDMIREESV

SEQ ID NO: 11 - protein, 101 aa, synthetic construct
PKIIPSVQGQPGPCPSECLQIATQMHSTCYEKTQECTLLGKTYFTAILQKTKLGSYEDGPNKLIQASCTGTVGKPVCWDPVAPVYVSDGGGPTDMIREESV

SEQ ID NO: 12 - protein, 100 aa, synthetic construct
KIIPSVQGQPGPCPSECLQIATQMHSTCYEKTQECTLLGKTYFTAILQKTKLGSYEDGPNKLIQASCTGTVGKPVCWDPVAPVYVSDGGGPTDMIREESV

SEQ ID NO: 13 - protein, 258 aa, synthetic construct
MDCLTNLRSAEGKVDQASKILILLVAWWGFGTTAEGYPLQQLWGLPCDCSGGYVSSIPTYYTYSLDCGGSTAYLTYGSGTGSWSWGGGFKQQWECVFKPKIIPSVQGQPGPCPSECLQIATQMHSTCYEKTQECTLLGKTYFTAILQKTKLGSYEDGPNKLIQASCTGTVGKPVCWDPVAPVYVSDGGGPTDMIREESVRERLEEIIRHSYPSVQYHPLALPRSRGVDLDPQTSDILEATHQVLNATNPKLAENCWLC

SEQ ID NO: 14 - DNA, 177 bp, synthetic construct
  source 1..177: mol_type = other DNA
taccccttgcagcaactttggggactgccttgtgactgttccgggggatatgtctcctccatacctacctattacacctactccctcgattgtggtggctccaccgcctacctgacttacggatctggtacagggagttggagctggggagggggatttaaacaacagtgggaatgt

SEQ ID NO: 15 - DNA, 180 bp, synthetic construct
  source 1..180: mol_type = other DNA
taccccttgcagcaactttggggactgccttgtgactgttccgggggatatgtctcctccatacctacctattacacctactccctcgattgtggtggctccaccgcctacctgacttacggatctggtacagggagttggagctggggagggggatttaaacaacagtgggaatgtgtg

SEQ ID NO: 16 - DNA, 183 bp, synthetic construct
  source 1..183: mol_type = other DNA
taccccttgcagcaactttggggactgccttgtgactgttccgggggatatgtctcctccatacctacctattacacctactccctcgattgtggtggctccaccgcctacctgacttacggatctggtacagggagttggagctggggagggggatttaaacaacagtgggaatgtgtgttt

SEQ ID NO: 17 - DNA, 186 bp, synthetic construct
  source 1..186: mol_type = other DNA
taccccttgcagcaactttggggactgccttgtgactgttccgggggatatgtctcctccatacctacctattacacctactccctcgattgtggtggctccaccgcctacctgacttacggatctggtacagggagttggagctggggagggggatttaaacaacagtgggaatgtgtgtttaaa

SEQ ID NO: 18 - DNA, 189 bp, synthetic construct
  source 1..189: mol_type = other DNA
taccccttgcagcaactttggggactgccttgtgactgttccgggggatatgtctcctccatacctacctattacacctactccctcgattgtggtggctccaccgcctacctgacttacggatctggtacagggagttggagctggggagggggatttaaacaacagtgggaatgtgtgtttaaacct

SEQ ID NO: 19 - DNA, 666 bp, synthetic construct
  source 1..666: mol_type = other DNA
taccccttgcagcaactttggggactgccttgtgactgttccgggggatatgtctcctccatacctacctattacacctactccctcgattgtggtggctccaccgcctacctgacttacggatctggtacagggagttggagctggggagggggatttaaacaacagtgggaatgtgtgtttaaacctaagatcataccctctgtgcaggggcagccagggccctgcccatctgaatgcctccagatagctactcaaatgcattccacttgttatgaaaagactcaggaatgcaccctcctgggaaaaacctattttactgccatcctacagaaaacaaagctaggctcgtatgaagacgggcctaataaactgatccaggcctcttgcacgggaaccgtagggaaaccagtatgttgggaccctgtagcccctgtgtatgtctctgatggcggcggtcccactgacatgattcgggaagagtctgtgcgtgaaagactagaggaaatcatcaggcacagctacccctccgtacagtatcaccctttagccctgccccgatcaagaggagtagatctggatccccagacgtctgacatactggaagctactcaccaggtccttaatgccactaatcccaagctagcagagaactgctggctttgt

SEQ ID NO: 20 - protein, 532 aa, Homo sapiens
MEKSNETNGYLDSAQAGPAAGPGAPGTAAGRARRCAGFLRRQALVLLTVSGVLAGAGLGAALRGLSLSRTQVTYLAFPGEMLLRMLRMIILPLVVCSLVSGAASLDASCLGRLGGIAVAYFGLTTLSASALAVALAFIIKPGSGAQTLQSSDLGLEDSGPPPVPKETVDSFLDLARNLFPSNLVVAAFRTYATDYKVVTQNSSSGNVTHEKIPIGTEIEGMNILGLVLFALVLGVALKKLGSEGEDLIRFFNSLNEATMVLVSWIMWYVPVGIMFLVGSKIVEMKDIIVLVTSLGKYIFASILGHVIHGGIVLPLIYFVFTRKNPFRFLLGLLAPFATAFATCSSSATLPSMMKCIEENNGVDKRISRFILPIGATVNMDGAAIFQCVAAVFIAQLNNVELNAGQIFTILVTATASSVGAAGVPAGGVLTIAIILEAIGLPTHDLPLILAVDWIVDRTTTVVNVEGDALGAGILHHLNQKATKKGEQELAEVKVEAIPNCKSEEETSPLVTHQNPAGPVASAPELESKESVL

SEQ ID NO: 21 - RNA, 2102 nt, Homo sapiens
  source 1..2102: mol_type = mRNA
cccgcactctgcgcctctcctcgcctttctcgcacctgctcctgcgccaggcccggagacccccggggcggcttcccagaacctgcggagcacaactggccgaccgacccattcattgggaacccgtcttttgccagagcccacgtcccctgccacctctagctcggagcggcgtgtagcgccatggagaagagcaacgagaccaacggctaccttgacagcgctcaggcggggcctgcggccgggcccggagctccggggaccgcggcgggacgcgcacggcgttgcgcgcgcttcctgcggcgccaagcgctggtgctgctcaccgtgtccggggtgctggcgggcgcgggcctgggcgcggcgttgcgcgggctcagcctgagccgcacgcaggtcacctacctggccttccccggcgagatgctgctccgcatgctgcgcatgatcatcctgccgctggtggtctgcagcctggtgtcgggcgccgcctcgctcgatgccagctgcctcgggcgtctgggcggcatccgtgtcgcctactttggcctcaccacactgagtgcctcggcgctcgccgtggccttggcgttcatcatcaagccaggatccggtgcgcagacccttcagtccagcgacctggggctggaggactcggggcctcctcctgtccccaaagagacggtggactctttcctcgacctggccagaaacctgtttccctccaatcttgtggttgcagctttccgtacgtatgcaaccgattataaagtcgtgacccagaacagcagctctggaaatgtaacccatgaaaagatccccataggcactgagatagaagggatgaacattttaggattggtcctgtttgctctggtgttaggagtggccttaaagaaactaggctccgaaggagaagacctcatccgtttcttcaattccctcaacgaggcgacgatggtgctggtgtcctggattatgtggtacgtacctgtgggcatcatgttccttgttggaagcaagatcgtggaaatgaaagacatcatcgtgctggtgaccagcctggggaaatacatcttcgcatctatattgggccatgttattcatggaggaattgttctgccacttatttattttgttttcacacgaaaaaacccattcagattcctcctgggcctcctcgccccatttgcgacagcatttgctacctgctccagctcagcgacccttccctctatgatgaagtgcattgaagagaacaatggtgtggacaagaggatcagcaggtttattctccccatcggggccaccgtgaacatggacggagcagccatcttccagtgtgtggccgcggtgttcattgcgcaactcaacaacatagagctcaacgcaggacagattttcaccattctagtgactgccacagcgtccagtgttggagcagcaggcgtgccagctggaggggtcctcaccattgccattatcctggaggccattgggctgcctactcatgacctgcctctgatcctggctgtggactggattgtggaccggaccaccacggtggtgaatgtggaaggggatgccctgggtgcaggcattctccaccacctgaatcagaaggcaacaaagaaaggcgagcaggaacttgctgaggtgaaagtggaagccatccccaactgcaagtctgaggaggagacatcgcccctggtgacacaccagaaccccgctggccccgtggccagtgccccagaactggaatccaaggagtcggttctgtgatggggctgggctttgggcttgcctgccagcagtgatgtcccaccctgttcacccagccgccagtcatggacacagggcactgccttgccaacttttaccctcccaagcaatgctttggcccagtcgctggcctgaggcttacctctcggcactggcattgggctccccagccggaactggttaccaaggacaaggacactctgacattcggcttgatccatgtccaggtgcaactgtgtgtacaccagggatctgtttggaaacaaccccttgagctgccaggctcaagaaatcatggactcacagggtcctgtgtggtt

SEQ ID NO: 22 - protein, 541 aa, Homo sapiens
MVADPPRDSKGLAAAEPTANGGLALASIEDQGAAAGGYCGSRDQVRRCLRANLLVLLTVVAVVAGVALGLGVSGAGGALALGPERLSAFVFPGELLLRLLRMIILPLVVCSLIGGAASLDPGALGRLGAWALLFFLVTTLLASALGVGLALALQPGAASAAINASVGAAGSAENAPSKEVLDSFLDLARNIFPSNLVSAAFRSYSTTYEERNITGTRVKVPVGQEVEGMNILGLVVFAIVFGVALRKLGPEGELLIRFFNSFNEATMVLVSWIMWYAPVGIMFLVAGKIVEMEDVGLLFARLGKYILCCLLGHAIHGLLVLPLIYFLFTRKNPYRFLWGIVTPLATAFGTSSSSATLPLMMKCVEENNGVAKHISRFILPIGATVNMDGAALFQCVAAVFIAQLSQQSLDFVKIITILVTATASSVGAAGIPAGGVLTLAIILEAVNLPVDHISLILAVDWLVDRSCTVLNVEGDALGAGLLQNYVDRTESRSTEPELIQVKSELPLDPLPVPTEEGNPLLKHYRGPAGDATVASEKESVM

SEQ ID NO: 23 - RNA, 1626 nt, Homo sapiens
  source 1..1626: mol_type = mRNA
atggtggccgatcctcctcgagactccaaggggctcgcagcggcggagcccaccgccaacgggggcctggcgctggcctccatcgaggaccaaggcgcggcagcaggcggctactgcggttcccgggaccaggtgcgccgctgccttcgagccaacctgcttgtgctgctgacagtggtggccgtggtggccggcgtggcgctgggactgggggtgtcgggggccgggggtgcgctggcgttgggcccggagcgcttgagcgccttcgtcttcccgggcgagctgctgctgcgtctgctgcggatgatcatcttgccgctggtggtgtgcagcttgatcggcggcgccgccagcctggaccccggcgcgctcggccgtctgggcgcctgggcgctgctctttttcctggtcaccacgctgctggcgtcggcgctcggagtgggcttggcgctggctctgcagccgggcgccgcctccgccgccatcaacgcctccgtgggagccgcgggcagtgccgaaaatgcccccagcaaggaggtgctcgattcgttcctggatcttgcgagaaatatcttcccttccaacctggtgtcagcagcctttcgctcatactctaccacctatgaagagaggaatatcaccggaaccagggtgaaggtgcccgtggggcaggaggtggaggggatgaacatcctgggcttggtagtgtttgccatcgtctttggtgtggcgctgcggaagctggggcctgaaggggagctgcttatccgcttcttcaactccttcaatgaggccaccatggttctggtctcctggatcatgtggtatgcccctgtgggcatcatgttcctggtggctggcaagatcgtggagatggaggatgtgggtttactctttgcccgccttggcaagtacattctgtgctgcctgctgggtcacgccatccatgggctcctggtactgcccctcatctacttcctcttcacccgcaaaaacccctaccgcttcctgtggggcatcgtgacgccgctggccactgcctttgggacctcttccagttccgccacgctgccgctgatgatgaagtgcgtggaggagaataatggcgtggccaagcacatcagccgtttcatcctgcccatcggcgccaccgtcaacatggacggtgccgcgctcttccagtgcgtggccgcagtgttcattgcacagctcagccagcagtccttggacttcgtaaagatcatcaccatcctggtcacggccacagcgtccagcgtgggggcagcgggcatccctgctggaggtgtcctcactctggccatcatcctcgaagcagtcaacctcccggtcgaccatatctccttgatcctggctgtggactggctagtcgaccggtcctgtaccgtcctcaatgtagaaggtgacgctctgggggcaggactcctccaaaattacgtggaccgtacggagtcgagaagcacagagcctgagttgatacaagtgaagagtgagctgcccctggatccgctgccagtccccactgaggaaggaaaccccctcctcaaacactatcgggggcccgcaggggatgccacggtcgcctctgagaaggaatcagtcatgtaa

SEQ ID NO: 24 - protein, 228 aa, synthetic construct
APSTCSKPTCPPPELLGGPSVFIFPPKPKDTLMISRTPEVTCVVVDVSQDDPEVQFTWYINNEQVRTARPPLREQQFDCTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 25 - DNA, 687 bp, synthetic construct
  source 1..687: mol_type = other DNA
gcaccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcacgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacaacaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 26 - protein, 229 aa, synthetic construct
VDVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 27 - DNA, 690 bp, synthetic construct
  source 1..690: mol_type = other DNA
gtcgacgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 28 - protein, 20 aa, synthetic construct
MYRMQLLSCIALSLALVTNS

SEQ ID NO: 29 - protein, 18 aa, synthetic construct
MKWVTFISLLFLFSSAYS

SEQ ID NO: 30 - protein, 18 aa, synthetic construct
MAFLWLLSCWALLGTTFG

SEQ ID NO: 31 - protein, 15 aa, synthetic construct
MNLLLILTFVAAAVA

SEQ ID NO: 32 - protein, 17 aa, synthetic construct
MGVKVLFALICIAVAEA

SEQ ID NO: 33 - protein, 21 aa, synthetic construct
MKFSWVMFFLMAVVTGVNSEF

SEQ ID NO: 34 - protein, 431 aa, synthetic construct
MDCLTNLRSAEGKVDQASKILILLVAWWGFGTTAEGYPLQQLWGLPCDCSGGYVSSIPTYYTYSLDCGGSTAYLTYGSGTGSWSWGGGFKQQWECVFKPKIIPSVQGQPGPCPSECLQIATQMHSTCYEKTQECTLLGKTYFTAILQKTKLGSYEDGPNKLIQASCTGTVGKPVCWDPVAPVYVSDGGGPTDMIREESVRRSVDVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 35 - protein, 426 aa, synthetic construct
MDCLTNLRSAEGKVDQASKILILLVAWWGFGTTAEGYPLQQLWGLPCDCSGGYVSSIPTYYTYSLDCGGSTAYLTYGSGTGSWSWGGGFKQQWECVFKPKIIPSVQGQPGPCPSECLQIATQMHSTCYEKTQECTLLGKTYFTAILQKTKLGSYEDGPNKLIQASCTGTVGKPVCWDPVAPVYVSDGGGPTDMIRRSVDVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 36 - protein, 330 aa, synthetic construct
MDCLTNLRSAEGKVDQASKILILLVAWWGFGTTAEGYPLQQLWGLPCDCSGGYVSSIPTYYTYSLDCGGSTAYLTYGSGTGSWSWGGGFKQQWECVFKPRSVDVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 37 - protein, 320 aa, synthetic construct
MDCLTNLRSAEGKVDQASKILILLVAWWGFGTTAEGYPLQQLWGLPCDCSGGYVSSIPTYYTYSLDCGGSTAYLTYGSGTGSWSWGGGFRSVDVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 38 - DNA, 1296 bp, synthetic construct
  source 1..1296: mol_type = other DNA
atggactgtctcaccaacctccgatccgctgagggtaaagttgaccaggcgagcaaaatcctaattctccttgtggcttggtgggggtttgggaccactgccgaaggttaccccttgcagcaactttggggactgccttgtgactgttccgggggatatgtctcctccatacctacctattacacctactccctcgattgtggtggctccaccgcctacctgacttacggatctggtacagggagttggagctggggagggggatttaaacaacagtgggaatgtgtgtttaaacctaagatcataccctctgtgcaggggcagccagggccctgcccatctgaatgcctccagatagctactcaaatgcattccacttgttatgaaaagactcaggaatgcaccctcctgggaaaaacctattttactgccatcctacagaaaacaaagctaggctcgtatgaagacgggcctaataaactgatccaggcctcttgcacgggaaccgtagggaaaccagtatgttgggaccctgtagcccctgtgtatgtctctgatggcggcggtcccactgacatgattcgggaagagtctgtgcgtagatctgtcgacgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 39 - DNA, 1281 bp, synthetic construct
  source 1..1281: mol_type = other DNA
atggactgtctcaccaacctccgatccgctgagggtaaagttgaccaggcgagcaaaatcctaattctccttgtggcttggtgggggtttgggaccactgccgaaggttaccccttgcagcaactttggggactgccttgtgactgttccgggggatatgtctcctccatacctacctattacacctactccctcgattgtggtggctccaccgcctacctgacttacggatctggtacagggagttggagctggggagggggatttaaacaacagtgggaatgtgtgtttaaacctaagatcataccctctgtgcaggggcagccagggccctgcccatctgaatgcctccagatagctactcaaatgcattccacttgttatgaaaagactcaggaatgcaccctcctgggaaaaacctattttactgccatcctacagaaaacaaagctaggctcgtatgaagacgggcctaataaactgatccaggcctcttgcacgggaaccgtagggaaaccagtatgttgggaccctgtagcccctgtgtatgtctctgatggcggcggtcccactgacatgattcggagatctgtcgacgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 40 - DNA, 993 bp, synthetic construct
  source 1..993: mol_type = other DNA
atggactgtctcaccaacctccgatccgctgagggtaaagttgaccaggcgagcaaaatcctaattctccttgtggcttggtgggggtttgggaccactgccgaaggttaccccttgcagcaactttggggactgccttgtgactgttccgggggatatgtctcctccatacctacctattacacctactccctcgattgtggtggctccaccgcctacctgacttacggatctggtacagggagttggagctggggagggggatttaaacaacagtgggaatgtgtgtttaaacctagatctgtcgacgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 41 - DNA, 963 bp, synthetic construct
  source 1..963: mol_type = other DNA
atggactgtctcaccaacctccgatccgctgagggtaaagttgaccaggcgagcaaaatcctaattctccttgtggcttggtgggggtttgggaccactgccgaaggttaccccttgcagcaactttggggactgccttgtgactgttccgggggatatgtctcctccatacctacctattacacctactccctcgattgtggtggctccaccgcctacctgacttacggatctggtacagggagttggagctggggagggggatttagatctgtcgacgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 42 - DNA, 36 bp, synthetic construct
  source 1..36: mol_type = other DNA
ggcggccgcaccaccatggactgtctcaccaacctc

SEQ ID NO: 43 - DNA, 33 bp, synthetic construct
  source 1..33: mol_type = other DNA
cccagatctaaatccccctccccagctccaact

SEQ ID NO: 44 - DNA, 36 bp, synthetic construct
  source 1..36: mol_type = other DNA
cccagatctaggtttaaacacacattcccactgttg

SEQ ID NO: 45 - DNA, 30 bp, synthetic construct
  source 1..30: mol_type = other DNA
cccagatctccgaatcatgtcagtgggacc

SEQ ID NO: 46 - DNA, 33 bp, synthetic construct
  source 1..33: mol_type = other DNA
cccagatctacgcacagactcttcccgaatcat

SEQ ID NO: 47 - DNA, 33 bp, synthetic construct
  source 1..33: mol_type = other DNA
cccagatctacaaagccagcagttctctgctag

SEQ ID NO: 48 - protein, 36 aa, synthetic construct
MDCLTNLRSAEGKVDQASKILILLVAWWGFGTTAEG

SEQ ID NO: 49 - protein, 325 aa, synthetic construct
MDCLTNLRSAEGKVDQASKILILLVAWWGFGTTAEGYPLQQLWGLPCDCSGGYVSSIPTYYTYSLDCGGSTAYLTYGSGTGSWSWGGGFKQQWERSVDVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 50 - protein, 328 aa, synthetic construct
MDCLTNLRSAEGKVDQASKILILLVAWWGFGTTAEGYPLQQLWGLPCDCSGGYVSSIPTYYTYSLDCGGSTAYLTYGSGTGSWSWGGGFKQQWECVFRSVDVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 51 - protein, 329 aa, synthetic construct
MDCLTNLRSAEGKVDQASKILILLVAWWGFGTTAEGYPLQQLWGLPCDCSGGYVSSIPTYYTYSLDCGGSTAYLTYGSGTGSWSWGGGFKQQWECVFKRSVDVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 52 - protein, 441 aa, synthetic construct
MDCLTNLRSAEGKVDQASKILILLVAWWGFGTTAEGYPLQQLWGLPCDCSGGYVSSIPTYYTYSLDCGGSTAYLTYGSGTGSWSWGGGFKQQWECVFKPKIIPSVQGQPGPCPSECLQIATQMHSTCYEKTQECTLLGKTYFTAILQKTKLGSYEDGPNKLIQASCTGTVGKPVCWDPVAPVYVSDGGGPTDMIREESVRERLEEIIRHSRSVDVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 53 - protein, 451 aa, synthetic construct
MDCLTNLRSAEGKVDQASKILILLVAWWGFGTTAEGYPLQQLWGLPCDCSGGYVSSIPTYYTYSLDCGGSTAYLTYGSGTGSWSWGGGFKQQWECVFKPKIIPSVQGQPGPCPSECLQIATQMHSTCYEKTQECTLLGKTYFTAILQKTKLGSYEDGPNKLIQASCTGTVGKPVCWDPVAPVYVSDGGGPTDMIREESVRERLEEIIRHSYPSVQYHPLARSVDVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 54 - protein, 489 aa, synthetic construct
MDCLTNLRSAEGKVDQASKILILLVAWWGFGTTAEGYPLQQLWGLPCDCSGGYVSSIPTYYTYSLDCGGSTAYLTYGSGTGSWSWGGGFKQQWECVFKPKIIPSVQGQPGPCPSECLQIATQMHSTCYEKTQECTLLGKTYFTAILQKTKLGSYEDGPNKLIQASCTGTVGKPVCWDPVAPVYVSDGGGPTDMIREESVRERLEEIIRHSYPSVQYHPLALPRSRGVDLDPQTSDILEATHQVLNATNPKLAENCWLCRSVDVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 55 - DNA, 978 bp, synthetic construct
  source 1..978: mol_type = other DNA
atggactgtctcaccaacctccgatccgctgagggtaaagttgaccaggcgagcaaaatcctaattctccttgtggcttggtgggggtttgggaccactgccgaaggttaccccttgcagcaactttggggactgccttgtgactgttccgggggatatgtctcctccatacctacctattacacctactccctcgattgtggtggctccaccgcctacctgacttacggatctggtacagggagttggagctggggagggggatttaaacaacagtgggaaagatctgtcgacgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 56 - DNA, 987 bp, synthetic construct
  source 1..987: mol_type = other DNA
atggactgtctcaccaacctccgatccgctgagggtaaagttgaccaggcgagcaaaatcctaattctccttgtggcttggtgggggtttgggaccactgccgaaggttaccccttgcagcaactttggggactgccttgtgactgttccgggggatatgtctcctccatacctacctattacacctactccctcgattgtggtggctccaccgcctacctgacttacggatctggtacagggagttggagctggggagggggatttaaacaacagtgggaatgtgtgtttagatctgtcgacgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 57 - DNA, 990 bp, synthetic construct
  source 1..990: mol_type = other DNA
atggactgtctcaccaacctccgatccgctgagggtaaagttgaccaggcgagcaaaatcctaattctccttgtggcttggtgggggtttgggaccactgccgaaggttaccccttgcagcaactttggggactgccttgtgactgttccgggggatatgtctcctccatacctacctattacacctactccctcgattgtggtggctccaccgcctacctgacttacggatctggtacagggagttggagctggggagggggatttaaacaacagtgggaatgtgtgtttaaaagatctgtcgacgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 58 - DNA, 1326 bp, synthetic construct
  source 1..1326: mol_type = other DNA
atggactgtctcaccaacctccgatccgctgagggtaaagttgaccaggcgagcaaaatcctaattctccttgtggcttggtgggggtttgggaccactgccgaaggttaccccttgcagcaactttggggactgccttgtgactgttccgggggatatgtctcctccatacctacctattacacctactccctcgattgtggtggctccaccgcctacctgacttacggatctggtacagggagttggagctggggagggggatttaaacaacagtgggaatgtgtgtttaaacctaagatcataccctctgtgcaggggcagccagggccctgcccatctgaatgcctccagatagctactcaaatgcattccacttgttatgaaaagactcaggaatgcaccctcctgggaaaaacctattttactgccatcctacagaaaacaaagctaggctcgtatgaagacgggcctaataaactgatccaggcctcttgcacgggaaccgtagggaaaccagtatgttgggaccctgtagcccctgtgtatgtctctgatggcggcggtcccactgacatgattcgggaagagtctgtgcgtgaaagactagaggaaatcatcaggcacagcagatctgtcgacgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 59 - DNA, 1356 bp, synthetic construct
  source 1..1356: mol_type = other DNA
atggactgtctcaccaacctccgatccgctgagggtaaagttgaccaggcgagcaaaatcctaattctccttgtggcttggtgggggtttgggaccactgccgaaggttaccccttgcagcaactttggggactgccttgtgactgttccgggggatatgtctcctccatacctacctattacacctactccctcgattgtggtggctccaccgcctacctgacttacggatctggtacagggagttggagctggggagggggatttaaacaacagtgggaatgtgtgtttaaacctaagatcataccctctgtgcaggggcagccagggccctgcccatctgaatgcctccagatagctactcaaatgcattccacttgttatgaaaagactcaggaatgcaccctcctgggaaaaacctattttactgccatcctacagaaaacaaagctaggctcgtatgaagacgggcctaataaactgatccaggcctcttgcacgggaaccgtagggaaaccagtatgttgggaccctgtagcccctgtgtatgtctctgatggcggcggtcccactgacatgattcgggaagagtctgtgcgtgaaagactagaggaaatcatcaggcacagctacccctccgtacagtatcaccctttagccagatctgtcgacgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 60 - DNA, 1470 bp, synthetic construct
  source 1..1470: mol_type = other DNA
atggactgtctcaccaacctccgatccgctgagggtaaagttgaccaggcgagcaaaatcctaattctccttgtggcttggtgggggtttgggaccactgccgaaggttaccccttgcagcaactttggggactgccttgtgactgttccgggggatatgtctcctccatacctacctattacacctactccctcgattgtggtggctccaccgcctacctgacttacggatctggtacagggagttggagctggggagggggatttaaacaacagtgggaatgtgtgtttaaacctaagatcataccctctgtgcaggggcagccagggccctgcccatctgaatgcctccagatagctactcaaatgcattccacttgttatgaaaagactcaggaatgcaccctcctgggaaaaacctattttactgccatcctacagaaaacaaagctaggctcgtatgaagacgggcctaataaactgatccaggcctcttgcacgggaaccgtagggaaaccagtatgttgggaccctgtagcccctgtgtatgtctctgatggcggcggtcccactgacatgattcgggaagagtctgtgcgtgaaagactagaggaaatcatcaggcacagctacccctccgtacagtatcaccctttagccctgccccgatcaagaggagtagatctggatccccagacgtctgacatactggaagctactcaccaggtccttaatgccactaatcccaagctagcagagaactgctggctttgtagatctgtcgacgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga